SEQUENCE LISTING (77 sequences)

SEQ ID NO: 1 - protein, 3396 aa, Homo sapiens
MFINIKSILWMCSTLIVTHALHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEATTIDLSILAETASPSLSKEPQMVSDRTTPIIPLVDELPVIPTEFPPVGNIVSFEQKATVQPQAITDSLATKLPTPTGSTKKPWDMDDYSPSASGPLGKLDISEIKEEVLQSTTGVSHYATDSWDGVVEDKQTQESVTQIEQIEVGPLVTSMEILKHIPSKEFPVTETPLVTARMILESKTEKKMVSTVSELVTTGHYGFTLGEEDDEDRTLTVGSDESTLIFDQIPEVITVSKTSEDTIHTHLEDLESVSASTTVSPLIMPDNNGSSMDDWEERQTSGRITEEFLGKYLSTTPFPSQHRTEIELFPYSGDKILVEGISTVIYPSLQTEMTHRRERTETLIPEMRTDTYTDEIQEEITKSPFMGKTEEEVFSGMKLSTSLSEPIHVTESSVEMTKSFDFPTLITKLSAEPTEVRDMEEDFTATPGTTKYDENITTVLLAHGTLSVEAATVSKWSWDEDNTTSKPLESTEPSASSKLPPALLTTVGMNGKDKDIPSFTEDGADEFTLIPDSTQKQLEEVTDEDIAAHGKFTIRFQPTTSTGIAEKSTLRDSTTEEKVPPITSTEGQVYATMEGSALGEVEDVDLSKPVSTVPQFAHTSEVEGLAFVSYSSTQEPTTYVDSSHTIPLSVIPKTDWGVLVPSVPSEDEVLGEPSQDILVIDQTRLEATISPETMRTTKITEGTTQEEFPWKEQTAEKPVPALSSTAWTPKEAVTPLDEQEGDGSAYTVSEDELLTGSERVPVLETTPVGKIDHSVSYPPGAVTEHKVKTDEVVTLTPRIGPKVSLSPGPEQKYETEGSSTTGFTSSLSPFSTHITQLMEETTTEKTSLEDIDLGSGLFEKPKATELIEFSTIKVTVPSDITTAFSSVDRLHTTSAFKPSSAITKKPPLIDREPGEETTSDMVIIGESTSHVPPTTLEDIVAKETETDIDREYFTTSSPPATQPTRPPTVEDKEAFGPQALSTPQPPASTKFHPDINVYIIEVRENKTGRMSDLSVIGHPIDSESKEDEPCSEETDPVHDLMAEILPEFPDIIEIDLYHSEENEEEEEECANATDVTTTPSVQYINGKHLVTTVPKDPEAAEARRGQFESVAPSQNFSDSSESDTHPFVIAKTELSTAVQPNESTETTESLEVTWKPETYPETSEHFSGGEPDVFPTVPFHEEFESGTAKKGAESVTERDTEVGHQAHEHTEPVSLFPEESSGEIAIDQESQKIAFARATEVTFGEEVEKSTSVTYTPTIVPSSASAYVSEEEAVTLIGNPWPDDLLSTKESWVEATPRQVVELSGSSSIPITEGSGEAEEDEDTMFTMVTDLSQRNTTDTLITLDTSRIITESFFEVPATTIYPVSEQPSAKVVPTKFVSETDTSEWISSTTVEEKKRKEEEGTTGTASTFEVYSSTQRSDQLILPFELESPNVATSSDSGTRKSFMSLTTPTQSEREMTDSTPVFTETNTLENLGAQTTEHSSIHQPGVQEGLTTLPRSPASVFMEQGSGEAAADPETTTVSSFSLNVEYAIQAEKEVAGTLSPHVETTFSTEPTGLVLSTVMDRVVAENITQTSREIVISERLGEPNYGAEIRGFSTGFPLEEDFSGDFREYSTVSHPIAKEETVMMEGSGDAAFRDTQTSPSTVPTSVHISHISDSEGPSSTMVSTSAFPWEEFTSSAEGSGEQLVTVSSSVVPVLPSAVQKFSGTASSIIDEGLGEVGTVNEIDRRSTILPTAEVEGTKAPVEKEEVKVSGTVSTNFPQTIEPAKLWSRQEVNPVRQEIESETTSEEQIQEEKSFESPQNSPATEQTIFDSQTFTETELKTTDYSVLTTKKTYSDDKEMKEEDTSLVNMSTPDPDANGLESYTTLPEATEKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTDFLARAYGFEMAKEFVTSAPKPSDLYYEPSGEGSGEVDIVDSFHTSATTQATRQESSTTFVSDGSLEKHPEVPSAKAVTADGFPTVSVMLPLHSEQNKSSPDPTSTLSNTVSYERSTDGSFQDRFREFEDSTLKPNRKKPTENIIIDLDKEDKDLILTITESTILEILPELTSDKNTIIDIDHTKPVYEDILGMQTDIDTEVPSEPHDSNDESNDDSTQVQEIYEAAVNLSLTEETFEGSADVLASYTQATHDESMTYEDRSQLDHMGFHFTTGIPAPSTETELDVLLPTATSLPIPRKSATVIPEIEGIKAEAKALDDMFESSTLSDGQAIADQSEIIPTLGQFERTQEEYEDKKHAGPSFQPEFSSGAEEALVDHTPYLSIATTHLMDQSVTEVPDVMEGSNPPYYTDTTLAVSTFAKLSSQTPSSPLTIYSGSEASGHTEIPQPSALPGIDVGSSVMSPQDSFKEIHVNIEATFKPSSEEYLHITEPPSLSPDTKLEPSEDDGKPELLEEMEASPTELIAVEGTEILQDFQNKTDGQVSGEAIKMFPTIKTPEAGTVITTADEIELEGATQWPHSTSASATYGVEAGVVPWLSPQTSERPTLSSSPEINPETQAALIRGQDSTIAASEQQVAARILDSNDQATVNPVEFNTEVATPPFSLLETSNETDFLIGINEESVEGTAIYLPGPDRCKMNPCLNGGTCYPTETSYVCTCVPGYSGDQCELDFDECHSNPCRNGATCVDGFNTFRCLCLPSYVGALCEQDTETCDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKGTVACGQPPVVENAKTFGKMKPRYEINSLIRYHCKDGFIQRHLPTIRCLGNGRWAIPKITCMNPSAYQRTYSMKYFKNSSSAKDNSINTSKHDHRWSRRWQESRR

SEQ ID NO: 2 - protein, 3396 aa, Homo sapiens; identical to SEQ ID NO: 1

SEQ ID NO: 3 - DNA, 12416 bp, Homo sapiens
cttcttctcgctgagtctcctcctcggctctgacggtacagtgatataatgatgatgggtgtcacaacccgcatttgaacttgcaggcgagctgccccgagcctttctggggaagaactccaggcgtgcggacgcaacagccgagaacattaggtgttgtggacaggagctgggaccaagatcttcggccagccccgcatcctcccgcatcttccagcaccgtcccgcaccctccgcatccttccccgggccaccacgcttcctatgtgacccgcctgggcaacgccgaacccagtcgcgcagcgctgcagtgaattttccccccaaactgcaataagccgccttccaaggccaagatgttcataaatataaagagcatcttatggatgtgttcaaccttaatagtaacccatgcgctacataaagtcaaagtgggaaaaagcccaccggtgaggggctccctctctggaaaagtcagcctaccttgtcatttttcaacgatgcctactttgccacccagttacaacaccagtgaatttctccgcatcaaatggtctaagattgaagtggacaaaaatggaaaagatttgaaagagactactgtccttgtggcccaaaatggaaatatcaagattggtcaggactacaaagggagagtgtctgtgcccacacatcccgaggctgtgggcgatgcctccctcactgtggtcaagctgctggcaagtgatgcgggtctttaccgctgtgacgtcatgtacgggattgaagacacacaagacacggtgtcactgactgtggatggggttgtgtttcactacagggcggcaaccagcaggtacacactgaattttgaggctgctcagaaggcttgtttggacgttggggcagtcatagcaactccagagcagctctttgctgcctatgaagatggatttgagcagtgtgacgcaggctggctggctgatcagactgtcagatatcccatccgggctcccagagtaggctgttatggagataagatgggaaaggcaggagtcaggacttatggattccgttctccccaggaaacttacgatgtgtattgttatgtggatcatctggatggtgatgtgttccacctcactgtccccagtaaattcaccttcgaggaggctgcaaaagagtgtgaaaaccaggatgccaggctggcaacagtgggggaactccaggcggcatggaggaacggctttgaccagtgcgattacgggtggctgtcggatgccagcgtgcgccaccctgtgactgtggccagggcccagtgtggaggtggtctacttggggtgagaaccctgtatcgttttgagaaccagacaggcttccctccccctgatagcagatttgatgcctactgctttaaacctaaagaggctacaaccatcgatttgagtatcctcgcagaaactgcatcacccagtttatccaaagaaccacaaatggtttctgatagaactacaccaatcatccctttagttgatgaattacctgtcattccaacagagttccctcccgtgggaaatattgtcagttttgaacagaaagccacagtccaacctcaggctatcacagatagtttagccaccaaattacccacacctactggcagtaccaagaagccctgggatatggatgactactcaccttctgcttcaggacctcttggaaagctagacatatcagaaattaaggaagaagtgctccagagtacaactggcgtctctcattatgctacggattcatgggatggtgtcgtggaagataaacaaacacaagaatcggttacacagattgaacaaatagaagtgggtcctttggtaacatctatggaaatcttaaagcacattccttccaaggaattccctgtaactgaaacaccattggtaactgcaagaatgatcctggaatccaaaactgaaaagaaaatggtaagcactgtttctgaattggtaaccacaggtcactatggattcaccttgggagaagaggatgatgaagacagaacacttacagttggatctgatgagagcaccttgatctttgaccaaattcctgaagtcattacggtgtcaaagacttcagaagacaccatccacactcatttagaagacttggagtcagtctcagcatccacaactgtttcccctttaattatgcctgataataatggatcatccatggatgactgggaagagagacaaactagtggtaggataacggaagagtttcttggcaaatatctgtctactacaccttttccatcacagcatcgtacagaaatagaattgtttccttattctggtgataaaatattagtagagggaatttccacagttatttatccttctctacaaacagaaatgacacatagaagagaaagaacagaaacactaataccagagatgagaacagatacttatacagatgaaatacaagaagagatcactaaaagtccatttatgggaaaaacagaagaagaagtcttctctgggatgaaactctctacatctctctcagagccaattcatgttacagagtcttctgtggaaatgaccaagtcttttgatttcccaacattgataacaaagttaagtgcagagccaacagaagtaagagatatggaggaagactttacagcaactccaggtactacaaaatatgatgaaaatattacaacagtgcttttggcccatggtactttaagtgttgaagcagccactgtatcaaaatggtcatgggatgaagataatacaacatccaagcctttagagtctacagaaccttcagcctcttcaaaattgccccctgccttactcacaactgtggggatgaatggaaaggataaagacatcccaagtttcactgaagatggagcagatgaatttactcttattccagatagtactcaaaagcagttagaggaggttactgatgaagacatagcagcccatggaaaattcacaattagatttcagccaactacatcaactggtattgcagaaaagtcaactttgagagattctacaactgaagaaaaagttccacctatcacaagcactgaaggccaagtttatgcaaccatggaaggaagtgctttgggtgaagtagaagatgtggacctctctaagccagtatctactgttccccaatttgcacacacttcagaggtggaaggattagcatttgttagttatagtagcacccaagagcctactacttatgtagactcttcccataccattcctctttctgtaattcccaagacagactggggagtgttagtaccttctgttccatcagaagatgaagttctaggtgaaccctctcaagacatacttgtcattgatcagactcgccttgaagcgactatttctccagaaactatgagaacaacaaaaatcacagagggaacaactcaggaagaattcccttggaaagaacagactgcagagaaaccagttcctgctctcagttctacagcttggactcccaaggaggcagtaacaccactggatgaacaagagggcgatggatcagcatatacagtctctgaagatgaattgttgacaggttctgagagggtcccagttttagaaacaactccagttggaaaaattgatcacagtgtgtcttatccaccaggtgctgtaactgagcacaaagtgaaaacagatgaagtggtaacactaacaccacgcattgggccaaaagtatctttaagtccagggcctgaacaaaaatatgaaacagaaggtagtagtacaacaggatttacatcatctttgagtccttttagtacccacattacccagcttatggaagaaaccactactgagaaaacatccctagaggatattgatttaggctcaggattatttgaaaagcccaaagccacagaactcatagaattttcaacaatcaaagtcacagttccaagtgatattaccactgccttcagttcagtagacagacttcacacaacttcagcattcaagccatcttccgcgatcactaagaaaccacctctcatcgacagggaacctggtgaagaaacaaccagtgacatggtaatcattggagaatcaacatctcatgttcctcccactacccttgaagatattgtagccaaggaaacagaaaccgatattgatagagagtatttcacgacttcaagtcctcctgctacacagccaacaagaccacccactgtggaagacaaagaggcctttggacctcaggcgctttctacgccacagcccccagcaagcacaaaatttcaccctgacattaatgtttatattattgaggtcagagaaaataagacaggtcgaatgagtgatttgagtgtaattggtcatccaatagattcagaatctaaagaagatgaaccttgtagtgaagaaacagatccagtgcatgatctaatggctgaaattttacctgaattccctgacataattgaaatagacctataccacagtgaagaaaatgaagaagaagaagaagagtgtgcaaatgctactgatgtgacaaccaccccatctgtgcagtacataaatgggaagcatctcgttaccactgtgcccaaggacccagaagctgcagaagctaggcgtggccagtttgaaagtgttgcaccttctcagaatttctcggacagctctgaaagtgatactcatccatttgtaatagccaaaacggaattgtctactgctgtgcaacctaatgaatctacagaaacaactgagtctcttgaagttacatggaagcctgagacttaccctgaaacatcagaacatttttcaggtggtgagcctgatgttttccccacagtcccattccatgaggaatttgaaagtggaacagccaaaaaaggggcagaatcagtcacagagagagatactgaagttggtcatcaggcacatgaacatactgaacctgtatctctgtttcctgaagagtcttcaggagagattgccattgaccaagaatctcagaaaatagcctttgcaagggctacagaagtaacatttggtgaagaggtagaaaaaagtacttctgtcacatacactcccactatagttccaagttctgcatcagcatatgtttcagaggaagaagcagttaccctaataggaaatccttggccagatgacctgttgtctaccaaagaaagctgggtagaagcaactcctagacaagttgtagagctctcagggagttcttcgattccaattacagaaggctctggagaagcagaagaagatgaagatacaatgttcaccatggtaactgatttatcacagagaaatactactgatacactcattactttagacactagcaggataatcacagaaagcttttttgaggttcctgcaaccaccatttatccagtttctgaacaaccttctgcaaaagtggtgcctaccaagtttgtaagtgaaacagacacttctgagtggatttccagtaccactgttgaggaaaagaaaaggaaggaggaggagggaactacaggtacggcttctacatttgaggtatattcatctacacagagatcggatcaattaattttaccctttgaattagaaagtccaaatgtagctacatctagtgattcaggtaccaggaaaagttttatgtccttgacaacaccaacacagtctgaaagggaaatgacagattctactcctgtctttacagaaacaaatacattagaaaatttgggggcacagaccactgagcacagcagtatccatcaacctggggttcaggaagggctgaccactctcccacgtagtcctgcctctgtctttatggagcagggctctggagaagctgctgccgacccagaaaccaccactgtttcttcattttcattaaacgtagagtatgcaattcaagccgaaaaggaagtagctggcactttgtctccgcatgtggaaactacattctccactgagccaacaggactggttttgagtacagtaatggacagagtagttgctgaaaatataacccaaacatccagggaaatagtgatttcagagcgattaggagaaccaaattatggggcagaaataaggggcttttccacaggttttcctttggaggaagatttcagtggtgactttagagaatactcaacagtgtctcatcccatagcaaaagaagaaacggtaatgatggaaggctctggagatgcagcatttagggacacccagacttcaccatctacagtacctacttcagttcacatcagtcacatatctgactcagaaggacccagtagcaccatggtcagcacttcagccttcccctgggaagagtttacatcctcagctgagggctcaggtgagcaactggtcacagtcagcagctctgttgttccagtgcttcccagtgctgtgcaaaagttttctggtacagcttcctccattatcgacgaaggattgggagaagtgggtactgtcaatgaaattgatagaagatccaccattttaccaacagcagaagtggaaggtacgaaagctccagtagagaaggaggaagtaaaggtcagtggcacagtttcaacaaactttccccaaactatagagccagccaaattatggtctaggcaagaagtcaaccctgtaagacaagaaattgaaagtgaaacaacatcagaggaacaaattcaagaagaaaagtcatttgaatcccctcaaaactctcctgcaacagaacaaacaatctttgattcacagacatttactgaaactgaactcaaaaccacagattattctgtactaacaacaaagaaaacttacagtgatgataaagaaatgaaggaggaagacacttctttagttaacatgtctactccagatccagatgcaaatggcttggaatcttacacaactctccctgaagctactgaaaagtcacattttttcttagctactgcattagtaactgaatctataccagctgaacatgtagtcacagattcaccaatcaaaaaggaagaaagtacaaaacattttccgaaaggcatgagaccaacaattcaagagtcagatactgagctcttattctctggactgggatcaggagaagaagttttacctactctaccaacagagtcagtgaattttactgaagtggaacaaatcaataacacattatatccccacacttctcaagtggaaagtacctcaagtgacaaaattgaagactttaacagaatggaaaatgtggcaaaagaagttggaccactcgtatctcaaacagacatctttgaaggtagtgggtcagtaaccagcacaacattaatagaaattttaagtgacactggagcagaaggacccacggtggcacctctccctttctccacggacatcggacatcctcaaaatcagactgtcaggtgggcagaagaaatccagactagtagaccacaaaccataactgaacaagactctaacaagaattcttcaacagcagaaattaacgaaacaacaacctcatctactgattttctggctagagcttatggttttgaaatggccaaagaatttgttacatcagcaccaaaaccatctgacttgtattatgaaccttctggagaaggatctggagaagtggatattgttgattcatttcacacttctgcaactactcaggcaaccagacaagaaagcagcaccacatttgtttctgatgggtccctggaaaaacatcctgaggtgccaagcgctaaagctgttactgctgatggattcccaacagtttcagtgatgctgcctcttcattcagagcagaacaaaagctcccctgatccaactagcacactgtcaaatacagtgtcatatgagaggtccacagacggtagtttccaagaccgtttcagggaattcgaggattccaccttaaaacctaacagaaaaaaacccactgaaaatattatcatagacctggacaaagaggacaaggatttaatattgacaattacagagagtaccatccttgaaattctacctgagctgacatcggataaaaatactatcatagatattgatcatactaaacctgtgtatgaagacattcttggaatgcaaacagatatagatacagaggtaccatcagaaccacatgacagtaatgatgaaagtaatgatgacagcactcaagttcaagagatctatgaggcagctgtcaacctttctttaactgaggaaacatttgagggctctgctgatgttctggctagctacactcaggcaacacatgatgaatcaatgacttatgaagatagaagccaactagatcacatgggctttcacttcacaactgggatccctgctcctagcacagaaacagaattagacgttttacttcccacggcaacatccctgccaattcctcgtaagtctgccacagttattccagagattgaaggaataaaagctgaagcaaaagccctggatgacatgtttgaatcaagcactttgtctgatggtcaagctattgcagaccaaagtgaaataataccaacattgggccaatttgaaaggactcaggaggagtatgaagacaaaaaacatgctggtccttcttttcagccagaattctcttcaggagctgaggaggcattagtagaccatactccctatctaagtattgctactacccaccttatggatcagagtgtaacagaggtgcctgatgtgatggaaggatccaatcccccatattacactgatacaacattagcagtttcaacatttgcgaagttgtcttctcagacaccatcatctcccctcactatctactcaggcagtgaagcctctggacacacagagatcccccagcccagtgctctgccaggaatagacgtcggctcatctgtaatgtccccacaggattcttttaaggaaattcatgtaaatattgaagcgactttcaaaccatcaagtgaggaataccttcacataactgagcctccctctttatctcctgacacaaaattagaaccttcagaagatgatggtaaacctgagttattagaagaaatggaagcttctcccacagaacttattgctgtggaaggaactgagattctccaagatttccaaaacaaaaccgatggtcaagtttctggagaagcaatcaagatgtttcccaccattaaaacacctgaggctggaactgttattacaactgccgatgaaattgaattagaaggtgctacacagtggccacactctacttctgcttctgccacctatggggtcgaggcaggtgtggtgccttggctaagtccacagacttctgagaggcccacgctttcttcttctccagaaataaaccctgaaactcaagcagctttaatcagagggcaggattccacgatagcagcatcagaacagcaagtggcagcgagaattcttgattccaatgatcaggcaacagtaaaccctgtggaatttaatactgaggttgcaacaccaccattttcccttctggagacttctaatgaaacagatttcctgattggcattaatgaagagtcagtggaaggcacggcaatctatttaccaggacctgatcgctgcaaaatgaacccgtgccttaacggaggcacctgttatcctactgaaacttcctacgtatgcacctgtgtgccaggatacagcggagaccagtgtgaacttgattttgatgaatgtcactctaatccctgtcgtaatggagccacttgtgttgatggttttaacacattcaggtgcctctgccttccaagttatgttggtgcactttgtgagcaagataccgagacatgtgactatggctggcacaaattccaagggcagtgctacaaatactttgcccatcgacgcacatgggatgcagctgaacgggaatgccgtctgcagggtgcccatctcacaagcatcctgtctcacgaagaacaaatgtttgttaatcgtgtgggccatgattatcagtggataggcctcaatgacaagatgtttgagcatgacttccgttggactgatggcagcacactgcaatacgagaattggagacccaaccagccagacagcttcttttctgctggagaagactgtgttgtaatcatttggcatgagaatggccagtggaatgatgttccctgcaattaccatctcacctatacgtgcaagaaaggaacagtcgcttgcggccagccccctgttgtagaaaatgccaagacctttggaaagatgaaacctcgttatgaaatcaactccctgattagataccactgcaaagatggtttcattcaacgtcaccttccaactatccggtgcttaggaaatggaagatgggctatacctaaaattacctgcatgaacccatctgcataccaaaggacttattctatgaaatactttaaaaattcctcatcagcaaaggacaattcaataaatacatccaaacatgatcatcgttggagccggaggtggcaggagtcgaggcgctgatccctaaaatggcgaacatgtgttttcatcatttcagccaaagtcctaacttcctgtgcctttcctatcacctcgagaagtaattatcagttggtttggatttttggaccaccgttcagtcattttgggttgccgtgctcccaaaacattttaaatgaaagtattggcattcaaaaagacagcagacaaaatgaaagaaaatgagagcagaaagtaagcatttccagcctatctaatttctttagttttctatttgcctccagtgcagtccatttcctaatgtataccagcctactgtactatttaaaatgctcaatttcagcaccgatggccatgtaaataagatgatttaatgttgattttaatcctgtatataaaataaaaagtcacaatgagtttgggcatatttaatgatgattatggagccttagaggtctttaatcattggttcggctgcttttatgtagtttaggctggaaatggtttcacttgctctttgactgtcagcaagactgaagatggcttttcctggacagctagaaaacacaaaatcttgtaggtcattgcacctatctcagccataggtgcagtttgcttctacatgatgctaaaggctgcgaatgggatcctgatggaactaaggactccaatgtcgaactcttctttgctgcattcctttttcttcacttacaagaaaggcctgaatggaggacttttctgtaaccaggaacattttttaggggtcaaagtgctaataattaactcaaccaggtctactttttaatggctttcataacactaactcataaggttaccgatcaatgcatttcatacggatatagacctagggctctggagggtgggggattgttaaaacacatgcaaaaaaaaaaaaaaaaaaaaaaaaagaaattttgtatatataaccattttaatcttttataaagttttgaatgttcatgtatgaatgctgcagctgtgaagcatacataaataaatgaagtaagccatactgatttaatttattggatgttattttccctaagacctgaaaatgaacatagtatgctagttatttttcagtgttagccttttactttcctcacacaatttggaatcatataatataggtactttgtccctgattaaataatgtgacggatagaatgcatcaagtgtttattatgaaaagagtggaaaagtatatagcttttagcaaaaggtgtttgcccattctaagaaatgagcgaatatatagaaatagtgtgggcatttcttcctgttaggtggagtgtatgtgttgacatttctccccatctcttcccactctgttttctccccattatttgaataaagtgactgctgaagatgactttgaatccttatccacttaatttaatgtttaaagaaaaacctgtaatggaaagtaagactccttccctaatttcagtttagagcaacttgaagaagagtagacaaaaaataaaatgcacatagaaaaagagaaaaagggcacaaagggattggcccaatattgattctttttttataaaacctcctttggcttagaaggaatgactctagctacaataatacacagtatgtttaagcaggttcccttggttgttgcattaaatgtaatccacctttaggtattttagagcacagaacaacactgtgttgatctagtaggtttctatttttcctttctctttacaatgcacataatactttcctgtatttatatcataacgtgtatagtgtaaaatgtgaatgactttttttgtgaatgaaaatctaaaatctttgtaactttttatatctgcttttgtttcaccaaagaaacctaaaatccttcttttactacac

SEQ ID NO: 4 - protein, 126 aa, Homo sapiens
LHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLT

SEQ ID NO: 5 - protein, 119 aa, Homo sapiens
SPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTV

SEQ ID NO: 6 - protein, 124 aa, Homo sapiens
SGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSR

SEQ ID NO: 7 - protein, 96 aa, Homo sapiens
VVFHYRAATSRYTLNFEAAQKACLDVGAVIATPEQLFAAYEDGFEQCDAGWLADQTVRYPIRAPRVGCYGDKMGKAGVRTYGFRSPQETYDVYCYV

SEQ ID NO: 8 - protein, 95 aa, Homo sapiens
VVFHYRAATSRYTLNFEAAQKACLDVGAVIATPEQLFAAYEDGFEQCDAGWLADQTVRYPIRAPRVGCYGDKMGKAGVRTYGFRSPQETYDVYCY

SEQ ID NO: 9 - protein, 21 aa, Homo sapiens
VVFHYRAATSRYTLNFEAAQK

SEQ ID NO: 10 - protein, 140 aa, Homo sapiens
TPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLL

SEQ ID NO: 11 - protein, 97 aa, Homo sapiens
DVFHLTVPSKFTFEEAAKECENQDARLATVGELQAAWRNGFDQCDYGWLSDASVRHPVTVARAQCGGGLLGVRTLYRFENQTGFPPPDSRFDAYCFK

SEQ ID NO: 12 - protein, 96 aa, Homo sapiens
DVFHLTVPSKFTFEEAAKECENQDARLATVGELQAAWRNGFDQCDYGWLSDASVRHPVTVARAQCGGGLLGVRTLYRFENQTGFPPPDSRFDAYCF

SEQ ID NO: 13 - protein, 151 aa, Homo sapiens
GDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEATTIDLSILAETASPSLSKEPQMVSDRTTPIIPLVDELPVIPTEFPPVGN

SEQ ID NO: 14 - protein, 250 aa, Homo sapiens
ATTIDLSILAETASPSLSKEPQMVSDRTTPIIPLVDELPVIPTEFPPVGNIVSFEQKATVQPQAITDSLATKLPTPTGSTKKPWDMDDYSPSASGPLGKLDISEIKEEVLQSTTGVSHYATDSWDGVVEDKQTQESVTQIEQIEVGPLVTSMEILKHIPSKEFPVTETPLVTARMILESKTEKKMVSTVSELVTTGHYGFTLGEEDDEDRTLTVGSDESTLIFDQIPEVITVSKTSEDTIHTHLEDLESV

SEQ ID NO: 15 - protein, 250 aa, Homo sapiens
SMEILKHIPSKEFPVTETPLVTARMILESKTEKKMVSTVSELVTTGHYGFTLGEEDDEDRTLTVGSDESTLIFDQIPEVITVSKTSEDTIHTHLEDLESVSASTTVSPLIMPDNNGSSMDDWEERQTSGRITEEFLGKYLSTTPFPSQHRTEIELFPYSGDKILVEGISTVIYPSLQTEMTHRRERTETLIPEMRTDTYTDEIQEEITKSPFMGKTEEEVFSGMKLSTSLSEPIHVTESSVEMTKSFDFP

SEQ ID NO: 16 - protein, 180 aa, Homo sapiens
FSGMKLSTSLSEPIHVTESSVEMTKSFDFPTLITKLSAEPTEVRDMEEDFTATPGTTKYDENITTVLLAHGTLSVEAATVSKWSWDEDNTTSKPLESTEPSASSKLPPALLTTVGMNGKDKDIPSFTEDGADEFTLIPDSTQKQLEEVTDEDIAAHGKFTIRFQPTTSTGIAEKSTLRDS

SEQ ID NO: 17 - protein, 175 aa, Homo sapiens
ADEFTLIPDSTQKQLEEVTDEDIAAHGKFTIRFQPTTSTGIAEKSTLRDSTTEEKVPPITSTEGQVYATMEGSALGEVEDVDLSKPVSTVPQFAHTSEVEGLAFVSYSSTQEPTTYVDSSHTIPLSVIPKTDWGVLVPSVPSEDEVLGEPSQDILVIDQTRLEATISPETMRTTK

SEQ ID NO: 18 - protein, 231 aa, Homo sapiens
EGSALGEVEDVDLSKPVSTVPQFAHTSEVEGLAFVSYSSTQEPTTYVDSSHTIPLSVIPKTDWGVLVPSVPSEDEVLGEPSQDILVIDQTRLEATISPETMRTTKITEGTTQEEFPWKEQTAEKPVPALSSTAWTPKEAVTPLDEQEGDGSAYTVSEDELLTGSERVPVLETTPVGKIDHSVSYPPGAVTEHKVKTDEVVTLTPRIGPKVSLSPGPEQKYETEGSSTTGFT

SEQ ID NO: 19 - protein, 277 aa, Homo sapiens
KITEGTTQEEFPWKEQTAEKPVPALSSTAWTPKEAVTPLDEQEGDGSAYTVSEDELLTGSERVPVLETTPVGKIDHSVSYPPGAVTEHKVKTDEVVTLTPRIGPKVSLSPGPEQKYETEGSSTTGFTSSLSPFSTHITQLMEETTTEKTSLEDIDLGSGLFEKPKATELIEFSTIKVTVPSDITTAFSSVDRLHTTSAFKPSSAITKKPPLIDREPGEETTSDMVIIGESTSHVPPTTLEDIVAKETETDIDREYFTTSSPPATQPTRPPTVEDKEA

SEQ ID NO: 20 - protein, 160 aa, Homo sapiens
VTVPSDITTAFSSVDRLHTTSAFKPSSAITKKPPLIDREPGEETTSDMVIIGESTSHVPPTTLEDIVAKETETDIDREYFTTSSPPATQPTRPPTVEDKEAFGPQALSTPQPPASTKFHPDINVYIIEVRENKTGRMSDLSVIGHPIDSESKEDEPCSEE

SEQ ID NO: 21 - protein, 1754 aa, Homo sapiens
RMSDLSVIGHPIDSESKEDEPCSEETDPVHDLMAEILPEFPDIIEIDLYHSEENEEEEEECANATDVTTTPSVQYINGKHLVTTVPKDPEAAEARRGQFESVAPSQNFSDSSESDTHPFVIAKTELSTAVQPNESTETTESLEVTWKPETYPETSEHFSGGEPDVFPTVPFHEEFESGTAKKGAESVTERDTEVGHQAHEHTEPVSLFPEESSGEIAIDQESQKIAFARATEVTFGEEVEKSTSVTYTPTIVPSSASAYVSEEEAVTLIGNPWPDDLLSTKESWVEATPRQVVELSGSSSIPITEGSGEAEEDEDTMFTMVTDLSQRNTTDTLITLDTSRIITESFFEVPATTIYPVSEQPSAKVVPTKFVSETDTSEWISSTTVEEKKRKEEEGTTGTASTFEVYSSTQRSDQLILPFELESPNVATSSDSGTRKSFMSLTTPTQSEREMTDSTPVFTETNTLENLGAQTTEHSSIHQPGVQEGLTTLPRSPASVFMEQGSGEAAADPETTTVSSFSLNVEYAIQAEKEVAGTLSPHVETTFSTEPTGLVLSTVMDRVVAENITQTSREIVISERLGEPNYGAEIRGFSTGFPLEEDFSGDFREYSTVSHPIAKEETVMMEGSGDAAFRDTQTSPSTVPTSVHISHISDSEGPSSTMVSTSAFPWEEFTSSAEGSGEQLVTVSSSVVPVLPSAVQKFSGTASSIIDEGLGEVGTVNEIDRRSTILPTAEVEGTKAPVEKEEVKVSGTVSTNFPQTIEPAKLWSRQEVNPVRQEIESETTSEEQIQEEKSFESPQNSPATEQTIFDSQTFTETELKTTDYSVLTTKKTYSDDKEMKEEDTSLVNMSTPDPDANGLESYTTLPEATEKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTDFLARAYGFEMAKEFVTSAPKPSDLYYEPSGEGSGEVDIVDSFHTSATTQATRQESSTTFVSDGSLEKHPEVPSAKAVTADGFPTVSVMLPLHSEQNKSSPDPTSTLSNTVSYERSTDGSFQDRFREFEDSTLKPNRKKPTENIIIDLDKEDKDLILTITESTILEILPELTSDKNTIIDIDHTKPVYEDILGMQTDIDTEVPSEPHDSNDESNDDSTQVQEIYEAAVNLSLTEETFEGSADVLASYTQATHDESMTYEDRSQLDHMGFHFTTGIPAPSTETELDVLLPTATSLPIPRKSATVIPEIEGIKAEAKALDDMFESSTLSDGQAIADQSEIIPTLGQFERTQEEYEDKKHAGPSFQPEFSSGAEEALVDHTPYLSIATTHLMDQSVTEVPDVMEGSNPPYYTDTTLAVSTFAKLSSQTPSSPLTIYSGSEASGHTEIPQPSALPGIDVGSSVMSPQDSFKEIHVNIEATFKPSSEEYLHITEPPSLSPDTKLEPSEDDGKPELLEEMEASPTELIAVEGTEILQDFQNKTDGQVSGEAIKMFPTIKTPEAGTVITTADEIELEGATQWPHSTSASATYGVEAGVVPWLSPQTSERPTLSSSPEINPETQAALIRGQDSTIAASEQQVAARILDSNDQATVNPVEFNTEVATPPFSLLETSNETDFLIGINEESVEGTAIYLPG

SEQ ID NO: 22 - protein, 540 aa, Homo sapiens
SVIGHPIDSESKEDEPCSEETDPVHDLMAEILPEFPDIIEIDLYHSEENEEEEEECANATDVTTTPSVQYINGKHLVTTVPKDPEAAEARRGQFESVAPSQNFSDSSESDTHPFVIAKTELSTAVQPNESTETTESLEVTWKPETYPETSEHFSGGEPDVFPTVPFHEEFESGTAKKGAESVTERDTEVGHQAHEHTEPVSLFPEESSGEIAIDQESQKIAFARATEVTFGEEVEKSTSVTYTPTIVPSSASAYVSEEEAVTLIGNPWPDDLLSTKESWVEATPRQVVELSGSSSIPITEGSGEAEEDEDTMFTMVTDLSQRNTTDTLITLDTSRIITESFFEVPATTIYPVSEQPSAKVVPTKFVSETDTSEWISSTTVEEKKRKEEEGTTGTASTFEVYSSTQRSDQLILPFELESPNVATSSDSGTRKSFMSLTTPTQSEREMTDSTPVFTETNTLENLGAQTTEHSSIHQPGVQEGLTTLPRSPASVFMEQGSGEAAADPETTTVSSFSLNVEYAIQAEKEVAGTLSPHVETTFST

SEQ ID NO: 23 - protein, 600 aa, Homo sapiens
NLGAQTTEHSSIHQPGVQEGLTTLPRSPASVFMEQGSGEAAADPETTTVSSFSLNVEYAIQAEKEVAGTLSPHVETTFSTEPTGLVLSTVMDRVVAENITQTSREIVISERLGEPNYGAEIRGFSTGFPLEEDFSGDFREYSTVSHPIAKEETVMMEGSGDAAFRDTQTSPSTVPTSVHISHISDSEGPSSTMVSTSAFPWEEFTSSAEGSGEQLVTVSSSVVPVLPSAVQKFSGTASSIIDEGLGEVGTVNEIDRRSTILPTAEVEGTKAPVEKEEVKVSGTVSTNFPQTIEPAKLWSRQEVNPVRQEIESETTSEEQIQEEKSFESPQNSPATEQTIFDSQTFTETELKTTDYSVLTTKKTYSDDKEMKEEDTSLVNMSTPDPDANGLESYTTLPEATEKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTD

SEQ ID NO: 24 - protein, 600 aa, Homo sapiens
EKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTDFLARAYGFEMAKEFVTSAPKPSDLYYEPSGEGSGEVDIVDSFHTSATTQATRQESSTTFVSDGSLEKHPEVPSAKAVTADGFPTVSVMLPLHSEQNKSSPDPTSTLSNTVSYERSTDGSFQDRFREFEDSTLKPNRKKPTENIIIDLDKEDKDLILTITESTILEILPELTSDKNTIIDIDHTKPVYEDILGMQTDIDTEVPSEPHDSNDESNDDSTQVQEIYEAAVNLSLTEETFEGSADVLASYTQATHDESMTYEDRSQLDHMGFHFTTGIPAPSTETELDVLLPTATSLPIPRKSATVIPEIEGIKAEAKALDDMFESSTLSDGQAIADQSEIIPTLGQFERTQEEYEDKKHAGPSFQPEFSSGAEEALVDHTPYLSIATTHLMDQSVTEVPDVME

SEQ ID NO: 25 - protein, 369 aa, Homo sapiens
ESSTLSDGQAIADQSEIIPTLGQFERTQEEYEDKKHAGPSFQPEFSSGAEEALVDHTPYLSIATTHLMDQSVTEVPDVMEGSNPPYYTDTTLAVSTFAKLSSQTPSSPLTIYSGSEASGHTEIPQPSALPGIDVGSSVMSPQDSFKEIHVNIEATFKPSSEEYLHITEPPSLSPDTKLEPSEDDGKPELLEEMEASPTELIAVEGTEILQDFQNKTDGQVSGEAIKMFPTIKTPEAGTVITTADEIELEGATQWPHSTSASATYGVEAGVVPWLSPQTSERPTLSSSPEINPETQAALIRGQDSTIAASEQQVAARILDSNDQATVNPVEFNTEVATPPFSLLETSNETDFLIGINEESVEGTAIYLPG

SEQ ID NO: 26 - protein, 33 aa, Homo sapiens
IKAEAKALDDMFESSTLSDGQAIADQSEIIPTL

SEQ ID NO: 27 - protein, 37 aa, Homo sapiens
GPDRCKMNPCLNGGTCYPTETSYVCTCVPGYSGDQCE

SEQ ID NO: 28 - protein, 35 aa, Homo sapiens
DRCKMNPCLNGGTCYPTETSYVCTCVPGYSGDQCE

SEQ ID NO: 29 - protein, 80 aa, Homo sapiens
GDQCELDFDECHSNPCRNGATCVDGFNTFRCLCLPSYVGALCEQDTETCDYGWHKFQGQCYKYFAHRRTWDAAERECRLQ

SEQ ID NO: 30 - protein, 40 aa, Homo sapiens
EGTAIYLPGPDRCKMNPCLNGGTCYPTETSYVCTCVPGYS

SEQ ID NO: 31 - protein, 30 aa, Homo sapiens
DRCKMNPCLNGGTCYPTETSYVCTCVPGYS

SEQ ID NO: 32 - protein, 37 aa, Homo sapiens
DFDECHSNPCRNGATCVDGFNTFRCLCLPSYVGALCE

SEQ ID NO: 33 - protein, 18 aa, Homo sapiens
DFDECHSNPCRNGATCVD

SEQ ID NO: 34 - protein, 124 aa, Homo sapiens
CDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKG

SEQ ID NO: 35 - protein, 115 aa, Homo sapiens
FQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCK

SEQ ID NO: 36 - protein, 98 aa, Homo sapiens
RRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYH

SEQ ID NO: 37 - protein, 45 aa, Homo sapiens
GHDYQWIGLNDKMFEHDFRWTDGSTLQYENWRPNQPDSFFSAGED

SEQ ID NO: 38 - protein, 25 aa, Homo sapiens
QPDSFFSAGEDCVVIIWHENGQWND

SEQ ID NO: 39 - protein, 61 aa, Homo sapiens
VACGQPPVVENAKTFGKMKPRYEINSLIRYHCKDGFIQRHLPTIRCLGNGRWAIPKITCMN

SEQ ID NO: 40 - protein, 57 aa, Homo sapiens
CGQPPVVENAKTFGKMKPRYEINSLIRYHCKDGFIQRHLPTIRCLGNGRWAIPKITC

SEQ ID NO: 41 - protein, 21 aa, Homo sapiens
KTFGKMKPRYEINSLIRYHCK

SEQ ID NO: 42 - protein, 41 aa, Homo sapiens
SAYQRTYSMKYFKNSSSAKDNSINTSKHDHRWSRRWQESRR

SEQ ID NO: 43 - DNA, 22 bp, Artificial Sequence
  A synthetic oligonucleotide
acaccagaattagaaagttcaa

SEQ ID NO: 44 - DNA, 22 bp, Artificial Sequence
  A synthetic oligonucleotide
agcaccttgtctgatggccaag

SEQ ID NO: 45 - RNA, 9 nt, Artificial Sequence
  A synthetic siRNA
uucaagaga

SEQ ID NO: 46 - DNA, 20 bp, Artificial Sequence
  A synthetic oligonucleotide
ggtcctcagtgtagcccaag

SEQ ID NO: 47 - DNA, 20 bp, Artificial Sequence
  A synthetic oligonucleotide
aatgtgtccgtcgtggatct

SEQ ID NO: 48 - DNA, 24 bp, Artificial Sequence
  A synthetic oligonucleotide
tggggtgagaaccctgtatcgttt

SEQ ID NO: 49 - DNA, 24 bp, Artificial Sequence
  A synthetic oligonucleotide
cccattgatatactgcactgacgg

SEQ ID NO: 50 - DNA, 22 bp, Artificial Sequence
  A synthetic oligonucleotide
gaaaactccatgcttgaagaag

SEQ ID NO: 51 - DNA, 22 bp, Artificial Sequence
  A synthetic oligonucleotide
tgatgtggacttggactcattc

SEQ ID NO: 52 - DNA, 22 bp, Artificial Sequence
  A synthetic oligonucleotide
cccctgtgaaaataagagcaag

SEQ ID NO: 53 - DNA, 24 bp, Artificial Sequence
  A synthetic oligonucleotide
gcttctatgcagttgatgaagatg

SEQ ID NO: 54 - DNA, 22 bp, Artificial Sequence
  A synthetic oligonucleotide
tgtgtaatgaaagacggcacac

SEQ ID NO: 55 - DNA, 22 bp, Artificial Sequence
  A synthetic oligonucleotide
tcaaactccactttgctcttga

SEQ ID NO: 56 - DNA, 22 bp, Artificial Sequence
  A synthetic oligonucleotide
agacaaagccagagtccttcag

SEQ ID NO: 57 - DNA, 22 bp, Artificial Sequence
  A synthetic oligonucleotide
ttaggagagcattggaaattgg

SEQ ID NO: 58 - DNA, 22 bp, Artificial Sequence
  A synthetic oligonucleotide
gtctactgaacttcggggtgat

SEQ ID NO: 59 - DNA, 24 bp, Artificial Sequence
  A synthetic oligonucleotide
ttgagaagatgatctgagtgtgag

SEQ ID NO: 60 - DNA, 24 bp, Artificial Sequence
  A synthetic oligonucleotide
cagctacctgctgggaaggaagaa

SEQ ID NO: 61 - DNA, 22 bp, Artificial Sequence
  A synthetic oligonucleotide
ccaagagttgggttcacttcca

SEQ ID NO: 62 - DNA, 24 bp, Artificial Sequence
  A synthetic oligonucleotide
agcaaccctgtattatgtatttct

SEQ ID NO: 63 - DNA, 24 bp, Artificial Sequence
  A synthetic oligonucleotide
atcgacttgggatccagaaggtga

SEQ ID NO: 64 - DNA, 24 bp, Artificial Sequence
  A synthetic oligonucleotide
acctaccgcacccgagatggtcag

SEQ ID NO: 65 - DNA, 24 bp, Artificial Sequence
  A synthetic oligonucleotide
ctgctgccagaaacttcggaaggg

SEQ ID NO: 66 - DNA, 24 bp, Artificial Sequence
  A synthetic oligonucleotide
cttcaacagcgacacccactcctc

SEQ ID NO: 67 - DNA, 22 bp, Artificial Sequence
  A synthetic oligonucleotide
gtccaccaccctgttgctgtag

SEQ ID NO: 68 - DNA, 24 bp, Artificial Sequence
  A synthetic oligonucleotide
gagtcagtggaaggcacggcaatc

SEQ ID NO: 69 - DNA, 23 bp, Artificial Sequence
  A synthetic oligonucleotide
tggtctccgctgtatcctggcac

SEQ ID NO: 70 - DNA, 20 bp, Artificial Sequence
  A synthetic oligonucleotide
tgcccagaaaatgaaaaagg

SEQ ID NO: 71 - DNA, 20 bp, Artificial Sequence
  A synthetic oligonucleotide
gtgtatgtggcaatgcgttc

SEQ ID NO: 72 - DNA, 20 bp, Artificial Sequence
  A synthetic oligonucleotide
cctccctgtcagatgaggac

SEQ ID NO: 73 - DNA, 20 bp, Artificial Sequence
  A synthetic oligonucleotide
ccaggctgaggtattccttg

SEQ ID NO: 74 - DNA, 20 bp, Artificial Sequence
  A synthetic oligonucleotide
tctacgaggaggagatgcgg

SEQ ID NO: 75 - DNA, 20 bp, Artificial Sequence
  A synthetic oligonucleotide
ggtcaagacgtgccagagac

SEQ ID NO: 76 - DNA, 21 bp, Artificial Sequence
  A synthetic oligonucleotide
tccaatggcaaagtgaatgac

SEQ ID NO: 77 - DNA, 21 bp, Artificial Sequence
  A synthetic oligonucleotide
ctttgcaggtgctctttttga

(19) United States
(12) Patent Application Publication
(10) Pub. No.: US 2015/0017091 A1
(43) Pub. Date: Jan. 15, 2015

(54) DETECTION AND TREATMENT OF METASTATIC DISEASE

(71) Applicant: CORNELL UNIVERSITY, Ithaca, NY (US)

(72) Inventors: Vivek Mittal, Greenlawn, NY (US); Ding Cheng Gao, New York, NY (US)

(21) Appl. No.: 14/239,245

(22) PCT Filed: Aug. 16, 2012
(86) PCT No.: PCT/US2012/051207

§ 371 Date: Oct. 7, 2014

Related U.S. Application Data
(60) Provisional application No. 61/525,007, filed on Aug. 18, 2011.

(51) Int. Cl. A61K 31/7088; A61K 39/395; C12N 15/113; A61K 45/06; C07K 16/18; G01N 33/574

(52) U.S. Cl. CPC A61K 31/7088; C07K 16/18; G01N 33/57407; C12N 15/113; A61K 45/06; A61K 39/3955
USPC 424/1.11; 424/9.1; 424/450; 424/139.1; 435/7.23; 514/19.3; 514/44 A

(57) ABSTRACT

The establishment and growth of metastatic tumors can be detected and inhibited by the methods and compositions described herein. As illustrated herein, agents that inhibit the expression or activity of versican, for example, in bone marrow cells effectively halt the growth and establishment of metastatic tumors at distal sites from a primary tumor site. In general, the primary tumor is unaffected by versican inhibitors but metastasis is substantially eliminated.

This application claims benefit of the filing date of U.S. Provisional Patent Application No. 61/525,007, filed Aug. 18, 2011, the contents of which is specifically incorporated herein in its entirety.

### BACKGROUND

Malignant primary tumor cells colonize distal target organs to form micrometastases and in some cases, the micrometastases progress to lethal macrometastases (Townson & Chambers, Cell Cycle 5: 1744-1750 (2006); Naumov et al., J Natl Cancer Inst 98: 316-325 (2006)). As a consequence, such metastasis results in more than 90% of human cancer-related deaths (Gupta & Massague, Cell 127: 679-695 (2006)). Rather than dying from problems relating to the primary tumor, most patients die from extensive metastatic tumor growth.

Further information on the molecular mechanisms and the extrinsic microenvironmental factors that enhance the metastatic potential of primary tumor cells, and of micrometastatic tumor cells at distal sites, would facilitate development of more effective cancer treatments.

### SUMMARY OF THE INVENTION

Methods and compositions are described herein for detecting and/or inhibiting the establishment and growth of metastatic tumors. Such methods and compositions can inhibit the expression or activity of versican in cells, and by doing so the inhibitors inhibit the metastasis promoting functions provided by myeloid progenitor cells and descendants of myeloid progenitor cells.

One aspect of the invention is a method of inhibiting establishment or growth of metastatic tumor cells at a site distal from a primary tumor in an animal comprising administering to the animal a composition comprising a versican inhibitor to thereby inhibit establishment or growth of metastatic tumor cells at a site distal from a primary tumor in the animal.

In some embodiments, the versican inhibitor may not affect growth of the primary tumor or the animal has undergone surgery to remove the primary tumor. In some embodiments, the versican inhibitor is administered to bone marrow or to a site that can have metastatic tumor cells. The versican inhibitor can inhibit versican expression in bone marrow cells, bone marrow-derived cells or myeloid progenitor cells of the animal. For example, the versican inhibitor is formulated to target bone marrow, bone marrow-derived cells or myeloid progenitor cells.

Such methods can inhibit recruitment of myeloid progenitor cells to a premetastatic or metastatic site in the animal. Such methods can also inhibit TGF-β/Smad2/3 signaling in the animal.

Versican inhibitors used in the methods and compositions described herein can include selected from the group consisting of budesonide, one or more hyaluronan oligomers, one or more anti-versican antibodies, one or more non-functioning versican peptides, one or more versican inhibitory nucleic acids, and combinations thereof. Versican inhibitory nucleic acids used in the methods and compositions described herein can specifically bind to a versican mRNA under physiological conditions and can inhibit expression or translation of a versican mRNA. For example, versican inhibitory nucleic acids used in the methods and compositions described herein can include at least one versican inhibitory nucleic acid with:
a sequence comprising 5′-ACACCAGAATTAG AAAGTTCAA-3′ (shVcn1; SEQ ID NO:43), or 5′-AGCACCTTGTCTGATGGCCAAG-3′ (shVcn2; SEQ ID NO:44);
an RNA sequence corresponding to a DNA sequence comprising 5′-ACACCAGAATTAG AAAGTTCAA-3′ (shVcn1; SEQ ID NO:43), or 5′-AGCACCTTGTCTGATGGCCAAG-3′ (shVcn2; SEQ ID NO:44);
a DNA or RNA sequence comprising a sequence complementary to 5′-ACACCAGAATTAGAAAGTTCAA-3′ (shVcn1; SEQ ID NO:43), or 5′-AGCACCTTGTCTGATGGCCAAG-3′ (shVcn2; SEQ ID NO:44); or
a combination thereof.

Compositions and methods described herein can include at least one versican inhibitory peptide, for example, a peptide with a sequence that has at least 90% sequence identity to a sequence selected from the group consisting of SEQ ID NO:4-41 and 42.

Compositions that include versican inhibitors can also include an antibody that specifically binds to CD11b, CD33, VEGF receptor, AFP, CEA, CA-125, MUC-1, ETA, tyrosinase, ras, p53, MAGE1, or combinations of antibodies that specifically bind to any of CD11b, CD33, VEGF receptor, AFP, CEA, CA-125, MUC-1, ETA, tyrosinase, ras, p53, and MAGE1.

Compositions and methods described herein can include an additional therapeutic agent or anti-cancer agent, for example, an agent selected from the group consisting of a radioactive drug, topoisomerase inhibitor, DNA binding agent, anti-metabolite, cytoskeletal-interacting drug, ionizing radiation, or a combination thereof.

Compositions that include versican inhibitors can also include cholesterol, phospholipids, mannose, retinal, a fat soluble vitamin, polyethylene glycol, technetium-99m (^{99m}Tc), hemoglobin, or a combination thereof.

Compositions that include versican inhibitors can be formulated as a liposomal formulation. For example, compositions formulated as a liposomal formulation can have liposomes that comprise non-polymer molecules embedded within the liposomal exterior or bound to the exterior of the liposome, wherein the non-polymer molecules bind to a receptor or cell-membrane protein on the surface of a bone marrow cell, a bone marrow-derived cell, a myeloid progenitor cell, or a metastatic tumor cell. In some embodiments, compositions formulated as a liposomal formulation can have liposomes that include non-polymer molecules embedded within the liposomal exterior or bound to the exterior of the liposome, wherein the non-polymer molecules are selected from the group consisting of haptens, enzymes, antibodies, antibody fragments, cytokines, hormones, peptides, polypeptides, proteins or a combination thereof.

The treatment methods described herein can also include detecting whether the animal has a metastatic tumor. For example, detection that the animal has a metastatic tumor can include testing whether a test sample from the animal expresses at least two-fold higher levels of versican than a negative control sample. For example, the negative control sample can be a non-metastatic sample of the same tissue-type or fluid type as the test sample. The test sample can be a tissue sample or a bodily fluid.

Another aspect of the invention is a method of detecting whether an animal has at least one metastatic tumor that includes:
(a) measuring versican expression levels in a test sample from the animal; and
(b) detecting that the animal has at least one metastatic tumor when at least two-fold higher levels of versican are expressed in the test sample than in a negative control sample.
   For example, the negative control sample can be a non-metastatic sample of the same tissue-type or fluid type as the test sample. The test sample can be a tissue sample or a bodily fluid. Such a detection method can include administering an anti-cancer agent to the animal when at least two-fold higher levels of versican are expressed in the test sample than in a negative control sample.
   For example, a versican inhibitor can be administered to the animal when at least two-fold higher levels of versican are expressed in the test sample than in a negative control sample.

### DESCRIPTION OF THE FIGURES

FIG. 1A-1E illustrates that myeloid cells are recruited to the lung in MMTV-PyMT mice. FIG. 1A shows flow cytometric plots illustrating increased recruitment of bone marrow-derived (GFP⁺) CD11b⁺Gr1⁺ myeloid cells in the lungs of MMTV-PyMT mice (12 weeks old; FIG. 1A2) compared with wild type (WT; FIG. 1A1) mice. Representative plots were derived from 3 independent experiments. BM, bone marrow. FIG. 1B, immunostaining showing increased recruitment of bone marrow-derived GFP⁺Gr1⁺ cells in the lungs of MMTV-PyMT mice compared with WT mice. DAPI, 4′,6-diamidino-2-phenylindole. The lighter areas exhibit fluorescence from cells expressing GFP and/or Gr1. FIG. 1C graphically illustrates the recruitment of CD11b⁺Gr1⁺ myeloid cells in the lungs of MMTVPyMT mice as a function of the age of the mice in weeks. The numbers of CD11b⁺Gr1⁺ cells were normalized to 1×10⁵ total lung cells analysed per animal. Mean±SD. The symbol*means P<0.01 as compared with WT mice of same age. FIG. 1D shows that the majority of myeloid cells are Gr1⁺F4/80⁻ in the metastatic lung but are Gr1⁻F4/80⁺ macrophages in the primary tumors. FIG. 1E shows the kinetics of recruitment of the myeloid CD11b⁺Gr1⁺ progenitor cells to the lungs of MMTV-PyMT mice over time as a function of metastases formation. Increased recruitment of the BM myeloid cells (GR1 staining; lighter areas) was observed at week 8-10 when no metastatic foci (stained with anti-PyMT antibody; lighter areas visible at 12 weeks) were detectable. H&E staining of the lungs are shown at the right.

FIG. 2A-2L illustrates that versican is expressed by tumor-elicited CD11b⁺Ly6C^{high} myeloid cells in the metastatic lung. FIG. 2A graphically illustrates versican (Vcn) expression levels in Gr1⁺ myeloid cells (CD11b⁺Gr1⁺), in Gr1⁻ stromal cells (CD11b⁻Gr1⁻), in T cells (CD3⁺), and in B cells (B220⁺) that were sorted from wild type (WT) or metastatic lung (ML) tissues (representative data from two individual experiments). Expression levels were quantified by quantitative RT-PCR analysis. Versican expression was normalized to an internal control (GAPDH; glyceraldehyde-3-phosphate dehydrogenase). The relative expression level of the different cell types is shown as compared to wild type lung, *p<0.01 as indicated. FIG. 2B shows a Western blot illustrating versican levels in the lungs from MMTV-PyMT mice and control mice. β-actin served as an internal control. FIG. 2C shows a series of flow cytometry plots, where the top left panel shows that CD11b⁺ cells are present in the metastatic lung of MMTV-PyMT mice (10-week old). The top right panel shows that the gated CD11b+ cells can be sorted into CD11b⁺Ly6G^{high} and CD11b⁺Ly6C^{high} cells. The two lower panels are flow cytometry plots showing the purity of the post-sorted CD 11b⁺Ly6G^{high} and CD11b⁺Ly6C^{high} cells. FIG. 2D graphically illustrates quantitative RT-PCR detection of versican expression levels in sorted CD11b⁺Ly6G^{high} and CD11b⁺Ly6C^{high} cells compared to the total cells from metastatic lungs. GAPDH was used as internal control. Representative data is from three independent experiments. *p<0.01 as indicated. FIG. 2E illustrates versican expression as detected by immunohistochemical staining with anti-versican antibodies. Hematoxylin (Hem) was used to determine the morphology of the nucleus. Versican protein was detected in the mononuclear CD11b⁺Ly6C^{high} cells. FIG. 2F shows a Western blot illustrating versican levels in flow sorted CD11b⁺Ly6G^{high} and CD11b⁺Ly6C^{high} cells from metastatic lungs of MMTV-PyMT mice. β-actin served as an internal control. FIG. 2G illustrates recruitment CD11b⁺Ly6G^{high} myeloid cells while FIG. 2H illustrates recruitment CD11b⁺Ly6C^{high} myeloid cells in the lung of MMTV-PyMT mice. The numbers of myeloid cells for FIGS. 2G and 2H were normalized to 1×10⁵ total lung cells. n=3 per group *p<0.01 as compared to WT mice of same age. FIG. 2I graphically illustrates versican expression levels in the primary tumor and metastatic lungs of MMTVPyMT mice. FIG. 2J graphically illustrates versican expression as detected by quantitative RT-PCR with GAPDH expression as an internal control, n==3. Total cells (Tot), sorted Ly6Chigh (6C), Ly6Ghigh (6G), endothelial cells (EC), fibroblasts (Fibre) and all marker negative cells (All-) were sorted from the lung of MMTV-PyMT mice. FIG. 2K shows flow cytometry analysis of fibroblasts in the metastatic lung of MMTV-PyMT mice as compared with wild type mice (WT). The numbers indicate the percentage of each cell subtype in the total lung cells. The plot shows the quantification of the recruited fibroblast in the metastatic lung as compared with that of Ly6Chigh cells. FIG. 2L graphically illustrates versican expression in tumor cell lines including breast cancer cell lines MCF7, MDA-MB-231, and LM2; prostate cancer cell lines PC3 and LN4; colon cancer cell lines SW480 and SW640; and flow cytometry sorted lung tumor cells Epcam+) and myeloid cells (CD11b+) from the lung of cancer patients and a lung cancer cell line A549. Versican expression levels were determined by quantitative RT-PCR analysis with GAPDH used as an internal control. The numbers indicate the relative versican expression levels in the indicated cell types as compared to the versican expression in MDA-MB-231 cells.

FIG. 3A-3P illustrate that versican (Vcn) deficiency in myeloid cells impairs macrometastases in MMTV-PyMT mice. FIG. 3A is a schematic diagram of Versican isoforms (V1, V2, and V3) showing the identity of versican V1 mRNA (NM_{—}019389.2) nucleotide positions that correspond to the short hairpin RNA sequences (shVcn1 with 5′-ACACCAGAATTAG AAAGTTCAA-3′; SEQ ID NO:43 and shVcn2 with 5′-AGCACCTTGTCTGATGGCCAAG-3′; SEQ ID NO:44) that are identified as short bars above and below exon 8 and that were used to inhibit versican expression in vivo. FIG. 3B illustrates that Gr1⁺ cells express versican isoform 1 (V1) but not isoform 2 (V2). CD11b⁺Gr1⁺ cells were sorted from the lungs of tumor bearing animals by flow cytometry and analyzed for versican isoform expression by RT-PCR. 3T3 cells served as positive control. FIG. 3C shows a Western blot illustrating reduction in versican protein levels by versican shRNAs (shVcn1 and shVcn2) compared to the control non-specific shRNA (shNS). β-actin serves as an internal control. FIG. 3D illustrates the percentage of GFP⁺ cells as analyzed by flow cytometry in total peripheral blood cells from control wild type mice (FIG. 3D-1 and FIG. 3D-2), β-actin-GFP transgenic mice (FIG. 3D-3 and FIG. 3D-4), shNS-GFP⁺ bone marrow transplanted mice (FIG. 3D-3) and shVcn-GFP⁺ bone marrow transplanted mice (FIG. 3D-4) at 4 weeks after bone marrow transplantation. As shown, the percentage of GFP⁺ cells detected in bone marrow transplanted mice is comparable to that of β-actin-GFP transgenic mice, indicating full reconstitution of bone marrow cells and that the shRNAs did not significantly affect the number or distribution of bone marrow cells. FIG. 3E graphically illustrates versican expression in the bone marrow of wild type mice (WT) compared to MMTVPyMT mice transplanted with control shNS-bone marrow, versican knockdown shVcn1-bone marrow and shVcn2-bone marrow. The mice were 10-weeks old when versican expression was detected by quantitative RT-PCR. Versican expression was normalized to the internal control (GAPDH). The relative versican expression level as compared to WT BM is shown, *p<0.01, n=3 per group. FIG. 3F illustrates that versican (Vcn) deficiency in myeloid cells impairs macrometastases in MMTV-PyMT mice as shown by representative microscopy images where versican expression in lung cells is the lightest shade (green in the original) and Gr1⁺ expression is the medium shade (red in the original) of shVcn-bone marrow transplant MMTV-PyMT mice compared with shNS-bone marrow transplant mice (10 weeks old). The cells were also DAPI (40,6-diamidino-2-phenylindole) stained. As shown, far fewer cells express versican when the shVcn inhibitory RNA is expressed. FIG. 3G shows a western blot of versican expression in lung tissues from control shNS-bone marrow transplant and shVcn-bone marrow transplant MMTV-PyMT mice. FIG. 3H shows representative lung images (stained with anti-PyMT antibody) from MMTV-PyMT mice (15 weeks old) that received either shNS-bone marrow transplants (left panel) or shVcn-bone marrow transplants (right panel). Arrows mark pulmonary metastases. Scale bar, 2 mm. FIG. 3I graphically illustrates the percentage of metastatic tumor area in the lungs of M MTV-PyMT mice who received either shNS-bone marrow transplants or shVcn-bone marrow transplants (mice were 15 weeks old, n=7-9 per group; *, P<0.01 as compared with shNS-bone marrow transplant group). FIG. 3J graphically illustrates the number of metastases in MMTV-PyMT mice who received either shNS-bone marrow transplants or shVcn-bone marrow transplants. The average number of micrometastases (<1 mm in diameter) and macrometastases (>1 mm in diameter) were counted from at least 5 sections from individual animals, n=7-9; *, P<0.01 as compared with shNS-bone marrow transplant group. FIG. 3K shows proliferating cells in a micrometastasis within the lung of a shNS-bone marrow transplant mouse as compared with a micrometastasis within the lung of a shVcn-bone marrow transplant mouse. Cell proliferation was detected by staining tissues with Ki67 (lighter areas; magenta in the original). FIG. 3L graphically illustrates the ratio of proliferation in micrometastases from the lungs of shNS-bone marrow transplant mice as compared with micrometastases from the lungs of a shVcn-bone marrow transplant mice. As shown, less proliferating cells were present in lesions from shVcn-bone marrow transplant mice compared with shNS-bone marrow transplant mice. n=10; *, P<0.01. FIG. 3M shows images of a micrometastasis (FIG. 3M-1) and a macrometastasis (FIG. 3M-2) within the lungs of MMTVPyMT mice as detected by immunohistochemistry. Lung sections were taken from control shNS and shVcn bone marrow transplanted MMTV-PyMT mice that were stained with anti-PyMT antibodies to identify the metastatic cells (dark areas; brown in the original). FIG. 3N illustrates recruitment of B cells (B220⁺), T cells (CD3⁺) and CD11b⁺Gr1⁺ cells in the lungs of control shNS-bone marrow transplant (upper two panels) and shVcn-bone marrow transplant (lower two panels) MMTV-PyMT mice. The numbers indicate the percentage of each subtype cell in the total lung cells collected. Representative scatter plots of each group are shown (n=7-10). FIG. 3O graphically illustrates the number of CD11b⁺Gr1⁺ cells, B cells (B220⁺), and T cells (CD3⁺) in the lungs of shNS-bone marrow transplant (dark bars) and shVcn-bone marrow transplant (hatched bars) MMTV-PyMT mice. Numbers of cell subtypes were normalized to 1×10⁵ total lung cells. (n=7-10 per group). FIG. 3P graphically illustrates expression levels of the genes listed along the x-axis in shNS-bone marrow transplant (cont-BMT; dark bars) and shVcn-bone marrow transplant (sh-Vcn-BMT; light bars) MMTV-PyMT mice. Expression levels of Arginase 1 (Arg 1), Arginase 2 (Arg2), NO synthase 2 (Nos2), interleukin (IL), and tumor necrotic factor α (TNFα) were quantified using quantitative RT-PCR. The results of FIG. 3A-3P indicate that while versican deficiency in myeloid cells impairs macrometastases in MMTV-PyMT mice, versican knockdown does not perturb the recruitment of bone marrow-derived cells nor does it alter the immune microenvironment in the lungs of MMTV-PyMT mice.

FIG. 4A-4I show that versican enhances proliferation and induces mesenchymal-to-epithelial transition (MET) in metastatic tumor cells. FIG. 4A shows that there are abundant proliferating cells in the macrometastatic lesion in lungs of mice transplanted with bone marrow that express the control shNS short hairpin RNA, as detected by immunofluorescent staining of Ki67 (lighter areas; magenta in the original). As illustrated expression of the control shNS does not inhibit metastatic cell growth. FIG. 4B illustrates that addition of secreted versican V1 (right panel) to the culture media enhanced cell proliferation in MDA-MB-231 cells as determined by EdU staining and flow cytometry, compared to MDA-MB-231 cells that received no versican. The numbers indicate the percentage of cells in different phases of a cell cycle, where the S phase cell cluster is at the top, the G0+G1 phase cells cluster closest to the origin and the M+G2 phase cells cluster at the lower right. Representative cell flow cytometry plots from 3 independent experiments are shown. FIG. 4C illustrates the numbers or percentage of metastatic breast cancer MDA-MB-231 cells in various cell cycle phases after treatment with CD11b⁺Gr1⁺ conditioned media (CM) or control media (Cont). Flow cytometric plots in the left two panels illustrate the relative number of cells in S phase (upper cluster) after treatment with CD11b⁺Gr1⁺ conditioned media (CM) or control media (Cont). The graph to the right shows that more MDA-MB-231 breast cancer cells are in S-phase cells after treatment with CD11b⁺Gr1⁺ conditioned media than after treatment with control media. Representative plots are from 3 experiments. FIG. 4D graphically compares epithelial/mesenchymal marker expression in MDA-MB-231 cells treated with CD11b⁺Gr1⁺ conditioned media, control media or biochemically purified versican (+Vcn, 2.5 mg/mL) as assessed by quantitative RT-PCR analysis. n=3; *, P<0.01 as compared with untreated cells (Cont). Vcn, versican; E-cad, E-cadherin; Occl, occludin; Vim, vimentin. FIG. 4E shows microscopic images of MDA-MB-231 cells that were exposed to the secreted form of versican (V1 isoform) (MDA-Vcn) and MDA-MB-231 control cells that were not exposed to versican (MDA (Cont)). The images illustrate the morphology (‘phase’; top two panels) and expression of the following epithelial/mesenchymal markers: E-cad (E-cadherin; middle two panels) and Vim (vimentin; lower two panels). Lighter areas are areas of marker expression. FIG. 4F shows Western blots illustrating expression of versican (>250 kDa), as well as epithelial to mesenchymal transition markers, phospho(p)-Smad2, and total Smad2/3expression in MDA-MB-231 cells that were exposed to the secreted form of versican (V1 isoform) (MDA-Vcn) and MDA-MB-231 control cells that were not exposed to versican (MDA (Cont)) cells. Representative data from 3 experiments are shown. FIG. 4G shows a Western blot illustrating that a specific versican band (at about 250 kDa) is detected after chondroitinase ABC (Chon) treatment of CD11b⁺Gr1⁺ conditioned media and removal of debris. SN, supernatant. FIG. 4H shows a silver stained gel where versican is visible from supernatants before and after purification with Ni-NTA columns via the 6×His tag on the versican protein. M, Marker; SN, supernatant before purification; FT, flow through; W, wash; and E, elution. A band of about 250 kDa was observed in the elute indicating that the versican was substantially purified. FIG. 4I graphically illustrates expression levels of several genes in MDA-MB-231 control (Cont) and MDA-MB-231 versican-treated (Vcn) cells as determined by RT-PCR. The relative expression of the following epithelial and mesenchymal markers was assessed: E-cadherin, occludin, vimentin and snail. Expression levels were normalized to MDA-MB-231 control cells with GAPDH as internal control. *p<0.01 as compared with MDA-MB-231 control cells.

FIG. 5A-5I illustrate that mesenchymal to epithelial transition occurs in metastases formation with MDA-MB-231 cells in vivo. FIG. 5A shows lung tissue from animals injected with MDA-MB-231 breast cancer cells (4 weeks after inoculation) where the metastases were detected by staining with anti-human pan cytokeratin antibody (Hu-Keratin) and analysed for human E-cadherin (E-cad) expression. The lighter areas are areas of immunostaining. FIG. 5B graphically illustrates E-cadherin expression levels as detected by quantitative RT-PCR in MDA-MB-231 cells cultured in vitro (cultured) and sorted back from SCID mice 4 weeks after tail vein injection (mets). GAPDH served as internal expression control, *p<0.01. FIG. 5C illustrates that mesenchymal to epithelial transition occurs during metastases formation in vivo, as illustrated by E-cadherin and vimentin expression in lung metastases of breast cancer patients. Representative immunofluorescent images showing E-cadherin (left) is highly expressed by tumor cells while vimentin (right) is not expressed by tumor cells. However, vimentin is expressed by the adjacent stroma (lighter areas outside the area enclosed by the dashed line). The dashed line indicates the border of metastatic tumor cells and stroma, (n=5). FIG. 5D provides bioluminescent images (BLI) showing that depletion of versican-producing CD11b⁺Gr1⁺ cells by anti-Gr1 antibody treatment (bottom panel) inhibited lung metastases formed by MDA-MB-231 cells. No such inhibition was observed in IgG-treated control animals (top panel). The dark areas indicate the size of the area of versican-producing CD11b⁺Gr1⁺ cells. Scale bar depicts the photon flux (photons/sec) where lighter areas in the center of dark areas indicate tumor marker expression. FIG. 5E graphically illustrates the amount of pulmonary metastases as detected by BLI at days 0, 7, 14, 21, 28, and 35 after inoculation of MDA-MB-231 breast cancer cells with (square symbols) and without (triangle symbols) anti-Gr1 treatment. The relative BLI areas were normalized using the values from day 0. n=10; *, P<0.01. FIG. 5F graphically illustrates versican expression levels in myeloid cells harvested from wild-type (WT). control antibody-treated (Cont-IgG) or anti-Gr1 antibody-treated (Anti-Gr1) tumor-bearing animals (+Tum). Versican expression was analysed by RT-PCR (n=3 in each group) using GAPDH (glyceraldehyde-3-phosphate dehydrogenase) expression as an internal control for RT-PCR. *, P<0.01 as indicated. FIG. 5G shows images illustrating expression of E-cadherin (lighter areas; magenta in the original) and vimentin (lighter areas; green in the original) in pulmonary metastases formed by MDA-MB-231 cells in mice treated with control IgG or anti-Gr1 antibodies. Tumor cells were detected by the intrinsic RFP signal (lighter areas; red in the original). The sizes of the metastatic lesions are shown within the dotted lines. Note that lung epithelial cells surrounding the metastases also stain for E-cadherin (lighter areas). FIG. 5H shows that versican promotes lung metastasis in vivo as detected by representative BLIs illustrating accelerated metastases of MDA-MB-231 cells that express versican (MDA-Vcn) in the lung after tail vein injection. As shown, the versican-expressing MDA-Vcn cells give rise to larger (dark) areas of metastasis compared to control MDA-MB-231 cells that do not express versican (n=5). Lighter areas in the center of the dark BLI areas indicate enhanced tumor marker expression. FIG. 5I graphically illustrates the extent of pulmonary metastases as quantified by BLI at days 0, 7, 14, 21, 28, and 35 after inoculation of MDA-MB-231 cells that express versican (MDA-Vcn) or control MDA-MB-231 cells that do not express versican. The relative BLIs are normalized using the values from day 0. n=5; *, P<0.01.

FIG. 6A-6E illustrate versican expression in the metastatic tumors of patients with breast cancer. FIG. 6A shows representative images of lungs from normal healthy subjects (Normal Lung, n=5) and patients with breast cancer with metastases (Lung Mets, n=11) showing darker stromal versican expression as detected by immunohistochemistry. Scale bar, 200 mm. FIG. 6B shows lung metastases from a patient with breast cancer showing colocalization of recruited CD11b⁺ (dark areas; brown in the original) myeloid cells with versican (lighter areas; red in the original) as shown by immunohistochemistry. FIG. 6C graphically illustrates versican expression levels in lung metastases (n=6) and liver metastases (n=11) of patients with breast cancer as compared with healthy normal (norm) tissues (n=5 and n=4, respectively). Expression levels were quantified by RT-PCR. *, p<0.01. FIG. 6D shows that CD11b⁺ cells in the human metastatic lungs are composed of CD11b⁺CD33+ (upper right cluster) and CD11b⁺CD33⁻ (upper left cluster) populations as detected by flow cytometry. FIG. 6E graphically illustrates versican expression levels in sorted total cells (Tot), tumor cells (EpCam⁺), CD11b⁺CD33⁺ myeloid cells, and CD11b⁺CD33⁺ cells in a patient with breast cancer and lung metastases.

FIG. 7 is a schematic diagram of a model illustrating the contribution of bone marrow-derived myeloid cells to the formation of metastases. EMT, epithelial to mesenchymal transition; MET, mesenchymal to epithelial transition; BM, bone marrow.

### DETAILED DESCRIPTION

As described herein, the establishment and growth of metastatic tumors are inhibited by inhibiting the expression or activity of versican. Hence, the present invention solves a longstanding problem that when a primary tumor is detected, micrometastases at distal sites may already have been established but may not readily be detected. Treatment delays can result. However, versican is a secreted protein. Thus, detection of heightened versican expression levels can be used to evaluate whether metastasis has or is occurring. By administering versican inhibitors, the establishment and growth of micrometastases are effectively terminated so that even small metastatic sites are treated, further metastases are halted, and small metastatic tumors no longer progress into larger tumors.

Versican inhibition does not adversely affect normal cell production or function, and does not affect the primary site tumor. While not limiting the scope of the invention, it is hypothesized that versican inhibitors inhibit tumor-elicited support functions provided by bone marrow (BM)-derived progenitor cells that would otherwise contribute significantly to the stroma of metastatic sites. By inhibiting versican, for example within the bone marrow or in bone marrow-derived progenitor cells, the outgrowth of disseminated tumor cells is inhibited as well as the support functions that contribute to the establishment of metastases and angiogenesis mediated progression of micrometastases to macrometastases.

In some embodiments, the versican inhibitors are targeted to the bone marrow and not to the primary tumor site or to known metastatic sites. In some embodiments, the versican inhibitors are targeted to the bone marrow-derived cells (e.g., myeloid progenitor cells) and not to the primary tumor site or to known metastatic sites. Hence, treatment of metastasis pursuant to the methods provided herein can include administration of versican inhibitors to the bone marrow or administration of versican inhibitors formulated to target bone marrow-derived cells, or myeloid progenitor cells.

Further information regarding the invention is provided in the description below, as well as in the Examples, figures, statements describing aspects of the invention and in the claims.

### Metastasis

Activation of epithelial to mesenchymal transition (EMT) is a developmental program that also endows metastatic properties upon cancer cells to promote invasion, migration, and subsequent tumor cell dissemination (Polyak & Weinberg, Nat Rev Cancer 9:265-73 (2009)). Following dissemination, establishment of metastatic lesions depends on the organ-colonizing properties of disseminated tumor cells as well as on permissive conditions or the “metastatic niche” that may be present in the microenvironment of target organs (Fidler, Nat Rev Cancer 3:453-8 (2003); Gupta & Massague, Cell 127: 679-95 (2006)).

Tumor-associated myeloid cells are believed to promote tumor development by stimulating tumor growth, angiogenesis, invasion, and metastasis. As is known to those of skill in the art, myeloid progenitor cells are generated from stem cells and can give rise to a number of cell types such as monocytes, macrophages, neutrophils, mast cells, eosinophils, osteoclasts, microglia and dendritic cells. Upon entry into tumors, myeloid cells can migrate to oxygenated and/or hypoxic areas. Mast cells (MC), eosinophils and tumor-associated macrophages (TAM) accumulating in hypoxic sites can secrete proangiogenic factors. Such myeloid-derived cells can also secrete enzymes that degrade the extracellular matrix (ECM) and release factors such as vascular endothelial growth factor (VEGF), which then promotes angiogenesis. Some types of mast cells and TIE2-expressing monocytes (TEM) that are found near the tumor endothelium, can also release angiogenic factors. Myeloid-derived suppressor cells (MDSCs) and DC are also capable of transdifferentiating into endothelial-like cells in vitro and may become incorporated into new blood vessels in tumors.

However, as described herein, the establishment and growth of metastatic tumor sites can effectively be curtailed by inhibiting expression of versican within myeloid progenitor cells. Inhibition of such myeloid progenitor cells can occur when the myeloid progenitor cells are in the bone marrow or when the myeloid progenitor cells have dispersed from the bone marrow. For example, as illustrated herein, expression of inhibitory short hairpin RNAs that specifically target versican within bone marrow cells effectively inhibits metastasis of breast cancer cells.

Moreover, versican expression is a marker for metastasis. Hence, when heightened versican expression is detected (e.g. compared to control levels of versican expression) treatment of metastatic cancer can be initiated. Such treatment can include administration of chemotherapeutic agents, versican inhibitors, or combinations thereof.

### Versican

Versican is a member of the large chondroitin sulfate proteoglycan (CSPG) family. Versican as described herein is typically mammalian versican, including but not limited to versican from a human, cat, dog, monkey, mouse, rat, or other animal. Versican as described herein may be of any isoform, including VO, V1, and V3.

Sequences are available for various versican proteins and nucleic acids, for example, in the sequence database maintained by the National Center for Biotechnology Information (see website at ncbi.nlm.nih.gov/). One example of a human versican amino acid sequence is available as accession number P13611.3 (GI:2506816), provided below as SEQ ID NO: 1.

| | |
|---|---|
| 1 | MFINIKSILW MCSTLIVTHA LHKVKVGKSP PVRGSLSGKV |
| 61 | SLPCHFSTMP TLPPSYNTSE FLRIKWSKIE VDKNGKDLKE |
| 81 | TTVLVAQNGN IKIGQDYKGR VSVPTHPEAV GDASLTVVKL |
| 121 | LASDAGLYRC DVMYGIEDTQ DTVSLTVDGV VFHYRAATSR |
| 161 | YTLNFEAAQK ACLDVGAVIA TPEQLFAAYE DGFEQCDAGW |
| 201 | LADQTVRYPI RAPRVGCYGD KMGKAGVRTY GFRSPQETYD |
| 241 | VYCYVDHLDG DVFHLTVPSK FTFEEAAKEC ENQDARLATV |
| 281 | GELQAAWRNG FDQCDYGWLS DASVRHPVTV ARAQCGGGLL |
| 301 | GVRTLYRFEN QTGFPPPDSR FDAYCFKPKE ATTIDLSILA |
| 361 | ETASPSLSKE PQMVSDRTTP IIPLVDELPV IPTEFPPVGN |
| 401 | IVSFEQKATV QPQAITDSLA TKLPTPTGST KKPWDMDDYS |
| 441 | PSASGPLGKL DISEIKEEVL QSTTGVSHYA TDSWDGVVED |
| 481 | KQTQESVTQI EQIEVGPLVT SMEILKHIPS KEFPVTETPL |
| 521 | VTARMILESK TEKKMVSTVS ELVTTGHYGF TLGEEDDEDR |
| 561 | TLTVGSDEST LIFDQIPEVI TVSKTSEDTI HTHLEDLESV |
| 601 | SASTTVSPLI MPDNNGSSMD DWEERQTSGR ITEEFLGKYL |
| 641 | STTPFPSQHR TEIELFPYSG DKILVEGIST VIYPSLQTEM |
| 681 | THRRERTETL IPEMRTDTYT DEIQEEITKS PFMGKTEEEV |
| 721 | FSGMKLSTSL SEPIHVTESS VEMTKSFDFP TLITKLSAEP |
| 761 | TEVRDMEEDF TATPGTTKYD ENITTVLLAH GTLSVEAATV |
| 801 | SKWSWDEDNT TSKPLESTEP SASSKLPPAL LTTVGMNGKD |
| 841 | KDIPSFTEDG ADEFTLIPDS TQKQLEEVTD EDIAAHGKFT |
| 881 | IRFQPTTSTG IAEKSTLRDS TTEEKVPPIT STEGQVYATM |
| 921 | EGSALGEVED VDLSKPVSTV PQFAHTSEVE GLAFVSYSST |
| 961 | QEPTTYVDSS HTIPLSVIPK TDWGVLVPSV PSEDEVLGEP |
| 1001 | SQDILVIDQT RLEATISPET MRTTKITEGT TQEEFPWKEQ |
| 1041 | TAEKPVPALS STAWTPKEAV TPLDEQEGDG SAYTVSEDEL |
| 1081 | LTGSERVPVL ETTPVGKIDH SVSYPPGAVT EHKVKTDEVV |
| 1121 | TLTPRIGPKV SLSPGPEQKY ETEGSSTTGF TSSLSPFSTH |
| 1161 | ITQLMEETTT EKTSLEDIDL GSGLFEKPKA TELIEFSTIK |
| 1201 | VTVPSDITTA FSSVDRLHTT SAFKPSSAIT KKPPLIDREP |
| 1241 | GEETTSDMVI IGESTSHVPP TTLEDIVAKE TETDIDREYF |
| 1281 | TTSSPPATQP TRPPTVEDKE AFGPQALSTP QPPASTKFHP |
| 1321 | DINVYIIEVR ENKTGRMSDL SVIGHPIDSE SKEDEPCSEE |
| 1361 | TDPVHDLMAE ILPEFPDIIE IDLYHSEENE EEEEECANAT |
| 1401 | DVTTTPSVQY INGKHLVTTV PKDPEAAEAR RGQFESVAPS |
| 1441 | QNFSDSSESD THPFVIAKTE LSTAVQPNES TETTESLEVT |
| 1481 | WKPETYPETS EHFSGGEPDV FPTVPFHEEF ESGTAKKGAE |
| 1521 | SVTERDTEVG HQAHEHTEPV SLFPEESSGE IAIDQESQKI |
| 1561 | AFARATEVTF GEEVEKSTSV TYTPTIVPSS ASAYVSEEEA |
| 1601 | VTLIGNPWPD DLLSTKESWV EATPRQVVEL SGSSSIPITE |
| 1641 | GSGEAEEDED TMFTMVTDLS QRNTTDTLIT LDTSRIITES |
| 1681 | FFEVPATTIY PVSEQPSAKV VPTKFVSETD TSEWISSTTV |
| 1721 | EEKKRKEEEG TTGTASTFEV YSSTQRSDQL ILPFELESPN |
| 1761 | VATSSDSGTR KSFMSLTTPT QSEREMTDST PVFTETKTLE |
| 1801 | NLGAQTTEHS SIHQPGVQEG LTTLPRSPAS VFMEQGSGEA |
| 1841 | AADPETTTVS SFSLKVEYAI QAEKEVAGTL SPHVETTFST |
| 1881 | EPTGLVLSTV MDRVVAENIT QTSREIVISE RLGEPNYGAE |
| 1921 | IRGFSTGFPL EEDFSGDFRE YSTVSHPIAK EETVMMEGSG |
| 1961 | DAAFRDTQTS PSTVPTSVHI SHISDSEGPS STMVSTSAFP |
| 2001 | WEEFTSSAEG SGEQLVTVSS SVVPVLPSAV QKFSGTASSI |
| 2041 | IDEGLGEVGT VNEIDRRSTI LPTAEVEGTK APVEKEEVKV |
| 2081 | SGTVSTNFPQ TIEPAKLWSR QEVNPVRQEI ESETTSEEQI |
| 2121 | QEEKSFESPQ NSPATEQTIF DSQTFTETEL KTTDYSVLTT |
| 2161 | KKTYSDDKEM KEEDTSLVNM STPDPDANGL ESYTTLPEAT |
| 2201 | EKSHFFLATA LVTESIPAEH VVTDSPIKKE ESTKHFPKGM |
| 2241 | RPTIQESDTE LLFSGLGSGE EVLPTLPTES VNFTEVEQIN |
| 2281 | NTLYPHTSQV ESTSSDKIED FNRMENVAKE VGPLVSQTDI |
| 2321 | FEGSGSVTST TLIEILSDTG AEGPTVAPLP FSTDIGHPQN |
| 2361 | QTVRWAEEIQ TSRPQTITEQ DSNKNSSTAE INETTTSSTD |
| 2401 | FLARAYGFEM AKEFVTSAPK PSDLYYEPSG EGSGEVDIVD |
| 2441 | SFHTSATTQA TRQESSTTFV SDGSLEKHPE VPSAKAVTAD |
| 2481 | GFPTVSVMLP LHSEQNKSSP DPTSTLSNTV SYERSTDGSF |
| 2521 | QDRFREFEDS TLKPNRKKPT ENIIIDLDKE DKDLILTITE |
| 2561 | STILEILPEL TSDKNTIIDI DHTKPVYEDI LGMQTDIDTE |
| 2601 | VPSEPHDSND ESNDDSTQVQ EIYEAAVKLS LTEETFEGSA |
| 2641 | DVLASYTQAT HDESMTYEDR SQLDHMGFHF TTGIPAPSTE |
| 2681 | TELDVLLPTA TSLPIPRKSA TVIPEIEGIK AEAKALDDMF |
| 2721 | ESSTLSDGQA IADQSEIIPT LGQFERTQEE YEDKKHAGPS |
| 2761 | FQPEFSSGAE EALVDHTPYL SIATTHLMDQ SVTEVPDVME |
| 2801 | GSNPPYYTDT TLAVSTFAKL SSQTPSSPLT IYSGSEASGH |
| 2841 | TEIPQPSALP GIDVGSSVMS PQDSFKEIHV NIEATFKPSS |
| 2881 | EEYLHITEPP SLSPDTKLEP SEDDGKPELL EEMEASPTEL |
| 2921 | IAVEGTEILQ DFQNKTDGQV SGEAIKMFPT IKTPEAGTVI |
| 2961 | TTADEIELEG ATQWPHSTSA SATYGVEAGV VPWLSPQTSE |
| 3001 | RPTLSSSPEI NPETQAALIR GQDSTIAASE QQVAARILDS |
| 3041 | NDQATVNPVE FNTEVATPPF SLLETSNETD FLIGINEESV |
| 3081 | EGTAIYLPGP DRCKMNPCLN GGTCYPTETS YVCTCVPGYS |
| 3121 | GDQCELDFDE CHSNPCRNGA TCVDGFNTFR CLCLPSYVGA |
| 3161 | LCEQDTETCD YGWHKFQGQC YKYFAHRRTW DAAERECRLQ |
| 3201 | GAHLTSILSH EEQMFVNRVG HDYQWIGLND KMFEHDFRWT |
| 3241 | DGSTLQYENW RPNQPDSFFS AGEDCVVIIW HENGQWNDVP |
| 3281 | CNYHLTYTCK KGTVACGQPP VVENAKTFGK MKPRYEINSL |
| 3321 | IRYHCKDGFI QRHLPTIRCL GNGRWAIPKI TCMNPSAYQR |
| 3361 | TYSMKYFKNS SSAKDNSINT SKHDHRWSRR WQESRR |

Another example of a versican protein sequence is the versican core protein isoform 1 precursor [Homo sapiens] available as NCBI accession number NP_{—}004376.2 (GI:21361116), and provided below as SEQ ID NO:2.

| | |
|---|---|
| 1 | MFINIKSILW MCSTLIVTHA LHKVKVGKSP PVRGSLSGKV |
| 41 | SLPCHFSTMP TLPPSYNTSE FLRIKWSKTE VDKNGKDLKE |
| 81 | TTVLVAQMGN IKIGQDYKGR VSVPTHPEAV GDASLTVVKL |
| 121 | LASDAGLYRC DVMYGIEDTQ DTVSLTVDGV VFHYRAATSR |
| 161 | YTLKFEAAQK ACLDVGAVIA TPEQLFAAYE DGFEQCDAGW |
| 201 | LADQTVRYPI RAPRVGCYGD KMGKAGVRTY GFRSPQETYD |
| 241 | VYCYVDHLDG DVFHLTVPSK FTFEEAAKEC ENQDARLATV |
| 281 | GELQAAWRNG FDQCDYGWLS DASVRHPVTV ARAQCGGGLL |
| 321 | GVRTLYRFEK QTGFPPPDSR FDAYCFKPKE ATTIDLSILA |
| 361 | ETASPSLSKE PQMVSDRTTP IIPLVDELPV IPTEFPPVGN |
| 401 | IVSFEQKATV QPQAITDSLA TKLPTPTGST KKPWDMDDYS |
| 441 | PSASGPLGKL DISEIKEEVL QSTTGVSHYA TDSWDGVVED |
| 481 | KQTQESVTQI EQIEVGPLVT SMEILKHIPS KEFPVTETPL |
| 521 | VTARMILESK TEKKMVSTVS ELVTTGHYGF TLGEEDDEDR |
| 561 | TLTVGSDEST LIFDQIPEVI TVSKTSEDTI HTHLEDLESV |
| 601 | SASTTVSPLI MPDNKGSSMD DWEERQTSGR ITEEFLGKYL |
| 641 | STTPFPSQHR TEIELFPYSG DKILVEGIST VIYPSLQTEM |
| 681 | THRRERTETL IPEMRTDTYT DEIQEEITKS PFMGKTEEEV |
| 721 | FSGMKLSTSL SEPIHVTESS VEMTKSFDFP TLITKLSAEP |
| 761 | TEVRDMEEDF TATPGTTKYD ENITTVLLAH GTLSVEAATV |
| 801 | SKWSWDEDNT TSKPLESTEP SASSKLPPAL LTTVGMNGKD |
| 841 | KDIPSFTEDG ADEFTLIPDS TQKQLEEVTD EDIAAHGKFT |
| 881 | IRFQPTTSTG IAEKSTLRDS TTEEKVPPIT STEGQVYATM |
| 921 | EGSALGEVED VDLSKPVSTV PQFAHTSEVE GLAFVSYSST |
| 961 | QEPTTYVDSS HTIPLSVIPK TDWGVLVPSV PSEDEVLGEP |
| 1001 | SQDILVIDQT RLEATISPET MRTTKITEGT TQEEFPWKEQ |
| 1041 | TAEKPVPALS STAWTPKEAV TPLDEQEGDG SAYTVSEDEL |
| 1081 | LTGSERVPVL ETTPVGKIDH SVSYPPGAVT EHKVKTDEVV |
| 1121 | TLTPRIGPKV SLSPGPEQKY ETEGSSTTGF TSSLSPFSTH |
| 1161 | ITQLMEETTT EKTSLEDIDL GSGLFEKPKA TELIEFSTIK |
| 1201 | VTVPSDITTA FSSVDRLHTT SAFKPSSAIT KKPPLIDREP |
| 1241 | GEETTSDMVI IGESTSHVPP TTLEDIVAKE TETDIDREYF |
| 1281 | TTSSPPATQP TRPPTVEDKE AFGPQALSTP QPPASTKFHP |
| 1321 | DINVYIIEVR ENKTGRMSDL SVIGHPIDSE SKEDEPCSEE |
| 1361 | TDPVHDLMAE ILPEFPDIIE IDLYHSEENE EEEEECANAT |
| 1401 | DVTTTPSVQY INGKHLVTTV PKDPEAAEAR RGQFESVAPS |
| 1441 | QNFSDSSESD THPFVIAKTE LSTAVQPNES TETTESLEVT |
| 1481 | WKPETYPETS EHFSGGEPDV FPTVPFHEEF ESGTAKKGAE |
| 1521 | SVTERDTEVG HQAHEHTEPV SLFPEESSGE IAIDQESQKI |
| 1561 | AFARATEVTF GEEVEKSTSV TYTPTIVPSS ASAYVSEEEA |
| 1601 | VTLIGNPWPD DLLSTKESWV EATPRQVVEL SGSSSIPITE |
| 1641 | GSGEAEEDED TMFTMVTDLS QRNTTDTLIT LDTSRIITES |
| 1681 | FFEVPATTIY PVSEQPSAKV VPTKFVSETD TSEWISSTTV |
| 1721 | EEKKRKEEEG TTGTASTFEV YSSTQRSDQL ILPFELESPN |
| 1761 | VATSSDSGTR KSFMSLTTPT QSEREMTDST PVFTETNTLE |
| 1801 | NLGAQTTEHS SIHQPGVQEG LTTLPRSPAS VFMEQGSGEA |
| 1841 | AADPETTTVS SFSLNVEYAI QAEKEVAGTL SPHVETTFST |
| 1881 | EPTGLVLSTV MDRVVAENIT QTSREIVISE RLGEPNYGAE |
| 1921 | IRGFSTGFPL EEDFSGDFRE YSTVSHPIAK EETVMMEGSG |
| 1961 | DAAFRDTQTS PSTVPTSVHI SHISDSEGPS STMVSTSAFP |
| 2001 | WEEFTSSAEG SGEQLVTVSS SVVPVLPSAV QKFSGTASSI |
| 2041 | IDEGLGEVGT VNEIDRRSTI LPTAEVEGTK APVEKEEVKV |
| 2081 | SGTVSTNFPQ TIEPAKLWSR QEVNPVRQEI ESETTSEEQI |
| 2121 | QEEKSFESPQ NSPATEQTIF DSQTFTETEL KTTDYSVLTT |
| 2161 | KKTYSDDKEM KEEDTSLVNM STPDPDANGL ESYTTLPEAT |
| 2201 | EKSHFFLATA LVTESIPAEH VVTDSPIKKE ESTKHFPKGM |
| 2241 | RPTIQESDTE LLFSGLGSGE EVLPTLPTES VNFTEVEQIN |
| 2281 | NTLYPHTSQV ESTSSDKIED FNRMENVAKE VGPLVSQTDI |
| 2321 | FEGSGSVTST TLIEILSDTG AEGPTVAPLP FSTDIGHPQN |
| 2361 | QTVRWAEEIQ TSRPQTITEQ DSNKNSSTAE INETTTSSTD |
| 2401 | FLARAYGFEM AKEFVTSAPK PSDLYYEPSG EGSGEVDIVD |
| 2441 | SFHTSATTQA TRQESSTTFV SDGSLEKHPE VPSAKAVTAD |
| 2481 | GFPTVSVMLP LHSEQNKSSP DPTSTLSNTV SYERSTDGSF |
| 2521 | QDRFREFEDS TLKPNRKKPT ENIIIDLDKE DKDLILTITE |
| 2561 | STILEILPEL TSDKNTIIDI DHTKPVYEDI LGMQTDIDTE |
| 2601 | VPSEPHDSND ESNDDSTQVQ EIYEAAVNLS LTEETFEGSA |
| 2641 | DVLASYTQAT HDESMTYEDR SQLDHMGFHF TTGIPAPSTE |
| 2681 | TELDVLLPTA TSLPIPRKSA TVIPEIEGIK AEAKALDDMF |
| 2721 | ESSTLSDGQA IADQSEIIPT LGQFERTQEE YEDKKHAGPS |
| 2761 | FQPEFSSGAE EALVDHTPYL SIATTHLMDQ SVTEVPDVME |
| 2801 | GSNPPYYTDT TLAVSTFAKL SSQTPSSPLT IYSGSEASGH |
| 2841 | TEIPQPSALP GIDVGSSVMS PQDSFKEIHV NIEATFKPSS |
| 2881 | EEYLHITEPP SLSPDTKLEP SEDDGKPELL EEMEASPTEL |
| 2921 | IAVEGTEILQ DFQNKTDGQV SGEAIKMFPT IKTPEAGTVI |
| 2961 | TTADEIELEG ATQWPHSTSA SATYGVEAGV VPWLSPQTSE |
| 3001 | RPTLSSSPEI NPETQAALIR GQDSTIAASE QQVAARILDS |
| 3041 | NDQATVNPVE FNTEVATPPF SLLETSNETD FLIGINEESV |
| 3081 | EGTAIYLPGP DRCKMNPCLN GGTCYPTETS YVCTCVPGYS |
| 3121 | GDQCELDFDE CHSNPCRNGA TCVDGFNTFR CLCLPSYVGA |
| 3161 | LCEQDTETCD YGWHKFQGQC YKYFAHRRTW DAAERECRLQ |
| 3201 | GAHLTSILSH EEQMFVNRVG HDYQWIGLND KMFEHDFRWT |
| 3241 | DGSTLQYENW RPNQPDSFFS AGEDCVVIIW HENGQWNDVP |
| 3281 | CNYHLTYTCK KGTVACGQPP VVENAKTFGK MKPRYEINSL |
| 3321 | IRYHCKDGFI QRHLPTIRCL GNGRWAIPKI TCMNPSAYQR |
| 3361 | TYSMKYFKNS SSAKDNSINT SKHDHRWSRR WQESRR |

A nucleotide sequence for the SEQ ID NO:2 protein is available as NCBI accession number NM_{—}004385.4 GI:255918074. shown below as SEQ ID NO:3.

| | |
|---|---|
| 1 | CTTCTTCTCG CTGAGTCTCC TCCTCGGCTC TGACGGTACA |
| 41 | GTGATATAAT GATGATGGGT GTCACAACCC GCATTTGAAC |
| 81 | TTGCAGGCGA GCTGCCCCGA GCCTTTCTGG GGAAGAACTC |
| 121 | CAGGCGTGCG GACGCAACAG CCGAGAACAT TAGGTGTTGT |
| 161 | GGACAGGAGC TGGGACCAAG ATCTTCGGCC AGCCCCGCAT |
| 201 | CCTCCCGCAT CTTCCAGCAC CGTCCCGCAC CCTCCGCATC |
| 241 | CTTCCCCGGG CCACCACGCT TCCTATGTGA CCCGCCTGGG |
| 281 | CAACGCCGAA CCCAGTCGCG CAGCGCTGCA GTGAATTTTC |
| 321 | CCCCCAAACT GCAATAAGCC GCCTTCCAAG GCCAAGATGT |
| 361 | TCATAAATAT AAAGAGCATC TTATGGATGT GTTCAACCTT |
| 401 | AATAGTAACC CATGCGCTAC ATAAAGTCAA AGTGGGAAAA |
| 441 | AGCCCACCGG TGAGGGGCTC CCTCTCTGGA AAAGTCAGCC |
| 481 | TACCTTGTCA TTTTTCAACG ATGCCTACTT TGCCACCCAG |
| 521 | TTACAACACC AGTGAATTTC TCCGCATCAA ATGGTCTAAG |
| 561 | ATTGAAGTGG ACAAAAATGG AAAAGATTTG AAAGAGACTA |
| 601 | CTGTCCTTGT GGCCCAAAAT GGAAATATCA AGATTGGTCA |
| 641 | GGACTAGAAA GGGAGAGTGT CTGTGCCCAC ACATCCCGAG |
| 681 | GCTGTGGGCG ATGCCTCCCT CACTGTGGTC AAGCTGCTGG |
| 721 | CAAGTGATGC GGGTCTTTAC CGCTGTGACG TCATGTACGG |
| 761 | GATTGAAGAC ACACAAGACA CGGTGTCACT GACTGTGGAT |
| 801 | GGGGTTGTGT TTCACTACAG GGCGGCAACC AGCAGGTACA |
| 841 | CACTGAATTT TGAGGCTGCT CAGAAGGCTT GTTTGGACGT |
| 881 | TGGGGCAGTC ATAGCAACTC CAGAGCAGCT CTTTGCTGCC |
| 921 | TATGAAGATG GATTTGAGCA GTGTGACGCA GGCTGGCTGG |
| 961 | CTGATCAGAC TGTCAGATAT CCCATCCGGG CTCCCAGAGT |
| 1001 | AGGCTGTTAT GGAGATAAGA TGGGAAAGGC AGGAGTCAGG |
| 1041 | ACTTATGGAT TCCGTTCTCC CCAGGAAACT TACGATGTGT |
| 1081 | ATTGTTATGT GGATCATCTG GATGGTGATG TGTTCCACCT |
| 1121 | CACTGTCCCC AGTAAATTCA CCTTCGAGGA GGCTGCAAAA |
| 1161 | GAGTGTGAAA ACCAGGATGC CAGGCTGGCA ACAGTGGGGG |
| 1201 | AACTCCAGGC GGCATGGAGG AACGGCTTTG ACCAGTGCGA |
| 1241 | TTACGGGTGG CTGTCGGATG CCAGCGTGCG CCACCCTGTG |
| 1281 | ACTGTGGCCA GGGCCCAGTG TGGAGGTGGT CTACTTGGGG |
| 1321 | TGAGAACCCT GTATCGTTTT GAGAACCAGA CAGGCTTCCC |
| 1361 | TCCCCCTGAT AGCAGATTTG ATGCCTACTG CTTTAAACCT |
| 1401 | AAAGAGGCTA CAACCATCGA TTTGAGTATC CTCGCAGAAA |
| 1441 | CTGCATCACC CAGTTTATCC AAAGAACCAC AAATGGTTTC |
| 1481 | TGATAGAACT ACACCAATCA TCCCTTTAGT TGATGAATTA |
| 1521 | CCTGTCATTC CAACAGAGTT CCCTCCCGTG GGAAATATTG |
| 1561 | TCAGTTTTGA ACAGAAAGCC ACAGTCCAAC CTCAGGCTAT |
| 1601 | CACAGATAGT TTAGCCACCA AATTACCCAC ACCTACTGGC |
| 1641 | AGTACCAAGA AGCCCTGGGA TATGGATGAC TACTCACCTT |
| 1681 | CTGCTTCAGG ACCTCTTGGA AAGCTAGACA TATCAGAAAT |
| 1721 | TAAGGAAGAA GTGCTCCAGA GTACAACTGG CGTCTCTCAT |
| 1761 | TATGCTACGG ATTCATGGGA TGGTGTCGTG GAAGATAAAC |
| 1801 | AAACACAAGA ATCGGTTACA CAGATTGAAC AAATAGAAGT |
| 1841 | GGGTCCTTTG GTAACATCTA TGGAAATCTT AAAGCACATT |
| 1881 | CCTTCCAAGG AATTCCCTGT AACTGAAACA CCATTGGTAA |
| 1921 | CTGCAAGAAT GATCCTGGAA TCCAAAACTG AAAAGAAAAT |
| 1961 | GGTAAGCACT GTTTCTGAAT TGGTAACCAC AGGTCACTAT |
| 2001 | GGATTCACCT TGGGAGAAGA GGATGATGAA GACAGAACAC |
| 2041 | TTACAGTTGG ATCTGATGAG AGCACCTTGA TCTTTGACCA |
| 2081 | AATTCCTGAA GTCATTACGG TGTCAAAGAC TTCAGAAGAC |
| 2121 | ACCATCCACA CTCATTTAGA AGACTTGGAG TCAGTCTCAG |
| 2161 | CATCCACAAC TGTTTCCCCT TTAATTATGC CTGATAATAA |
| 2201 | TGGATCATCC ATGGATGACT GGGAAGAGAG ACAAACTAGT |
| 2241 | GGTAGGATAA CGGAAGAGTT TCTTGGCAAA TATCTGTCTA |
| 2281 | CTACACCTTT TCCATCACAG CATCGTACAG AAATAGAATT |
| 2321 | GTTTCCTTAT TCTGGTGATA AAATATTAGT AGAGGGAATT |
| 2361 | TCCACAGTTA TTTATCCTTC TCTAGAAACA GAAATGACAC |
| 2401 | ATAGAAGAGA AAGAACAGAA ACACTAATAC CAGAGATGAG |
| 2441 | AACAGATACT TATACAGATG AAATACAAGA AGAGATCACT |
| 2481 | AAAAGTCCAT TTATGGGAAA AACAGAAGAA GAAGTCTTCT |
| 2521 | CTGGGATGAA ACTCTCTACA TCTCTCTCAG AGCCAATTCA |
| 2561 | TGTTACAGAG TCTTCTGTGG AAATGACCAA GTCTTTTGAT |
| 2601 | TTCCCAACAT TGATAACAAA GTTAAGTGCA GAGCCAACAG |
| 2641 | AAGTAAGAGA TATGGAGGAA GACTTTACAG CAACTCCAGG |
| 2681 | TACTACAAAA TATGATGAAA ATATTACAAC AGTGCTTTTG |
| 2721 | GCCCATGGTA CTTTAAGTGT TGAAGCAGCC ACTGTATCAA |
| 2761 | AATGGTCATG GGATGAAGAT AATACAACAT CCAAGCCTTT |
| 2801 | AGAGTCTACA GAACCTTCAG CCTCTTCAAA ATTGCCCCCT |
| 2841 | GCCTTACTCA CAACTGTGGG GATGAATGGA AAGGATAAAG |
| 2881 | ACATCCCAAG TTTCACTGAA GATGGAGCAG ATGAATTTAC |
| 2921 | TCTTATTCCA GATAGTACTC AAAAGCAGTT AGAGGAGGTT |
| 2961 | ACTGATGAAG ACATAGCAGC CCATGGAAAA TTCACAATTA |
| 3001 | GATTTCAGCC AACTACATCA ACTGGTATTG CAGAAAAGTC |
| 3041 | AACTTTGAGA GATTCTACAA CTGAAGAAAA AGTTCCACCT |
| 3081 | ATCACAAGCA CTGAAGGCCA AGTTTATGCA ACCATGGAAG |
| 3121 | GAAGTGCTTT GGGTGAAGTA GAAGATGTGG ACCTCTCTAA |
| 3161 | GCCAGTATCT ACTGTTCCCC AATTTGCACA CACTTCAGAG |
| 3201 | GTGGAAGGAT TAGCATTTGT TAGTTATAGT AGCACCCAAG |
| 3241 | AGCCTACTAC TTATGTAGAC TCTTCCCATA CCATTCCTCT |
| 3281 | TTCTGTAATT CCCAAGACAG ACTGGGGAGT GTTAGTACCT |
| 3321 | TCTGTTCCAT CAGAAGATGA AGTTCTAGGT GAACCCTCTC |
| 3361 | AAGACATACT TGTCATTGAT CAGACTCGCC TTGAAGCGAC |
| 3401 | TATTTCTCCA GAAACTATGA GAACAACAAA AATCACAGAG |
| 3441 | GGAACAACTC AGGAAGAATT CCCTTGGAAA GAACAGACTG |
| 3481 | CAGAGAAACC AGTTCCTGCT CTCAGTTCTA CAGCTTGGAC |
| 3521 | TCCCAAGGAG GCAGTAACAC CACTGGATGA ACAAGAGGGC |
| 3561 | GATGGATCAG CATATACAGT CTCTGAAGAT GAATTGTTGA |
| 3601 | CAGGTTCTGA GAGGGTCCCA GTTTTAGAAA CAACTCCAGT |
| 3641 | TGGAAAAATT GATCACAGTG TGTCTTATCC ACCAGGTGCT |
| 3681 | GTAACTGAGC ACAAAGTGAA AACAGATGAA GTGGTAACAC |
| 3721 | TAACACCACG CATTGGGCCA AAAGTATCTT TAAGTCCAGG |
| 3761 | GCCTGAACAA AAATATGAAA CAGAAGGTAG TAGTACAACA |
| 3801 | GGATTTACAT CATCTTTGAG TCCTTTTAGT ACCCACATTA |
| 3841 | CCCAGCTTAT GGAAGAAACC ACTACTGAGA AAACATCCCT |
| 3881 | AGAGGATATT GATTTAGGCT CAGGATTATT TGAAAAGCCC |
| 3921 | AAAGCCACAG AACTCATAGA ATTTTCAACA ATCAAAGTCA |
| 3961 | CAGTTCCAAG TGATATTACC ACTGCCTTCA GTTCAGTAGA |
| 4001 | CAGACTTCAC ACAACTTCAG CATTCAAGCC ATCTTCCGCG |
| 4041 | ATCACTAAGA AACCACCTCT CATCGACAGG GAACCTGGTG |
| 4081 | AAGAAACAAC CAGTGACATG GTAATCATTG GAGAATCAAC |
| 4121 | ATCTCATGTT CCTCCCACTA CCCTTGAAGA TATTGTAGCC |
| 4161 | AAGGAAACAG AAACCGATAT TGATAGAGAG TATTTCACGA |
| 4201 | CTTCAAGTCC TCCTGCTACA CAGCCAACAA GACCACCCAC |
| 4241 | TGTGGAAGAC AAAGAGGCCT TTGGACCTCA GGCGCTTTCT |
| 4281 | ACGCCACAGC CCCCAGCAAG CACAAAATTT CACCCTGACA |
| 4321 | TTAATGTTTA TATTATTGAG GTCAGAGAAA ATAAGACAGG |
| 4361 | TCGAATGAGT GATTTGAGTG TAATTGGTCA TCCAATAGAT |
| 4401 | TCAGAATCTA AAGAAGATGA ACCTTGTAGT GAAGAAACAG |
| 4441 | ATCCAGTGCA TGATCTAATG GCTGAAATTT TACCTGAATT |
| 4481 | CCCTGACATA ATTGAAATAG ACCTATACCA CAGTGAAGAA |
| 4521 | AATGAAGAAG AAGAAGAAGA GTGTGCAAAT GCTACTGATG |
| 4561 | TGACAACCAC CCCATCTGTG CAGTACATAA ATGGGAAGCA |
| 4601 | TCTCGTTACC ACTGTGCCCA AGGACCCAGA AGCTGCAGAA |
| 4641 | GCTAGGCGTG GCCAGTTTGA AAGTGTTGCA CCTTCTCAGA |
| 4681 | ATTTCTCGGA CAGCTCTGAA AGTGATACTC ATCCATTTGT |
| 4721 | AATAGCCAAA ACGGAATTGT CTACTGCTGT GCAACCTAAT |
| 4761 | GAATCTACAG AAACAACTGA GTCTCTTGAA GTTACATGGA |
| 4801 | AGCCTGAGAC TTACCCTGAA ACATCAGAAC ATTTTTCAGG |
| 4841 | TGGTGAGCCT GATGTTTTCC CCACAGTCCC ATTCCATGAG |
| 4881 | GAATTTGAAA GTGGAACAGC CAAAAAAGGG GCAGAATCAG |
| 4921 | TCACAGAGAG AGATACTGAA GTTGGTCATC AGGCACATGA |
| 4961 | ACATACTGAA CCTGTATCTC TGTTTCCTGA AGAGTCTTCA |
| 5001 | GGAGAGATTG CCATTGACCA AGAATCTCAG AAAATAGCCT |
| 5041 | TTGCAAGGGC TACAGAAGTA ACATTTGGTG AAGAGGTAGA |
| 5081 | AAAAAGTACT TCTGTCACAT ACACTCCCAC TATAGTTCCA |
| 5121 | AGTTCTGCAT CAGCATATGT TTCAGAGGAA GAAGCAGTTA |
| 5161 | CCCTAATAGG AAATCCTTGG CCAGATGACC TGTTGTCTAC |
| 5201 | CAAAGAAAGC TGGGTAGAAG CAACTCCTAG ACAAGTTGTA |
| 5241 | GAGCTCTCAG GGAGTTCTTC GATTCCAATT ACAGAAGGCT |
| 5281 | CTGGAGAAGC AGAAGAAGAT GAAGATACAA TGTTCACCAT |
| 5321 | GGTAACTGAT TTATCACAGA GAAATACTAG TGATACACTC |
| 5361 | ATTACTTTAG ACACTAGCAG GATAATCACA GAAAGCTTTT |
| 5401 | TTGAGGTTCC TGCAACCACC ATTTATCCAG TTTCTGAACA |
| 5441 | ACCTTCTGCA AAAGTGGTGC CTACCAAGTT TGTAAGTGAA |
| 5481 | ACAGACACTT CTGAGTGGAT TTCCAGTACC ACTGTTGAGG |
| 5521 | AAAAGAAAAG GAAGGAGGAG GAGGGAACTA CAGGTACGGC |
| 5561 | TTCTACATTT GAGGTATATT CATCTACACA GAGATCGGAT |
| 5601 | CAATTAATTT TACCCTTTGA ATTAGAAAGT CCAAATGTAG |
| 5641 | CTACATCTAG TGATTCAGGT ACCAGGAAAA GTTTTATGTC |
| 5781 | CTTGACAACA CCAACACAGT CTGAAAGGGA AATGACAGAT |
| 5721 | TCTACTCCTG TCTTTACAGA AACAAATACA TTAGAAAATT |
| 5761 | TGGGGGCACA GACCACTGAG CACAGCAGTA TCCATCAACC |
| 5801 | TGGGGTTCAG GAAGGGCTGA CCACTCTCCC ACGTAGTCCT |
| 5841 | GCCTCTGTCT TTATGGAGCA GGGCTCTGGA GAAGCTGCTG |
| 5881 | CCGACCCAGA AACCACCACT GTTTCTTCAT TTTCATTAAA |
| 5921 | CGTAGAGTAT GCAATTCAAG CCGAAAAGGA AGTAGCTGGC |
| 5961 | ACTTTGTCTC CGCATGTGGA AACTACATTC TCCACTGAGC |
| 6001 | CAACAGGACT GGTTTTGAGT ACAGTAATGG ACAGAGTAGT |
| 6041 | TGCTGAAAAT ATAACCCAAA CATCCAGGGA AATAGTGATT |
| 6081 | TCAGAGCGAT TAGGAGAACC AAATTATGGG GCAGAAATAA |
| 6121 | GGGGCTTTTC CACAGGTTTT CCTTTGGAGG AAGATTTCAG |
| 6161 | TGGTGACTTT AGAGAATACT CAACAGTGTC TCATCCCATA |
| 6201 | GCAAAAGAAG AAACGGTAAT GATGGAAGGC TCTGGAGATG |
| 6241 | CAGCATTTAG GGACACCCAG ACTTCACCAT CTACAGTACC |
| 6281 | TACTTCAGTT CACATCAGTC ACATATCTGA CTCAGAAGGA |
| 6321 | CCCAGTAGCA CCATGGTCAG CACTTCAGCC TTCCCCTGGG |
| 6361 | AAGAGTTTAC ATCCTCAGCT GAGGGCTCAG GTGAGCAACT |
| 6401 | GGTCACAGTC AGCAGCTCTG TTGTTCCAGT GCTTCCCAGT |
| 6441 | GCTGTGCAAA AGTTTTCTGG TACAGCTTCC TCCATTATCG |
| 6481 | ACGAAGGATT GGGAGAAGTG GGTACTGTCA ATGAAATTGA |
| 6521 | TAGAAGATCC ACCATTTTAC CAACAGCAGA AGTGGAAGGT |
| 6561 | ACGAAAGCTC CAGTAGAGAA GGAGGAAGTA AAGGTCAGTG |
| 6601 | GCACAGTTTC AACAAACTTT CCCCAAACTA TAGAGCCAGC |
| 6641 | CAAATTATGG TCTAGGCAAG AAGTCAACCC TGTAAGACAA |
| 6681 | GAAATTGAAA GTGAAACAAC ATCAGAGGAA CAAATTCAAG |
| 6721 | AAGAAAAGTC ATTTGAATCC CCTCAAAACT CTCCTGCAAC |
| 6761 | AGAACAAACA ATCTTTGATT CACAGACATT TACTGAAACT |
| 6801 | GAACTCAAAA CCACAGATTA TTCTGTACTA ACAACAAAGA |
| 6841 | AAACTTACAG TGATGATAAA GAAATGAAGG AGGAAGACAC |
| 6881 | TTCTTTAGTT AACATGTCTA CTCCAGATCC AGATGCAAAT |
| 6921 | GGCTTGGAAT CTTACACAAC TCTCCCTGAA GCTACTGAAA |
| 6961 | AGTCACATTT TTTCTTAGCT ACTGCATTAG TAACTGAATC |
| 7001 | TATACCAGCT GAACATGTAG TCACAGATTC ACCAATCAAA |
| 7041 | AAGGAAGAAA GTACAAAACA TTTTCCGAAA GGCATGAGAC |
| 7081 | CAACAATTCA AGAGTCAGAT ACTGAGCTCT TATTCTCTGG |
| 7121 | ACTGGGATCA GGAGAAGAAG TTTTACCTAC TCTACCAACA |
| 7161 | GAGTCAGTGA ATTTTACTGA AGTGGAACAA ATCAATAACA |
| 7201 | CATTATATCC CCACACTTCT CAAGTGGAAA GTACCTCAAG |
| 7241 | TGACAAAATT GAAGACTTTA ACAGAATGGA AAATGTGGCA |
| 7281 | AAAGAAGTTG GACCACTCGT ATCTCAAACA GACATCTTTG |
| 7321 | AAGGTAGTGG GTCAGTAACC AGCACAACAT TAATAGAAAT |
| 7361 | TTTAAGTGAC ACTGGAGCAG AAGGACCCAC GGTGGCACCT |
| 7401 | CTCCCTTTCT CCACGGACAT CGGACATCCT CAAAATCAGA |
| 7441 | CTGTCAGGTG GGCAGAAGAA ATCCAGACTA GTAGACCACA |
| 7481 | AACCATAACT GAACAAGACT CTAACAAGAA TTCTTCAACA |
| 7521 | GCAGAAATTA ACGAAACAAC AACCTCATCT ACTGATTTTC |
| 7561 | TGGCTAGAGC TTATGGTTTT GAAATGGCCA AAGAATTTGT |
| 7601 | TACATCAGCA CCAAAACCAT CTGACTTGTA TTATGAACCT |
| 7641 | TCTGGAGAAG GATCTGGAGA AGTGGATATT GTTGATTCAT |
| 7681 | TTCACACTTC TGCAACTACT CAGGCAACCA GACAAGAAAG |
| 7721 | CAGCACCACA TTTGTTTCTG ATGGGTCCCT GGAAAAACAT |
| 7761 | CCTGAGGTGC CAAGCGCTAA AGCTGTTACT GCTGATGGAT |
| 7801 | TCCCAACAGT TTCAGTGATG CTGCCTCTTC ATTCAGAGCA |
| 7841 | GAACAAAAGC TCCCCTGATC CAACTAGCAC ACTGTCAAAT |
| 7881 | ACAGTGTCAT ATGAGAGGTC CACAGACGGT AGTTTCCAAG |
| 7921 | ACCGTTTCAG GGAATTCGAG GATTCCACCT TAAAACCTAA |
| 7961 | CAGAAAAAAA CCCACTGAAA ATATTATCAT AGACCTGGAC |
| 8001 | AAAGAGGACA AGGATTTAAT ATTGACAATT ACAGAGAGTA |
| 8041 | CCATCCTTGA AATTCTACCT GAGCTGACAT CGGATAAAAA |
| 8081 | TACTATCATA GATATTGATC ATACTAAACC TGTGTATGAA |
| 8121 | GACATTCTTG GAATGCAAAC AGATATAGAT ACAGAGGTAC |
| 8161 | CATCAGAACC ACATGACAGT AATGATGAAA GTAATGATGA |
| 8201 | CAGCACTCAA GTTCAAGAGA TCTATGAGGC AGCTGTCAAC |
| 8241 | CTTTCTTTAA CTGAGGAAAC ATTTGAGGGC TCTGCTGATG |
| 8281 | TTCTGGCTAG CTAGACTCAG GCAACACATG ATGAATCAAT |
| 8321 | GACTTATGAA GATAGAAGCC AACTAGATCA CATGGGCTTT |
| 8361 | CACTTCACAA CTGGGATCCC TGCTCCTAGC ACAGAAACAG |
| 8401 | AATTAGACGT TTTACTTCCC ACGGCAACAT CCCTGCCAAT |
| 8441 | TCCTCGTAAG TCTGCCACAG TTATTCCAGA GATTGAAGGA |
| 8481 | ATAAAAGCTG AAGCAAAAGC CCTGGATGAC ATGTTTGAAT |
| 8521 | CAAGCACTTT GTCTGATGGT CAAGCTATTG CAGACCAAAG |
| 8561 | TGAAATAATA CCAACATTGG GCCAATTTGA AAGGACTCAG |
| 8601 | GAGGAGTATG AAGACAAAAA ACATGCTGGT CCTTCTTTTC |
| 8641 | AGCCAGAATT CTCTTCAGGA GCTGAGGAGG CATTAGTAGA |
| 8681 | CCATACTCCC TATCTAAGTA TTGCTACTAC CCACCTTATG |
| 8721 | GATCAGAGTG TAACAGAGGT GCCTGATGTG ATGGAAGGAT |
| 8761 | CCAATCCCCC ATATTACACT GATACAACAT TAGCAGTTTC |
| 8801 | AACATTTGCG AAGTTGTCTT CTCAGACACC ATCATCTCCC |
| 8841 | CTCACTATCT ACTCAGGCAG TGAAGCCTCT GGACACACAG |
| 8881 | AGATCCCCCA GCCCAGTGCT CTGCCAGGAA TAGACGTCGG |
| 8921 | CTCATCTGTA ATGTCCCCAC AGGATTCTTT TAAGGAAATT |
| 8961 | CATGTAAATA TTGAAGCGAC TTTCAAACCA TCAAGTGAGG |
| 9001 | AATACCTTCA CATAACTGAG CCTCCCTCTT TATCTCCTGA |
| 9041 | CACAAAATTA GAACCTTCAG AAGATGATGG TAAACCTGAG |
| 9081 | TTATTAGAAG AAATGGAAGC TTCTCCCACA GAACTTATTG |
| 9121 | CTGTGGAAGG AACTGAGATT CTCCAAGATT TCCAAAACAA |
| 9161 | AACCGATGGT CAAGTTTCTG GAGAAGCAAT CAAGATGTTT |
| 9201 | CCCACCATTA AAACACCTGA GGCTGGAACT GTTATTACAA |
| 9241 | CTGCCGATGA AATTGAATTA GAAGGTGCTA CACAGTGGCC |
| 9281 | ACACTCTACT TCTGCTTCTG CCACCTATGG GGTCGAGGCA |
| 9321 | GGTGTGGTGC CTTGGCTAAG TCCACAGACT TCTGAGAGGC |
| 9361 | CCACGCTTTC TTCTTCTCCA GAAATAAACC CTGAAACTCA |
| 9401 | AGCAGCTTTA ATCAGAGGGC AGGATTCCAC GATAGCAGCA |
| 9441 | TCAGAACAGC AAGTGGCAGC GAGAATTCTT GATTCCAATG |
| 9481 | ATCAGGCAAC AGTAAACCCT GTGGAATTTA ATACTGAGGT |
| 9521 | TGCAACACCA CCATTTTCCC TTCTGGAGAC TTCTAATGAA |
| 9561 | ACAGATTTCC TGATTGGCAT TAATGAAGAG TCAGTGGAAG |
| 9601 | GCACGGCAAT CTATTTACCA GGACCTGATC GCTGCAAAAT |
| 9641 | GAACCCGTGC CTTAACGGAG GCACCTGTTA TCCTACTGAA |
| 9681 | ACTTCCTACG TATGCACCTG TGTGCCAGGA TACAGCGGAG |
| 9721 | ACCAGTGTGA ACTTGATTTT GATGAATGTC ACTCTAATCC |
| 9761 | CTGTCGTAAT GGAGCCACTT GTGTTGATGG TTTTAACACA |
| 9801 | TTCAGGTGCC TCTGCCTTCC AAGTTATGTT GGTGCACTTT |
| 9841 | GTGAGCAAGA TACCGAGACA TGTGACTATG GCTGGCACAA |
| 9881 | ATTCCAAGGG CAGTGCTACA AATACTTTGC CCATCGACGC |
| 9921 | ACATGGGATG CAGCTGAACG GGAATGCCGT CTGCAGGGTG |
| 9961 | CCCATCTCAC AAGCATCCTG TCTCACGAAG AACAAATGTT |
| 10001 | TGTTAATCGT GTGGGCCATG ATTATCAGTG GATAGGCCTC |
| 10041 | AATGACAAGA TGTTTGAGCA TGACTTCCGT TGGACTGATG |
| 10081 | GCAGCACACT GCAATACGAG AATTGGAGAC CCAACCAGCC |
| 10121 | AGACAGCTTC TTTTCTGCTG GAGAAGACTG TGTTGTAATC |
| 10161 | ATTTGGCATG AGAATGGCCA GTGGAATGAT GTTCCCTGCA |
| 10201 | ATTACCATCT CACCTATACG TGCAAGAAAG GAACAGTCGC |
| 10241 | TTGCGGCCAG CCCCCTGTTG TAGAAAATGC CAAGACCTTT |
| 10281 | GGAAAGATGA AACCTCGTTA TGAAATCAAC TCCCTGATTA |
| 10321 | GATACCACTG CAAAGATGGT TTCATTCAAC GTCACCTTCC |
| 10361 | AACTATCCGG TGCTTAGGAA ATGGAAGATG GGCTATACCT |
| 10401 | AAAATTACCT GCATGAACCC ATCTGCATAC CAAAGGACTT |
| 10441 | ATTCTATGAA ATACTTTAAA AATTCCTCAT CAGCAAAGGA |
| 10481 | CAATTCAATA AATACATCCA AACATGATCA TCGTTGGAGC |
| 10521 | CGGAGGTGGC AGGAGTCGAG GCGCTGATCC CTAAAATGGC |
| 10561 | GAACATGTGT TTTCATCATT TCAGCCAAAG TCCTAACTTC |
| 10601 | CTGTGCCTTT CCTATCACCT CGAGAAGTAA TTATCAGTTG |
| 10641 | GTTTGGATTT TTGGACCACC GTTCAGTCAT TTTGGGTTGC |
| 10681 | CGTGCTCCCA AAACATTTTA AATGAAAGTA TTGGCATTCA |
| 10721 | AAAAGACAGC AGACAAAATG AAAGAAAATG AGAGCAGAAA |
| 10761 | GTAAGCATTT CCAGCCTATC TAATTTCTTT AGTTTTCTAT |
| 10801 | TTGCCTCCAG TGCAGTCCAT TTCCTAATGT ATACCAGCCT |
| 10841 | ACTGTACTAT TTAAAATGCT CAATTTCAGC ACCGATGGCC |
| 10881 | ATGTAAATAA GATGATTTAA TGTTGATTTT AATCCTGTAT |
| 10921 | ATAAAATAAA AAGTCACAAT GAGTTTGGGC ATATTTAATG |
| 10961 | ATGATTATGG AGCCTTAGAG GTCTTTAATC ATTGGTTCGG |
| 11001 | CTGCTTTTAT GTAGTTTAGG CTGGAAATGG TTTCACTTGC |
| 11041 | TCTTTGACTG TCAGCAAGAC TGAAGATGGC TTTTCCTGGA |
| 11081 | CAGCTAGAAA ACACAAAATC TTGTAGGTCA TTGCACCTAT |
| 11121 | CTCAGCCATA GGTGCAGTTT GCTTCTACAT GATGCTAAAG |
| 11161 | GCTGCGAATG GGATCCTGAT GGAACTAAGG ACTCCAATGT |
| 11201 | CGAACTCTTC TTTGCTGCAT TCCTTTTTCT TCACTTACAA |
| 11241 | GAAAGGCCTG AATGGAGGAC TTTTCTGTAA CCAGGAACAT |
| 11281 | TTTTTAGGGG TCAAAGTGCT AATAATTAAC TCAACCAGGT |
| 11321 | CTACTTTTTA ATGGCTTTCA TAACACTAAC TCATAAGGTT |
| 11361 | ACCGATCAAT GCATTTCATA CGGATATAGA CCTAGGGCTC |
| 11401 | TGGAGGGTGG GGGATTGTTA AAACACATGC AAAAAAAAAA |
| 11441 | AAAAAAAAAA AAAAAGAAAT TTTGTATATA TAACCATTTT |
| 11481 | AATCTTTTAT AAAGTTTTGA ATGTTCATGT ATGAATGCTG |
| 11521 | CAGCTGTGAA GCATACATAA ATAAATGAAG TAAGCCATAC |
| 10561 | TGATTTAATT TATTGGATGT TATTTTCCCT AAGACCTGAA |
| 11601 | AATGAACATA GTATGCTAGT TATTTTTCAG TGTTAGCCTT |
| 11641 | TTACTTTCCT CACACAATTT GGAATCATAT AATATAGGTA |
| 11681 | CTTTGTCCCT GATTAAATAA TGTGACGGAT AGAATGCATC |
| 11721 | AAGTGTTTAT TATGAAAAGA GTGGAAAAGT ATATAGCTTT |
| 11761 | TAGCAAAAGG TGTTTGCCCA TTCTAAGAAA TGAGCGAATA |
| 11801 | TATAGAAATA GTGTGGGCAT TTCTTCCTGT TAGGTGGAGT |
| 11841 | GTATGTGTTG ACATTTCTCC CCATCTCTTC CCACTCTGTT |
| 11881 | TTCTCCCCAT TATTTGAATA AAGTGACTGC TGAAGATGAC |
| 11921 | TTTGAATCCT TATCCACTTA ATTTAATGTT TAAAGAAAAA |
| 11961 | CCTGTAATGG AAAGTAAGAC TCCTTCCCTA ATTTCAGTTT |
| 12001 | AGAGCAACTT GAAGAAGAGT AGACAAAAAA TAAAATGCAC |
| 12041 | ATAGAAAAAG AGAAAAAGGG CACAAAGGGA TTGGCCCAAT |
| 12081 | ATTGATTCTT TTTTTATAAA ACCTCCTTTG GCTTAGAAGG |
| 12121 | AATGACTCTA GCTACAATAA TACACAGTAT GTTTAAGCAG |
| 12161 | GTTCCCTTGG TTGTTGCATT AAATGTAATC CACCTTTAGG |
| 12201 | TATTTTAGAG CACAGAACAA CACTGTGTTG ATCTAGTAGG |
| 12241 | TTTCTATTTT TCCTTTCTCT TTACAATGCA CATAATACTT |
| 12281 | TCCTGTATTT ATATCATAAC GTGTATAGTG TAAAATGTGA |
| 12321 | ATGACTTTTT TTGTGAATGA AAATCTAAAA TCTTTGTAAC |
| 12361 | TTTTTATATC TGCTTTTGTT TCACCAAAGA AACCTAAAAT |
| 12401 | CCTTCTTTTA CTACAC |

Sequences for the versican core protein and nucleic acid encoding the core protein are also available, for example, in E. Ruoslahti, U.S. Pat. No. 5,180,808; Wight and Merrilees, US Patent Application 2004/0213762.

Any such sequences can be used a basis for generating versican inhibitors, including versican peptide inhibitors and versican inhibitory nucleic acids.

### Versican Inhibitors

“Versican inhibitor” as used herein may be any versican inhibitor, including but not limited to peptides, proteins (e.g., the V3 molecule or a truncated versican thereof without the polysaccharide, with a modified or truncated polysaccharide), small organic compounds, antibodies, inhibitory nucleic acids such as antisense nucleic acids, siRNAs, etc., as for example described further below or in Wight and Merrilees, Therapeutic Compounds and Methods, US Patent Application No. 2004/0213762 (Published Oct. 28, 2004). Thus the inhibitor can be a competitive inhibitor (e.g., exogenous V3 competing with bound versican for substrate), a direct inhibitor (e.g., antibody blocking the extracellular portion of versican to prevent binding), a down-regulator of versican expression or translation (e.g., an inhibitory nucleic acid), and the like. Examples of versican inhibitors include budesonide, hyaluronan oligomers, anti-versican antibodies, non-functioning versican peptides, inhibitory nucleic acids that target versican nucleic acids, and combinations thereof.

An inhibitor of versican can reduce the expression and/or activity of a versican by any amount such as, for example, by at least 2%, 5%, 10%, 20%, 40%, 50%, 60%, 70%, 80% or more than 80%.

In some embodiments, the versican inhibitor competitively inhibits the binding of versican to one or two or more of CD44, the EGF receptor, Tenascin R, PSGL-I, hyaluronan, fibronectin, and/or Apolipoprotein B-containing lipoproteins.

### Hyaluronan

Hyaluronan is an anionic, nonsulfated glycosaminoglycan with the following general structure:

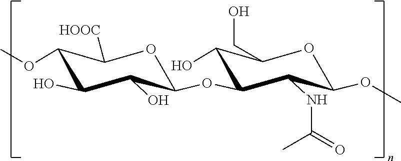

wherein n is an integer of about 2 to about 100,000. The value of n can vary. For example, n can range from 3 to 5000, from 4 to 500, from 4 to 100, from 4 to 50 or from 4 to 25.

### Peptide Inhibitors

Versican inhibitors include peptides that inhibit interactions between versican and other factors or receptors. For example, versican has a number of domains that can bind factors such as Toll-like receptor 2 (TLR2), hyaluronic acid, Hyaluronan and proteoglycan link protein 1 (HAPLN 1), aggrecan, tenascin R, fibulin-2, L-selectin, P-selectin, and CD44.

Versican peptides may act as competitive inhibitors that bind to factors such as Toll-like receptor 2 (TLR2), hyaluronic acid, hyaluronan, proteoglycan link protein 1 (HAPLN 1), aggrecan, tenascin R, fibulin-2, L-selectin, P-selectin, and CD44, but because the versican peptides lack other versican functions, those peptide inhibitors prevent endogenous versican from forming interactions that can contribute to the establishment and outgrowth of metastases.

For example, versican peptide inhibitors can have any of the following sequences, and/or any sequence with at least 90% sequence identity to any of the following sequences.

Versican peptide inhibitor sequence with amino acids: 21-146 (SEQ ID NO:4):

| | |
|---|---|
| 21 | LHKVKVGKSP PVRGSLSGKV |
| 41 | SLPCHFSTMP TLPPSYNTSE FLRIKWSKIE VDKNGKDLKE |
| 81 | TTVLVAQNGN IKIGQDYKGR VSVPTHPEAV GDASLTVVKL |
| 121 | LASDAGLYRC DVMYGIEDTQ DTVSLT |

Versican peptide inhibitor sequence with amino acids: 29-147 (SEQ ID NO:5):

| | |
|---|---|
| 29 | SP PVRGSLSGKV |
| 41 | SLPCHFSTMP TLPPSYNTSE FLRIKWSKIE VDKNGKDLKE |
| 81 | TTVLVAQNGN IKIGQDYKGR VSVPTHPEAV GDASLTVVKL |
| 121 | LASDAGLYRC DVMYGIEDTQ DTVSLTV |

Versican peptide inhibitor sequence with amino acids: 36-151 (SEQ ID NO:6):

| | |
|---|---|
| 1 | SGKV |
| 41 | SLPCHFSTMP TLPPSYNTSE FLRIKWSKIE VDKNGKDLKE |
| 81 | TTVLVAQNGN IKIGQDYKGR VSVPTHPEAV GDASLTVVKL |
| 121 | LASDAGLYRC DVMYGIEDTQ DTVSLTVDGV VFHYRAATSR |

Versican peptide inhibitor sequence with amino acids: 150-245 (SEQ ID NO:7):

| | |
|---|---|
| 121 | V VFHYRAATSR |
| 161 | YTLNFEAAQK ACLDVGAVIA TPEQLFAAYE DGFEQCDAGW |
| 201 | LADQTVRYPI RAPRVGCYGD KMGKAGVRTY GFRSPQETYD |
| 241 | VYCYV |

Versican peptide inhibitor sequence with amino acids: 150-244 (SEQ ID NO:8):

| | |
|---|---|
| 150 | V VFHYRAATSR |
| 161 | YTLNFEAAQK ACLDVGAVIA TPEQLFAAYE DGFEQCDAGW |
| 201 | LADQTVRYPI RAPRVGCYGD KMGKAGVRTY GFRSPQETYD |
| 241 | VYCY |

Versican peptide inhibitor sequence with amino acids: 150-170 (SEQ ID NO:9):

| | |
|---|---|
| 150 | V VFHYRAATSR |
| 161 | YTLNFEAAQK |

Versican peptide inhibitor sequence with amino acids: 181-320 (SEQ ID NO:10):

| | |
|---|---|
| 181 | TPEQLFAAYE DGFEQCDAGW |
| 201 | LADQTVRYPI RAPRVGCYGD KMGKAGVRTY GFRSPQETYD |
| 241 | VYCYVDHLDG DVFHLTVPSK FTFEEAAKEC ENQDARLATV |
| 281 | GELQAAWRNG FDQCDYGWLS DASVRHPVTV ARAQCGGGLL |

Versican peptide inhibitor sequence with amino acids: 251-347 (SEQ ID NO:11):

| | |
|---|---|
| 251 | DVFHLTVPSK FTFEEAAKEC ENQDARLATV |
| 281 | GELQAAWRNG FDQCDYGWLS DASVRHPVTV ARAQCGGGLL |
| 321 | GVRTLYRFEN QTGFPPPDSR FDAYCFK |

Versican peptide inhibitor sequence with amino acids: 251-346 (SEQ ID NO:12):

| | |
|---|---|
| 251 | DVFHLTVPSK FTFEEAAKEC ENQDARLATV |
| 281 | GELQAAWRNG FDQCDYGWLS DASVRHPVTV ARAQCGGGLL |
| 321 | GVRTLYRFEN QTGFPPPDSR FDAYCF |

Versican peptide inhibitor sequence with amino acids: 250-400 (SEQ ID NO:13):

| | |
|---|---|
| 250 | G DVFHLTVPSK FTFEEAAKEC ENQDARLATV |
| 281 | GELQAAWRNG FDQCDYGWLS DASVRHPVTV ARAQCGGGLL |
| 321 | GVRTLYRFEN QTGFPPPDSR FDAYCFKPKE ATTIDLSILA |
| 361 | ETASPSLSKE PQMVSDRTTP IIPLVDELPV IPTEFPPVGN |

Versican peptide inhibitor sequence with amino acids: 351-600 (SEQ ID NO:14):

| | |
|---|---|
| 321 | ATTIDLSILA |
| 361 | ETASPSLSKE PQMVSDRTTP IIPLVDELPV IPTEFPPVGN |
| 401 | IVSFEQKATV QPQAITDSLA TKLPTPTGST KKPWDMDDYS |
| 441 | PSASGPLGKL DISEIKEEVL QSTTGVSHYA TDSWDGVVED |
| 481 | KQTQESVTQI EQIEVGPLVT SMEILKHIPS KEFPVTETPL |
| 521 | VTARMILESK TEKKMVSTVS ELVTTGHYGF TLGEEDDEDR |
| 561 | TLTVGSDEST LIFDQIPEVI TVSKTSEDTI HTHLEDLESV |

Versican peptide inhibitor sequence with amino acids: 501-750 (SEQ ID NO:15):

| | |
|---|---|
| 481 | SMEILKHIPS KEFPVTETPL |
| 521 | VTARMILESK TEKKMVSTVS ELVTTGHYGF TLGEEDDEDR |
| 561 | TLTVGSDEST LIFDQIPEVI TVSKTSEDTI HTHLEDLESV |
| 601 | SASTTVSPLI MPDNNGSSMD DWEERQTSGR ITEEFLGKYL |
| 641 | STTPFPSQHR TEIELFPYSG DKILVEGIST VIYPSLQTEM |
| 681 | THRRERTETL IPEMRTDTYT DEIQEEITKS PFMGKTEEEV |
| 721 | FSGMKLSTSL SEPIHVTESS VEMTKSFDFP |

Versican peptide inhibitor sequence with amino acids: 721-900 (SEQ ID NO:16):

| | |
|---|---|
| 721 | FSGMKLSTSL SEPIHVTESS VEMTKSFDFP TLITKLSAEP |
| 761 | TEVRDMEEDF TATPGTTKYD ENITTVLLAH GTLSVEAATV |
| 801 | SKWSWDEDNT TSKPLESTEP SASSKLPPAL LTTVGMNGKD |
| 841 | KDIPSFTEDG ADEFTLIPDS TQKQLEEVTD EDIAAHGKFT |
| 881 | IRFQPTTSTG IAEKSTLRDS |

Versican peptide inhibitor sequence with amino acids: 851-1025 (SEQ ID NO:17):

| | |
|---|---|
| 841 | ADEFTLIPDS TQKQLEEVTD EDIAAHGKFT |
| 881 | IRFQPTTSTG IAEKSTLRDS TTEEKVPPIT STEGQVYATM |
| 921 | EGSALGEVED VDLSKPVSTV PQFAHTSEVE GLAFVSYSST |
| 961 | QEPTTYVDSS HTIPLSVIPK TDWGVLVPSV PSEDEVLGEP |
| 1001 | SQDILVIDQT RLEATISPET MRTTK |

Versican peptide inhibitor sequence with amino acids: 921-1151 (SEQ ID NO: 18):

| | |
|---|---|
| 921 | EGSALGEVED VDLSKPVSTV PQFAHTSEVE GLAFVSYSST |
| 961 | QEPTTYVDSS HTIPLSVIPK TDWGVLVPSV PSEDEVLGEP |
| 1001 | SQDILVIDQT RLEATISPET MRTTKITEGT TQEEFPWKEQ |
| 1041 | TAEKPVPALS STAWTPKEAV TPLDEQEGDG SAYTVSEDEL |
| 1081 | LTGSERVPVL ETTPVGKIDH SVSYPPGAVT EHKVKTDEVV |
| 1121 | TLTPRIGPKV SLSPGPEQKY ETEGSSTTGF T |

Versican peptide inhibitor sequence with amino acids: 1025-1301 (SEQ ID NO:19):

| | |
|---|---|
| 1001 | KITEGT TQEEFPWKEQ |
| 1041 | TAEKPVPALS STAWTPKEAV TPLDEQEGDG SAYTVSEDEL |
| 1081 | LTGSERVPVL ETTPVGKIDH SVSYPPGAVT EHKVKTDEVV |
| 1121 | TLTPRIGPKV SLSPGPEQKY ETEGSSTTGF TSSLSPFSTH |
| 1161 | ITQLMEETTT EKTSLEDIDL GSGLFEKPKA TELIEFSTIK |
| 1201 | VTVPSDITTA FSSVDRLHTT SAFKPSSAIT KKPPLIDREP |
| 1241 | GEETTSDMVI IGESTSHVPP TTLEDIVAKE TETDIDREYF |
| 1281 | TTSSPPATQP TRPPTVEDKE A |

Versican peptide inhibitor sequence with amino acids: 1201-1360 (SEQ ID NO:20):

| | |
|---|---|
| 1201 | VTVPSDITTA FSSVDRLHTT SAFKPSSAIT KKPPLIDREP |
| 1241 | GEETTSDMVI IGESTSHVPP TTLEDIVAKE TETDIDREYF |
| 1281 | TTSSPPATQP TRPPTVEDKE AFGPQALSTP QPPASTKFHP |
| 1321 | DINVYIIEVR ENKTGRMSDL SVIGHPIDSE SKEDEPCSEE |

Versican peptide inhibitor sequence with amino acids: 1336-3089 (SEQ ID NO:21):

| | |
|---|---|
| 1321 | RMSDL SVIGHPIDSE SKEDEPCSEE |
| 1361 | TDPVHDLMAE ILPEFPDIIE IDLYHSEENE EEEEECANAT |
| 1401 | DVTTTPSVQY INGKHLVTTV PKDPEAAEAR RGQFESVAPS |
| 1441 | QNFSDSSESD THPFVIAKTE LSTAVQPKES TETTESLEVT |
| 1481 | WKPETYPETS EHFSGGEPDV FPTVPFHEEF ESGTAKKGAE |
| 1521 | SVTERDTEVG HQAHEHTEPV SLFPEESSGE IAIDQESQKI |
| 1561 | AFARATEVTF GEEVEKSTSV TYTPTIVPSS ASAYVSEEEA |
| 1601 | VTLIGNPWPD DLLSTKESWV EATPRQVVEL SGSSSIPITE |
| 1641 | GSGEAEEDED TMFTMVTDLS QRNTTDTLIT LDTSRIITES |
| 1681 | FFEVPATTIY PVSEQPSAKV VPTKFVSETD TSEWISSTTV |
| 1721 | EEKKRKEEEG TTGTASTFEV YSSTQRSDQL ILPFELESPN |
| 1761 | VATSSDSGTR KSFMSLTTPT QSEREMTDST PVFTETNTLE |
| 1801 | NLGAQTTEHS SIHQPGVQEG LTTLPRSPAS VFMEQGSGEA |
| 1841 | AADPETTTVS SFSLNVEYAI QAEKEVAGTL SPHVETTFST |
| 1881 | EPTGLVLSTV MDRVVAENIT QTSREIVISE RLGEPNYGAE |
| 1921 | IRGFSTGFPL EEDFSGDFRE YSTVSHPIAK EETVMMEGSG |
| 1961 | DAAFRDTQTS PSTVPTSVHI SHISDSEGPS STMVSTSAFP |
| 2001 | WEEFTSSAEG SGEQLVTVSS SVVPVLPSAV QKFSGTASSI |
| 2041 | IDEGLGEVGT VNEIDRRSTI LPTAEVEGTK APVEKEEVKV |
| 2081 | SGTVSTNFPQ TIEPAKLWSR QEVNPVRQEI ESETTSEEQI |
| 2121 | QEEKSFESPQ NSPATEQTIF DSQTFTETEL KTTDYSVLTT |
| 2161 | KKTYSDDKEM KEEDTSLVNM STPDPDANGL ESYTTLPEAT |
| 2201 | EKSHFFLATA LVTESIPAEH VVTDSPIKKE ESTKHFPKGM |
| 2241 | RPTIQESDTE LLFSGLGSGE EVLPTLPTES VNFTEVEQIN |
| 2281 | NTLYPHTSQV ESTSSDKIED FNRMENVAKE VGPLVSQTDI |
| 2321 | FEGSGSVTST TLIEILSDTG AEGPTVAPLP FSTDIGHPQN |
| 2361 | QTVRWAEEIQ TSRPQTITEQ DSNKNSSTAE INETTTSSTD |
| 2401 | FLARAYGFEM AKEFVTSAPK PSDLYYEPSG EGSGEVDIVD |
| 2441 | SFHTSATTQA TRQESSTTFV SDGSLEKHPE VPSAKAVTAD |
| 2481 | GFPTVSVMLP LHSEQNKSSP DPTSTLSNTV SYERSTDGSF |
| 2521 | QDRFREFEDS TLKPNRKKPT ENIIIDLDKE DKDLILTITE |
| 2561 | STILEILPEL TSDKNTIIDI DHTKPVYEDI LGMQTDIDTE |
| 2601 | VPSEPHDSND ESNDDSTQVQ EIYEAAVNLS LTEETFEGSA |
| 2641 | DVLASYTQAT HDESMTYEDR SQLDHMGFHF TTGIPAPSTE |
| 2681 | TELDVLLPTA TSLPIPRKSA TVIPEIEGIK AEAKALDDMF |
| 2721 | ESSTLSDGQA IADQSEIIPT LGQFERTQEE YEDKKHAGPS |
| 2761 | FQPEFSSGAE EALVDHTPYL SIATTHLMDQ SVTEVPDVME |
| 2801 | GSNPPYYTDT TLAVSTFAKL SSQTPSSPLT IYSGSEASGH |
| 2841 | TEIPQPSALP GIDVGSSVMS PQDSFKEIHV NIEATFKPSS |
| 2881 | EEYLHITEPP SLSPDTKLEP SEDDGKPELL EEMEASPTEL |
| 2921 | IAVEGTEILQ DFQNKTDGQV SGEAIKMFPT IKTPEAGTVI |
| 2961 | TTADEIELEG ATQWPHSTSA SATYGVEAGV VPWLSPQTSE |
| 3001 | RPTLSSSPEI NPETQAALIR GQDSTIAASE QQVAARILDS |
| 3041 | NDQATVNPVE FNTEVATPPF SLLETSNETD FLIGINEESV |
| 3081 | EGTAIYLPG |

Versican peptide inhibitor sequence with amino acids: 1341-1880 (SEQ ID NO:22):

| | |
|---|---|
| 1321 | SVIGHPIDSE SKEDEPCSEE |
| 1361 | TDPVHDLMAE ILPEFPDIIE IDLYHSEENE EEEEECANAT |
| 1401 | DVTTTPSVQY INGKHLVTTV PKDPEAAEAR RGQFESVAPS |
| 1441 | QNFSDSSESD THPFVIAKTE LSTAVQPNES TETTESLEVT |
| 1481 | WKPETYPETS EHFSGGEPDV FPTVPFHEEF ESGTAKKGAE |
| 1521 | SVTERDTEVG HQAHEHTEPV SLFPEESSGE IAIDQESQKI |
| 1561 | AFARATEVTF GEEVEKSTSV TYTPTIVPSS ASAYVSEEEA |
| 1601 | VTLIGNPWPD DLLSTKESWV EATPRQVVEL SGSSSIPITE |
| 1641 | GSGEAEEDED TMFTMVTDLS QRNTTDTLIT LDTSRIITES |
| 1681 | FFEVPATTIY PVSEQPSAKV VPTKFVSETD TSEWISSTTV |
| 1721 | EEKKRKEEEG TTGTASTFEV YSSTQRSDQL ILPFELESPN |
| 1761 | VATSSDSGTR KSFMSLTTPT QSEREMTDST PVFTETNTLE |
| 1801 | NLGAQTTEHS SIHQPGVQEG LTTLPRSPAS VFMEQGSGEA |
| 1841 | AADPETTTVS SFSLNVEYAI QAEKEVAGTL SPHVETTFST |

Versican peptide inhibitor sequence with amino acids: 1761-2400 (SEQ ID NO:23):

| | |
|---|---|
| 1801 | NLGAQTTEHS SIHQPGVQEG LTTLPRSPAS VFMEQGSGEA |
| 1841 | AADPETTTVS SFSLNVEYAI QAEKEVAGTL SPHVETTFST |
| 1881 | EPTGLVLSTV MDRVVAENIT QTSREIVISE RLGEPNYGAE |
| 1921 | IRGFSTGFPL EEDFSGDFRE YSTVSHPIAK EETVMMEGSG |
| 1961 | DAAFRDTQTS PSTVPTSVHI SHISDSEGPS STMVSTSAFP |
| 2001 | WEEFTSSAEG SGEQLVTVSS SVVPVLPSAV QKFSGTASSI |
| 2041 | IDEGLGEVGT VNEIDRRSTI LPTAEVEGTK APVEKEEVKV |
| 2081 | SGTVSTNFPQ TIEPAKLWSR QEVNPVRQEI ESETTSEEQI |
| 2121 | QEEKSFESPQ NSPATEQTIF DSQTFTETEL KTTDYSVLTT |
| 2161 | KKTYSDDKEM KEEDTSLVNM STPDPDANGL ESYTTLPEAT |
| 2201 | EKSHFFLATA LVTESIPAEH VVTDSPIKKE ESTKHFPKGM |
| 2241 | RPTIQESDTE LLFSGLGSGE EVLPTLPTES VNFTEVEQIN |
| 2281 | NTLYPHTSQV ESTSSDKIED FNRMENVAKE VGPLVSQTDI |
| 2321 | FEGSGSVTST TLIEILSDTG AEGPTVAPLP FSTDIGHPQN |
| 2361 | QTVRWAEEIQ TSRPQTITEQ DSNKNSSTAE INETTTSSTD |

Versican peptide inhibitor sequence with amino acids: 2201-2800 (SEQ ID NO:24):

| | |
|---|---|
| 2201 | EKSHFFLATA LVTESIPAEH VVTDSPIKKE ESTKHFPKGM |
| 2241 | RPTIQESDTE LLFSGLGSGE EVLPTLPTES VNFTEVEQIN |
| 2281 | NTLYPHTSQV ESTSSDKIED FNRMENVAKE VGPLVSQTDI |
| 2321 | FEGSGSVTST TLIEILSDTG AEGPTVAPLP FSTDIGHPQN |
| 2361 | QTVRWAEEIQ TSRPQTITEQ DSNKNSSTAE INETTTSSTD |
| 2401 | FLARAYGFEM AKEFVTSAPK PSDLYYEPSG EGSGEVDIVD |
| 2441 | SFHTSATTQA TRQESSTTFV SDGSLEKHPE VPSAKAVTAD |
| 2481 | GFPTVSVMLP LHSEQNKSSP DPTSTLSNTV SYERSTDGSF |
| 2521 | QDRFREFEDS TLKPNRKKPT ENIIIDLDKE DKDLILTITE |
| 2561 | STILEILPEL TSDKNTIIDI DHTKPVYEDI LGMQTDIDTE |
| 2601 | VPSEPHDSND ESNDDSTQVQ EIYEAAVNLS LTEETFEGSA |
| 2641 | DVLASYTQAT HDESMTYEDR SQLDHMGFHF TTGIPAPSTE |
| 2681 | TELDVLLPTA TSLPIPRKSA TVIPEIEGIK AEAKALDDMF |
| 2721 | ESSTLSDGQA IADQSEIIPT LGQFERTQEE YEDKKHAGPS |
| 2761 | FQPEFSSGAE EALVDHTPYL SIATTHLMDQ SVTEVPDVME |

Versican peptide inhibitor sequence with amino acids: 2721-3089 (SEQ ID NO:25):

| | |
|---|---|
| 2721 | ESSTLSDGQA IADQSEIIPT LGQFERTQEE YEDKKHAGPS |
| 2761 | FQPEFSSGAE EALVDHTPYL SIATTHLMDQ SVTEVPDVME |
| 2801 | GSNPPYYTDT TLAVSTFAKL SSQTPSSPLT IYSGSEASGH |
| 2841 | TEIPQPSALP GIDVGSSVMS PQDSFKEIHV NIEATFKPSS |
| 2881 | EEYLHITEPP SLSPDTKLEP SEDDGKPELL EEMEASPTEL |
| 2921 | IAVEGTEILQ DFQNKTDGQV SGEAIKMFPT IKTPEAGTVI |
| 2961 | TTADEIELEG ATQWPHSTSA SATYGVEAGV VPWLSPQTSE |
| 3001 | RPTLSSSPEI NPETQAALIR GQDSTIAASE QQVAARILDS |
| 3041 | NDQATVNPVE FNTEVATPPF SLLETSNETD FLIGINEESV |
| 3081 | EGTAIYLPG |

Versican peptide inhibitor sequence with amino acids: 2709-2741 (SEQ ID NO:26):

| | |
|---|---|
| 2681 | IK AEAKALDDMF |
| 2721 | ESSTLSDGQA IADQSEIIPT L |

Versican peptide inhibitor sequence with amino acids: 3089-3125 (SEQ ID NO:27):

| | |
|---|---|
| 3081 | GP DRCKMNPCLN GGTCYPTETS YVCTCVPGYS |
| 3121 | GDQCE |

Versican peptide inhibitor sequence with amino acids: 3091-3125 (SEQ ID NO:28):

| | |
|---|---|
| 3081 | DRCKMNPCLN GGTCYPTETS YVCTCVPGYS |
| 3121 | GDQCE |

Versican peptide inhibitor sequence with amino acids: 3121-3200 (SEQ ID NO:29):

| | |
|---|---|
| 3121 | GDQCELDFDE CHSNPCRNGA TCVDGFNTFR CLCLPSYVGA |
| 3161 | LCEQDTETCD YGWHKFQGQC YKYFAHRRTW DAAERECRLQ |

Versican peptide inhibitor sequence with amino acids: 3081-3120 (SEQ ID NO:30):

| | |
|---|---|
| 3081 | EGTAIYLPGP DRCKMNPCLN GGTCYPTETS YVCTCVPGYS |

Versican peptide inhibitor sequence with amino acids: 3091-3120 (SEQ ID NO: 31:

| | |
|---|---|
| 3091 | DRCKMNPCLN GGTCYPTETS YVCTCVPGYS |

Versican peptide inhibitor sequence with amino acids: 3127-3163 (SEQ ID NO:32):

| | |
|---|---|
| 3121 | DFDE CHSNPCRNGA TCVDGFNTFR CLCLPSYVGA |
| 3161 | LCE |

Versican peptide inhibitor sequence with amino acids: 3127-3144 (SEQ ID NO:33):

| | |
|---|---|
| 3121 | DFDE CHSNPCRNGA TCVD |

Versican peptide inhibitor sequence with amino acids: 3169-3292 (SEQ ID NO:34):

| | |
|---|---|
| 3161 | CD YGWHKFQGQC YKYFAHRRTW DAAERECRLQ |
| 3201 | GAHLTSILSH EEQMFVNRVG HDYQWIGLND KMFEHDFRWT |
| 3241 | DGSTLQYENW RPNQPDSFFS AGEDCVVIIW HENGQWNDVP |
| 3281 | CNYHLTYTCK KG |

Versican peptide inhibitor sequence with amino acids: 3176-3290 (SEQ ID NO:35):

| | |
|---|---|
| 3161 | FQGQC YKYFAHRRTW DAAERECRLQ |
| 3201 | GAHLTSILSH EEQMFVNRVG HDYQWIGLND KMFEHDFRWT |
| 3241 | DGSTLQYENW RPNQPDSFFS AGEDCVVIIW HENGQWNDVP |
| 3281 | CNYHLTYTCK |

Versican peptide inhibitor sequence with amino acids: 3187-3284 (SEQ ID NO:36):

| | |
|---|---|
| 3161 | RRTW DAAERECRLQ |
| 3201 | GAHLTSILSH EEQMFVNRVG HDYQWIGLND KMFEHDFRWT |
| 3241 | DGSTLQYENW RPNQPDSFFS AGEDCVVIIW HENGQWNDVP |
| 3281 | CNYH |

Versican peptide inhibitor sequence with amino acids: 3230-3264 (SEQ ID NO:37):

| | |
|---|---|
| 3201 | G HDYQWIGLND KMFEHDFRWT |
| 3241 | DGSTLQYENW RPNQDSFFS AGED |

Versican peptide inhibitor sequence with amino acids: 3254-3278 (SEQ ID NO:38):

| | |
|---|---|
| 3254 | QPDSFFS AGEDCVVIIW HENGQWND |

Versican peptide inhibitor sequence with amino acids: 3294-3354 (SEQ ID NO:39):

| | |
|---|---|
| 3294 | VACGQPP VVENAKTFGK MKPRYEINSL |
| 3321 | IRYHCKDGFI QRHLPTIRCL GNGRWAIPKI TCMN |

Versican peptide inhibitor sequence with amino acids: 3296-3352 (SEQ ID NO:40):

| | |
|---|---|
| 3296 | CGQPP VVENAKTFGK MKPRYEINSL |
| 3321 | IRYHCKDGFI QRHLPTIRCL GNGRWAIPKI TC |

Versican peptide inhibitor sequence with amino acids: 3306-3326 (SEQ ID NO:41):

| | |
|---|---|
| 3306 | KTFGK MKPRYEINSL |
| 3321 | IRYHCK |

Versican peptide inhibitor sequence with amino acids: 3355-3396 (SEQ ID NO:42):

| | |
|---|---|
| 3355 | SAYQR |
| 3361 | TYSMKYFKNS SSAKDNSINT SKHDHRWSRR WQESRR |

and combinations thereof.

Thus, versican inhibitors can include peptides with any of SEQ ID Nos:4-42, and peptides with sequences having at least 90% sequence identity to any of SEQ ID Nos:4-42. In some embodiments, the versican inhibitors can include peptides with at least 95% sequence identity to any of SEQ ID Nos:4-42. In addition, the versican inhibitors can include peptides with sequences such as SEQ ID Nos:4-42 where 1-10 amino acids are missing from either the N-terminus or the C-terminus. In some embodiments, the versican inhibitor peptides have sequences with any of SEQ ID Nos:4-42, or sequences having at least 90% (or at least 95%) sequence identity to any of SEQ ID Nos:4-42, where about 1-5 amino acids can be missing from either or both of the N-terminus or the C-terminus.

### Inhibitory Nucleic Acids

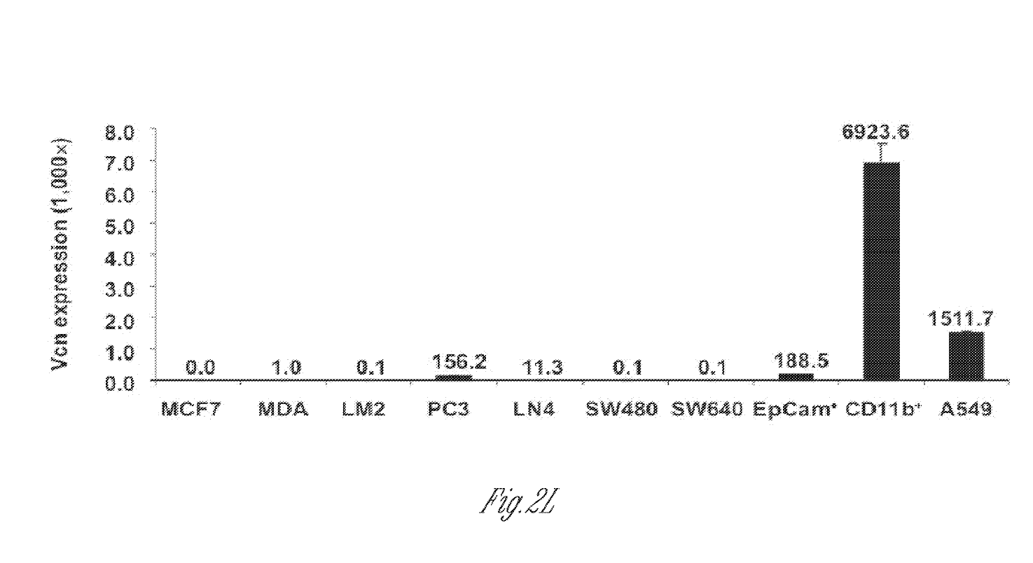

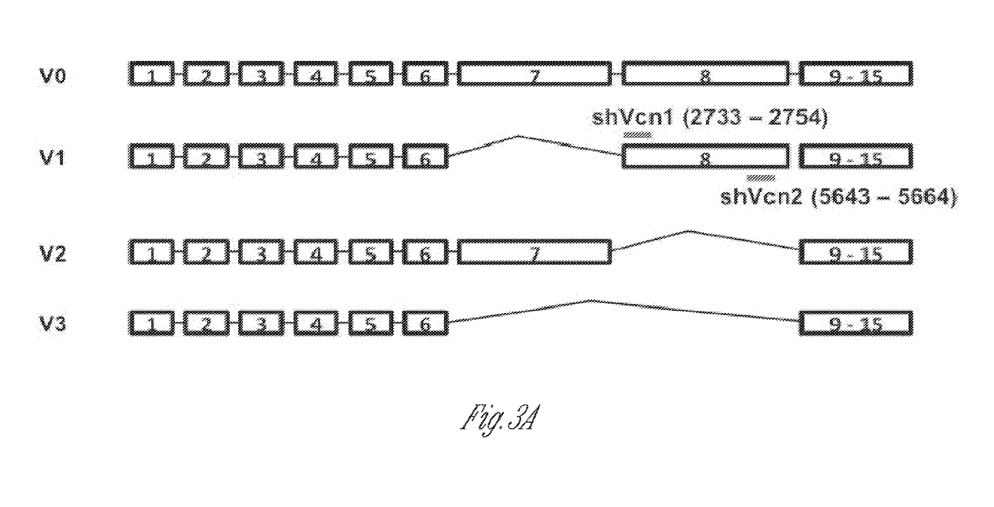

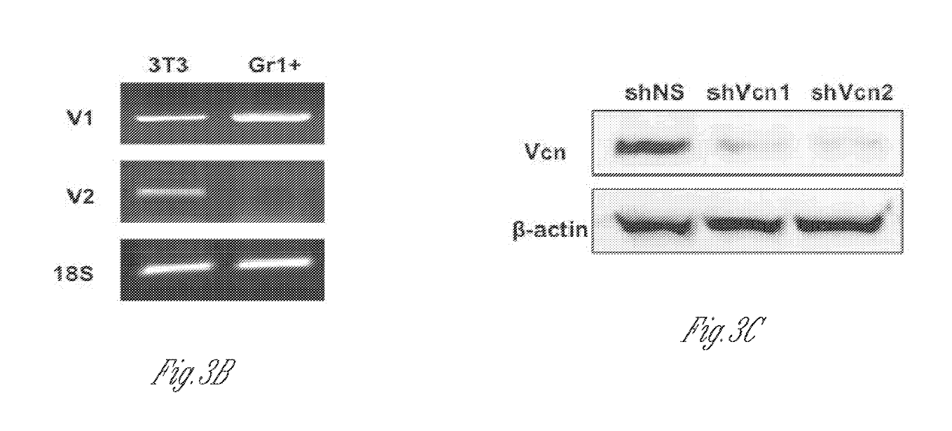

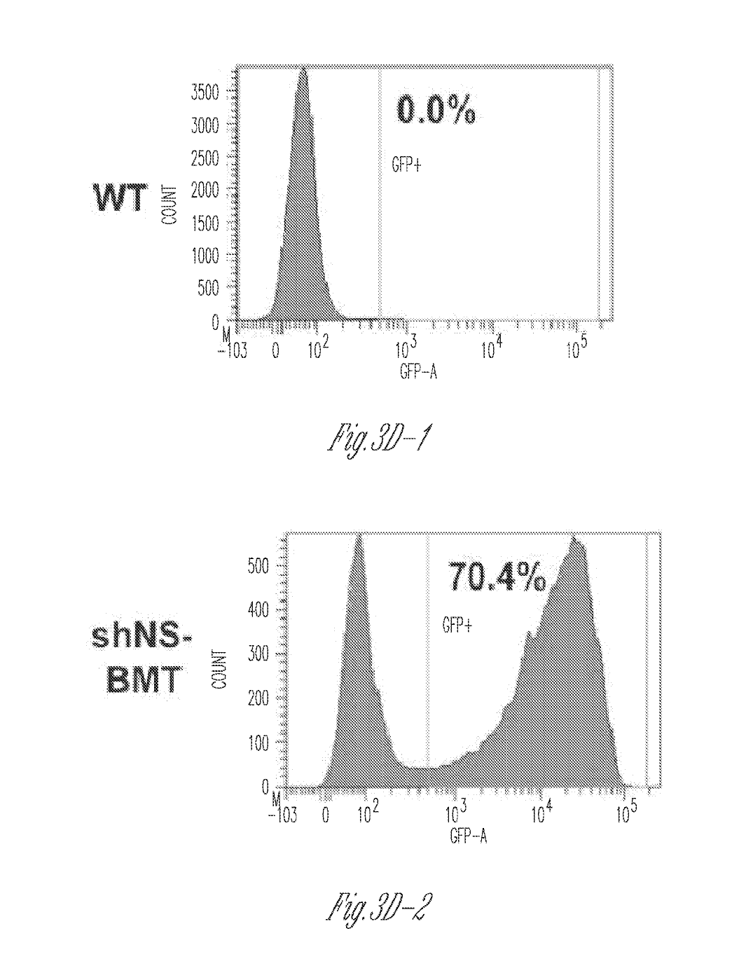

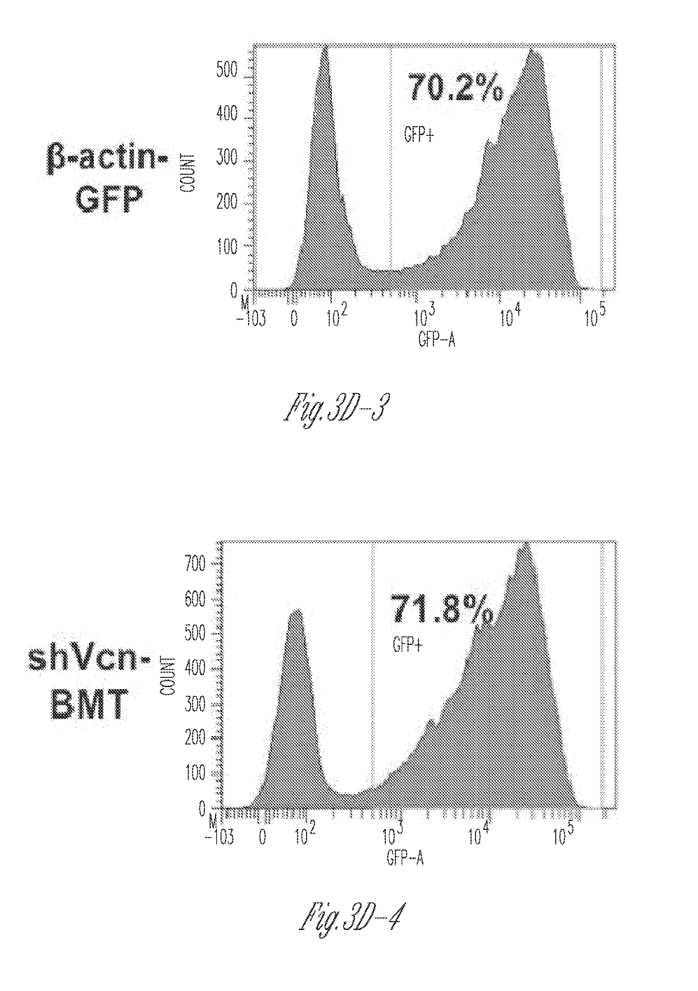

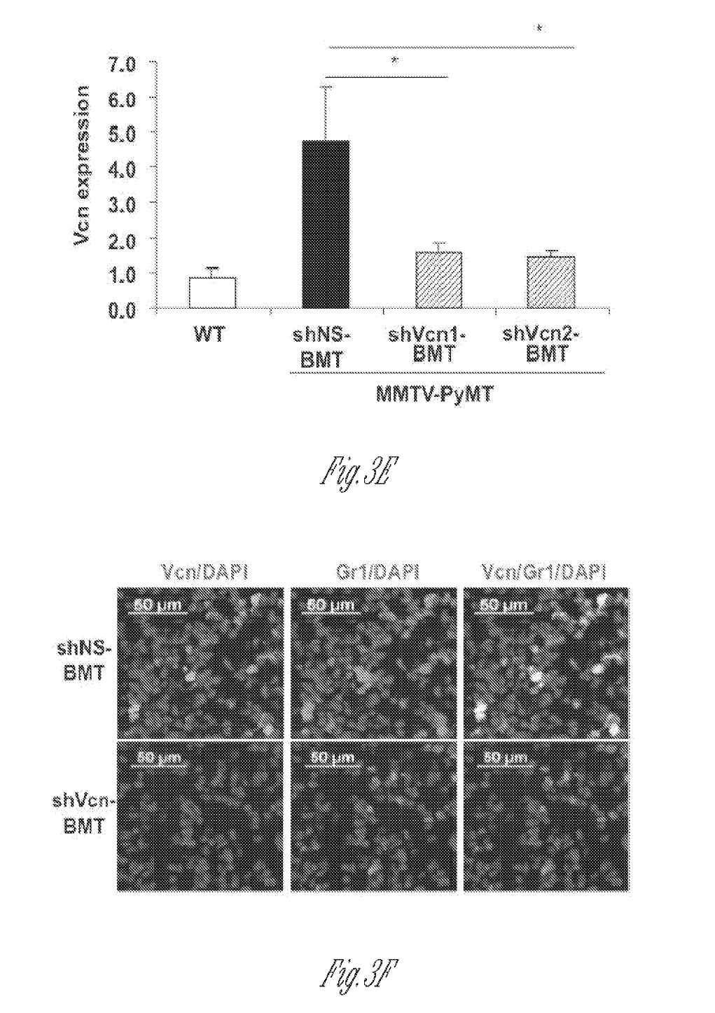

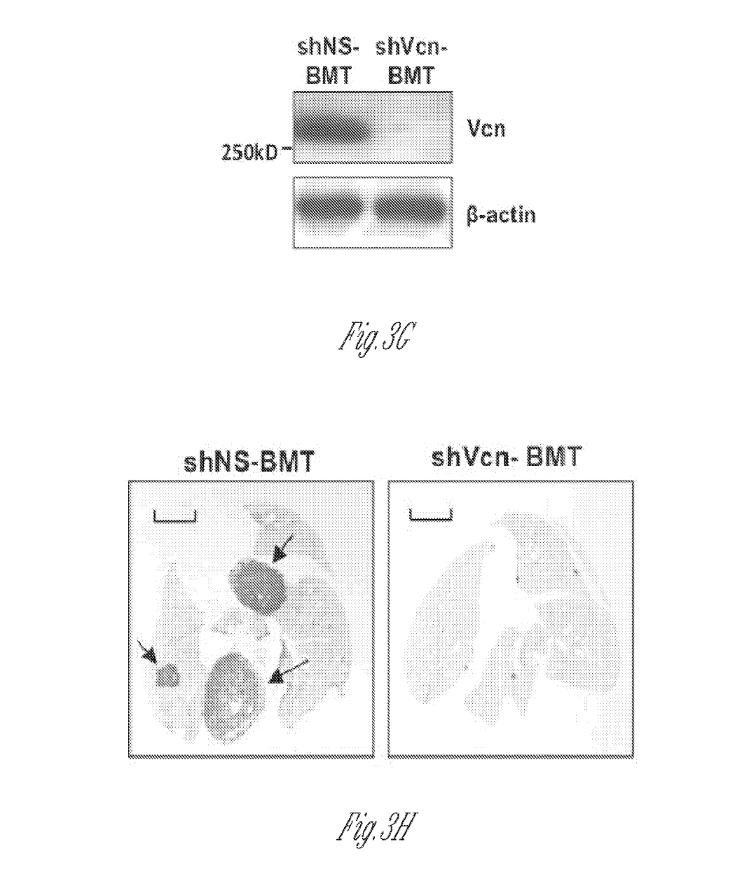

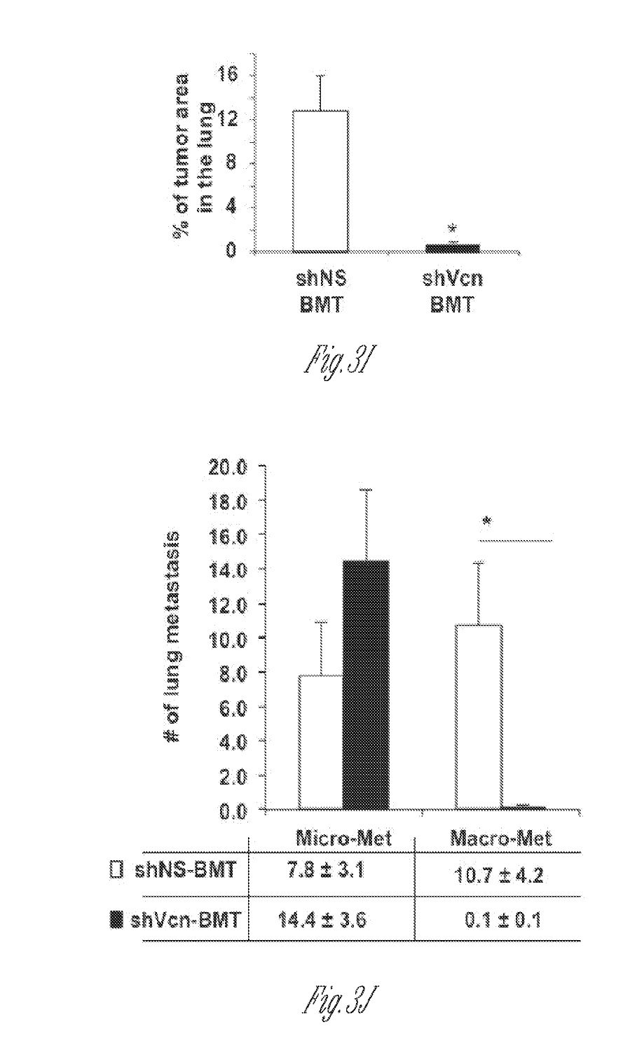

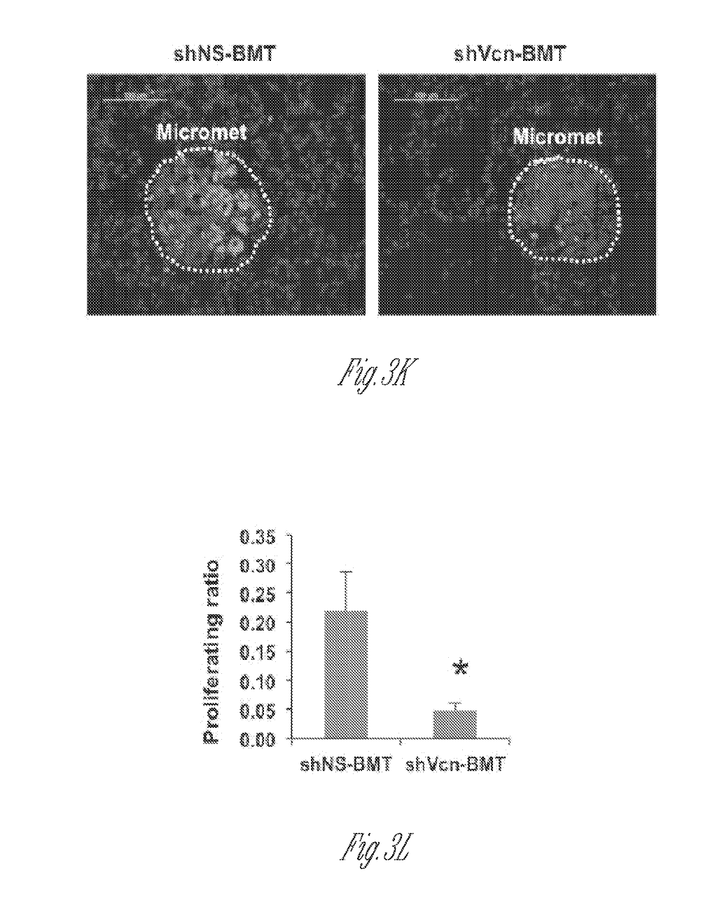

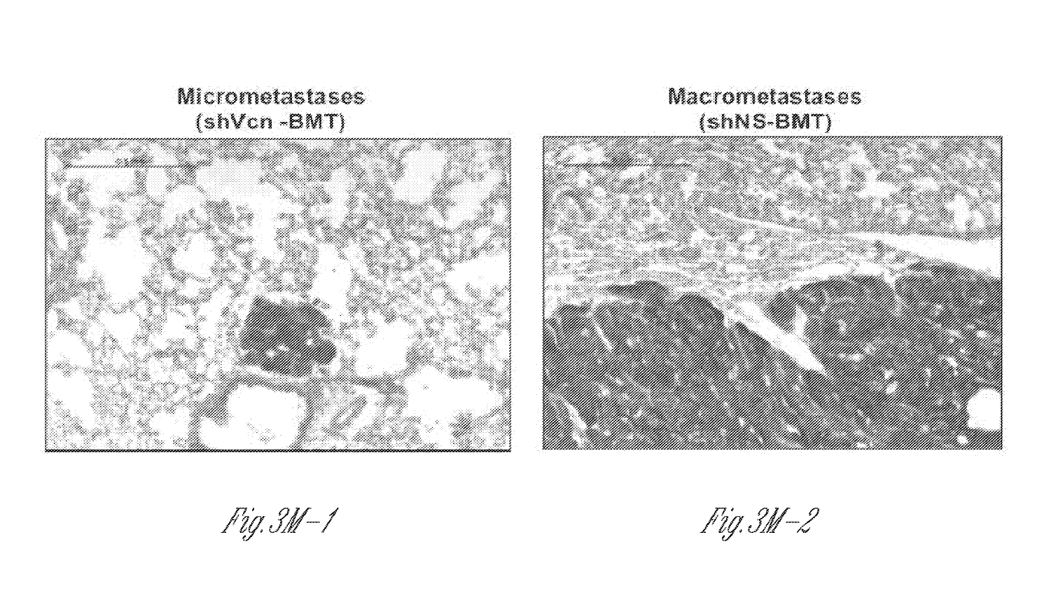

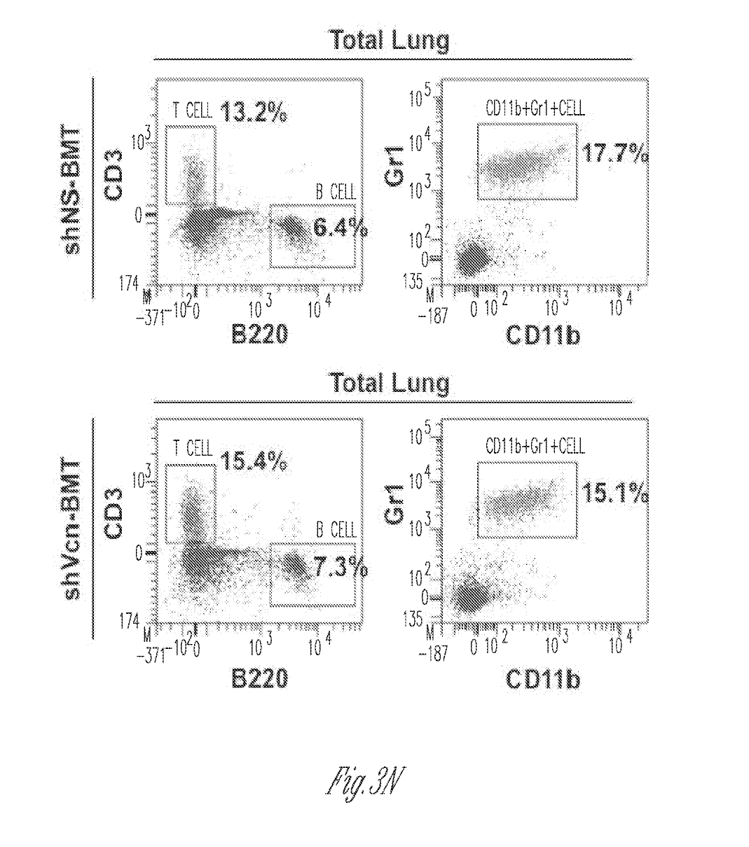

An inhibitor of versican can be an inhibitory nucleic acid with at least one segment that will hybridize to a versican nucleic acid under intracellular or stringent conditions. The inhibitory nucleic acid can reduce expression of a nucleic acid encoding versican. A nucleic acid encoding versican may be genomic DNA as well as messenger RNA. An inhibitory nucleic acid may be incorporated into a plasmid vector or viral DNA. It may be single strand or double strand, circular or linear. An example of a nucleic acid encoding versican is set forth in SEQ ID NO:3, which can be inhibited by inhibitory nucleic acids such as those with SEQ ID NO:43 or 44. See FIGS. 3A-3B and 3E. Versican-encoding nucleic acids to which inhibitory nucleic acids bind can also be a fragment of the sequences set forth in SEQ ID NO:3 provided that the versican-encoding nucleic acids encode a biologically active versican polypeptide.

An inhibitory nucleic acid is a polymer of ribose nucleotides or deoxyribose nucleotides having more than 13 nucleotides in length. An inhibitory nucleic acid may include naturally-occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a versican nucleic acid. Such an inhibitory nucleic acid may be completely complementary to a segment of the versican nucleic acid. Alternatively, some variability is permitted in the inhibitory nucleic acid sequences relative to versican sequences. An inhibitory nucleic acid can hybridize to a versican nucleic acid under intracellular conditions or under stringent hybridization conditions, and is sufficiently complementary to inhibit expression of a versican nucleic acid. Intracellular conditions refer to conditions such as temperature, pH and salt concentrations typically found inside a cell, e.g. an animal or mammalian cell. One example of such an animal or mammalian cell is a myeloid progenitor cell. Another example of such an animal or mammalian cell is a more differentiated cell derived from a myeloid progenitor cell. Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to a versican coding sequence, each separated by a stretch of contiguous nucleotides that are not complementary to adjacent coding sequences, can inhibit the function of a versican nucleic acid. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme or an antisense nucleic acid molecule.

Examples of versican inhibitory nucleic acids can include sequences such as SEQ ID NO:43 or SEQ ID NO:44. Versican inhibitory nucleic acids can include DNA or RNA sequences corresponding to SEQ ID NO:43 or SEQ ID NO:44, or DNA or RNA sequences that are complementary to SEQ ID NO:43 or SEQ ID NO:44.

The inhibitory nucleic acid molecule may be single or double stranded (e.g. a small interfering RNA (siRNA)), and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce versican translation such that the level of versican polypeptide is reduced. SiRNAs mediate post-transcriptional gene silencing in a sequence-specific manner. See, for example, website at invitrogen.com/site/us/enhome/Products-and-Services/Applications/rnai.html. Once incorporated into an RNA-induced silencing complex, siRNA mediate cleavage of the homologous endogenous mRNA transcript by guiding the complex to the homologous mRNA transcript, which is then cleaved by the complex. The siRNA may be homologous to any region of the versican mRNA transcript. The region of homology may be 30 nucleotides or less in length, preferable less than 25 nucleotides, and more preferably about 21 to 23 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411: 494-498 (2001); Harborth et al. Antisense Nucleic Acid Drug Dev. 13: 83-106 (2003).

The pSuppressor Neo vector for expressing hairpin siRNA. commercially available from IMGENEX (San Diego, Calif.), can be used to generate siRNA for inhibiting versican expression. The construction of the siRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002, 26(2): p. 199-213. Accordingly for synthesis of synthetic siRNA, a target region may be selected preferably 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA is 5′-AA(N19)UU, where N is any nucleotide in the mRNA sequence and should be approximately 50% G-C content. The selected sequence(s) can be compared to others in the human genome database to minimize homology to other known coding sequences (e.g., by Blast search, for example, through the NCBI website).

SiRNAs may be chemically synthesized, created by in vitro transcription, or expressed from an siRNA expression vector or a PCR expression cassette. See, e.g., website at invitrogen.com/site/us/en/home/Products-and-Services/Applications/rnai.html. When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA (SEQ ID NO:45). SiRNAs also may be produced in vivo by cleavage of double-stranded RNA introduced directly or via a transgene or virus. Amplification by an RNA-dependent RNA polymerase may occur in some organisms.

Examples of siRNA sequences that can hybridize to a versican nucleic acid include the following sequences and their complementary sequences:

(shVcn1; SEQ ID NO: 43)

5′-ACACCAGAATTAG AAAGTTCAA-3′

(shVcn2; SEQ ID NO: 44)

5′-AGCACCTTGTCTGATGGCCAAG-3′

An antisense oligonucleotide may also be used to specifically reduce versican expression, for example, by inhibiting transcription and/or translation. An antisense oligonucleotide is complementary to a sense nucleic acid encoding versican. For example, it may be complementary to the coding strand of a double-stranded cDNA molecule or complementary to a versican mRNA sequence. It may be complementary to an entire coding strand or to only a portion thereof. It may also be complementary to all or part of the noncoding region of a nucleic acid encoding versican. The non-coding region includes the 5′ and 3′ regions that flank the coding region, for example, the 5′ and 3′ untranslated sequences. An antisense oligonucleotide is generally at least six nucleotides in length, but may be about 8, 12, 15, 20, 25, 30, 35, 40, 45, or 50 nucleotides long. Longer oligonucleotides may also be used.

An inhibitory nucleic acid such as a short hairpin RNA siRNA or an antisense oligonucleotide may be prepared using methods known in the art, for example, by expression from an expression vector encoding the sequence of the inhibitory nucleic acid or from an expression cassette. Alternatively, it may be prepared by chemical synthesis using naturally-occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target versican nucleic acid.

Naturally-occurring nucleotides that can be employed in inhibitory nucleic acids include the ribose or deoxyribose nucleotides adenosine, guanine, cytosine, thymine and uracil. Examples of modified nucleotides that can be employed in inhibitory nucleic acids include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine. 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine. Thus, inhibitory nucleic acids may include modified nucleotides, as well as natural nucleotides such as combinations of ribose and deoxyribose nucleotides, and may be of any length discussed above and that can hybridize to a sense or antisense strand of a versican nucleic acid. For example, the inhibitory nucleic acid can be complementary to either strand of SEQ ID NO:3.

An inhibitory nucleic acid can also be a ribozyme. A ribozyme is an RNA molecule with catalytic activity and is capable of cleaving a single-stranded nucleic acid such as an mRNA that has a homologous region. See, for example, Cech, Science 236: 1532-1539 (1987); Cech, Ann. Rev. Biochem. 59:543-568 (1990); Cech, Curr. Opin. Struct. Biol. 2: 605-609 (1992); Couture and Stinchcomb, Trends Genet. 12: 510-515 (1996). A ribozyme may be used to catalytically cleave a versican mRNA transcript and thereby inhibit translation of the mRNA. See, for example, Haseloff et al., U.S. Pat. No. 5,641,673. A ribozyme having specificity for a versican nucleic acid may be designed based on the nucleotide sequence of any versican sequence, for example, SEQ ID NO:3. Methods of designing and constructing a ribozyme that can cleave an RNA molecule in trans in a highly sequence specific manner have been developed and described in the art. See, for example, Haseloff et al., Nature 334:585-591 (1988). A ribozyme may be targeted to a specific RNA by engineering a discrete “hybridization” region into the ribozyme. The hybridization region contains a sequence complementary to the target RNA that enables the ribozyme to specifically hybridize with the target. See, for example, Gerlach et al., EP 321,201. The target sequence may be a segment of at least about 5, 6, 7, 8, 9, 10, 12, 15, 17, 18, 19, 20, 21, 22, 23, 25, 30, or 50 contiguous nucleotides selected from a nucleotide sequence encoding a versican polypeptide, for example, SEQ ID NO:3. Longer complementary sequences may be used to increase the affinity of the hybridization sequence for the target. The hybridizing and cleavage regions of the ribozyme can be integrally related; thus, upon hybridizing to the target RNA through the complementary regions, the catalytic region of the ribozyme can cleave the target. Thus, an existing ribozyme may be modified to target versican by modifying the hybridization region of the ribozyme to include a sequence that is complementary to the target versican. Alternatively, an mRNA encoding versican may be used to select a catalytic RNA having a specific ribonuclease activity from a pool of RNA molecules. See, for example, Bartel & Szostak, Science 261:1411-1418 (1993).

### Smad2

Smad2 (also known as MADH2, MADR2, hMAD2 and JV18-1) is a member of a subgroup of Smad family transcription factors which are regulated by TGF-β and activins. Upon ligand binding Smad2 becomes phosphorylated and associates with Smad3. This complex then associates with Smad4 and translocates to the nucleus where it effects transcription of target genes. It has been demonstrated that the phosphorylation of Smad2 is necessary for the association with Smad4 (Souchelnytskyi et al., J. Biol. Chem., 1997, 272, 28107-28115) and that Smad2 and Smad4 interact with CREB binding protein, an essential component of the mammalian transcription apparatus (Topper et al., Proc. Natl. Acad. Sci. U.S.A., 1998, 95, 9506-9511).

The Smad2 gene is located on chromosome 18q21, a region that frequently undergoes allelic loss in many cancers. A missense somatic mutation and a 9-bp in-frame deletion were detected in the highly conserved region of JV 8-1 among 57 lung cancer specimens taken directly from patients (Uchida et al., Cancer Res., 1996, 56, 5583-5585). In addition, missense and nonsense mutations of the Smad2 gene have also been found in 6-17% of colorectal carcinoma cell lines and primary tumors (Eppert et al., Cell, 1996, 86, 543-552).

In normal cells, Smad2 acts to transmit signals from TGF-β and the activins. It has also been shown to mediate cross-talk between receptor tyrosine kinase pathways and receptor serine/threonine kinase pathways by acting as a positive effector in the EGF and HGF signalling cascades (de Caestecker et al., Genes Dev., 1998, 12, 1587-1592).

### Epithelial Cells

Epithelial cell biomarkers include E-cadherin, CDH1 promoter activity, occludin, B-catenin, cytokeratin 8, cytokeratin 18, P-cadherin and/or erbB3.

### Mesenchymal Cells

Mesenchymal cell biomarkers can include vimentin, fibronectin, N-cadherin, CDH1 methylation, zeb1, twist, FOXC2 and/or snail.

Agents that Mediate MET

Factor that may contribute to mesenchymal to epithelial transition include expression of the proangiogenic factor BV8 (Kowanez et al., Proc Natl Acad Sci USA 107:21248-55 (2010)), metastasis-promoting lysyl oxidase and matrix metalloproteinase 9 (MMP9; Erler et al., Cancer Cell 15:35-44 (2009)), agents that contribute to TGF-β-mediated metastasis (Yang et al., Cancer Cell 13:23-35 (2008)), and immune tolerance and suppression by virtue of innate MDSC activity (Ostrand-Rosenberg & Sinha, J Immunol 182:4499-506 (2009); Youn et al., J Immunol 181: 5791-802 (2008)).

Agents that Mediate EMT

Protein ligands that induce an epithelial-to-mesenchymal transition (EMT) in CFPAC-1 cells can be selected from OSM; HGF; BMP7; IGF2; LIF; PAR2 agonist SLIGKV-NH2; IL-33; PAR4 agonist AYPGKF-NH2; CTGF; and BMP4. CFPAC-1 is an epithelial tumor cell line.

### Metastatic Cancer Treatment

According to the invention, versican inhibitors are useful for preventing, treating and/or diagnosing metastatic cancer. Thus, one aspect of the invention is a method of treating or inhibiting the establishment and/or growth metastatic tumors in an animal, where the metastatic tumors are at distal sites from a primary tumor site within the animal. Such a method involves administering a versican inhibitor to the animal to thereby treat or inhibit the establishment and/or growth of metastatic tumors in an animal. Both human and veterinary uses are contemplated.

In some embodiments, the treatment and/or inhibition of metastatic tumors is performed on an animal where the primary tumor has been removed. In general, versican inhibition does not substantially affect primary tumors. Instead, inhibition of versican inhibition prevents or substantially inhibits the establishment and growth of metastases.

The methods of treating or inhibiting the establishment and/or growth metastatic tumors in an animal can include administering to a subject animal (e.g., a human), a therapeutically effective amount of a versican inhibitor. Such methods can also include surgery to remove the primary tumor and any metastatic tumors that are located, either before or during treatment with versican inhibitors. The methods of treating or inhibiting the establishment and/or growth metastatic tumors in an animal can also include administering a versican inhibitor with one or more other anti-cancer or chemotherapeutic agents.

In some embodiments, the methods can also include a detection step to ascertain whether the animal has metastatic tumors or is in need of treatment to inhibit the development of metastatic tumors. Such a detection step can include any of the versican detection procedures described herein. For example, a test sample from the animal can be tested to determine whether the test sample expressed at least about two-fold more versican than a control non-metastatic cancer sample.

The term “animal” as used herein, refers to an animal, such as a warm-blooded animal, which is susceptible to or has a disease associated with protease expression, for example, cancer. Mammals include cattle, buffalo, sheep. goats, pigs, horses, dogs, cats, rats, rabbits, mice, and humans. Also included are other livestock, domesticated animals and captive animals. The term “farm animals” includes chickens, turkeys, fish, and other farmed animals. Mammals and other animals including birds may be treated by the methods and compositions described and claimed herein. In some embodiments, the animal is a human.

As used herein, the term “cancer” includes solid animal tumors as well as hematological malignancies. The terms “tumor cell(s)” and “cancer cell(s)” are used interchangeably herein.

“Solid animal tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. In addition, a metastatic cancer at any stage of progression can be treated, such as micrometastatic tumors, megametastatic tumors, and recurrent cancers.

The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS.

The invention can also be used to treat cancer of the adrenal cortex, cancer of the cervix, cancer of the endometrium, cancer of the esophagus, cancer of the head and neck, cancer of the liver, cancer of the pancreas, cancer of the prostate, cancer of the thymus, carcinoid tumors, chronic lymphocytic leukemia, Ewing's sarcoma, gestational trophoblastic tumors, hepatoblastoma, multiple myeloma, non-small cell lung cancer, retinoblastoma, or tumors in the ovaries. A cancer at any stage of progression can be treated or detected, such as primary, metastatic, and recurrent cancers. Information regarding numerous types of cancer can be found, e.g., from the American Cancer Society (www.cancer.org), or from, e.g., Wilson et al. (1991) Harrison's Principles of Internal Medicine, 12th Edition, McGraw-Hill, Inc.

Treatment of, or treating, metastatic cancer can include the reduction in growth or the reduction in establishment of at least one metastatic tumor. The treatment also includes alleviation or diminishment of more than one symptom of metastatic cancer such as coughing, shortness of breath, hemoptysis, lymphadenopathy, enlarged liver, nausea, jaundice, bone pain, bone fractures, headaches, seizures, systemic pain and combinations thereof. The treatment may cure the cancer, e.g., it may prevent metastatic cancer, it may substantially eliminate metastatic tumor formation and growth, and/or it may arrest or inhibit the growth of metastatic tumors.

Anti-cancer activity can be evaluated against varieties of cancers using methods available to one of skill in the art. Anti-cancer activity, for example, is determined by identifying the lethal dose (LD 100) or the 50% effective dose (ED50) or the dose that inhibits growth at 50% (GI50) of an agent of the present invention that prevents the growth of a cancer. In one aspect, anti-cancer activity is the amount of the methods reduce 50%, 60%, 70%, 80%, 90%, 95%, 97%, 98%, 99% or 100% of metastases, for example, when measured by detecting versican expression at sites distal from a primary tumor site, or when assessed using available methods for detecting metastases.

### Metastatic Cancer Detection

As illustrated herein, the existence, extent, location and/or pathologic progression of metastatic cancer within an animal can be detected by detection of versican expression. Versican expression can be detected, for example, by use of a versican binding agent (e.g., an inhibitor or antibody) capable of binding to versican. Such a binding agent can provide information regarding the location, shape, extent and pattern of the metastatic tumor. A reporter molecule, label or signaling compound can be attached to versican binding agents to form a labeled versican binding agent. Such labeled versican binding agents can then be used in vivo or in vitro to image, locate or otherwise detect the tissue to which the agent binds and thereby detect the presence, location, shape, extent and pattern of the metastatic tumor.

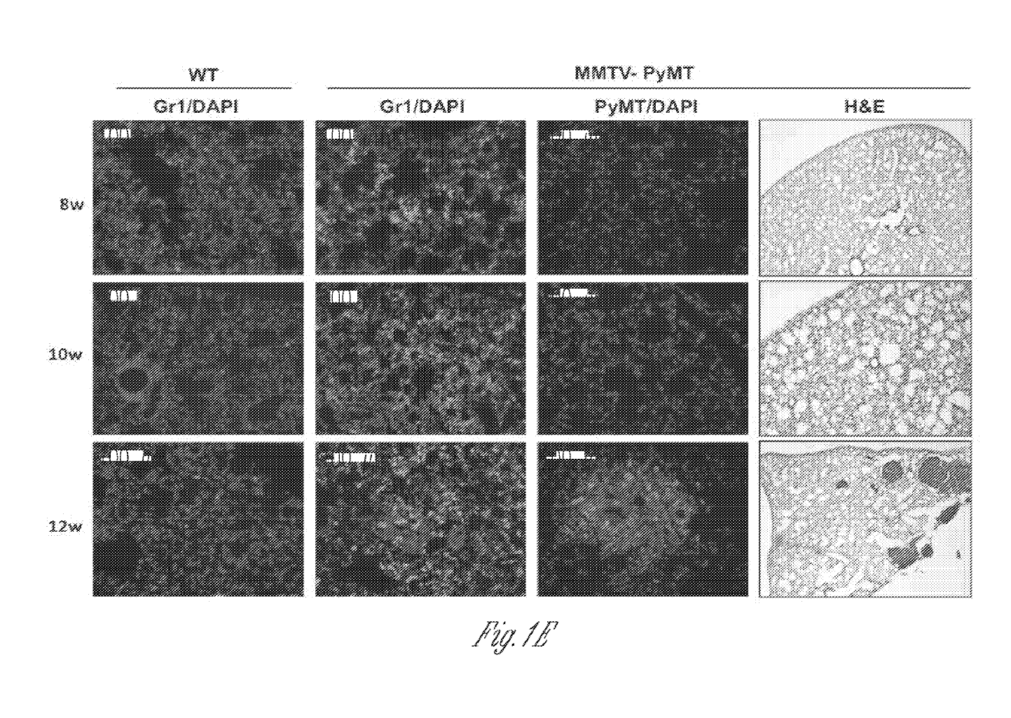

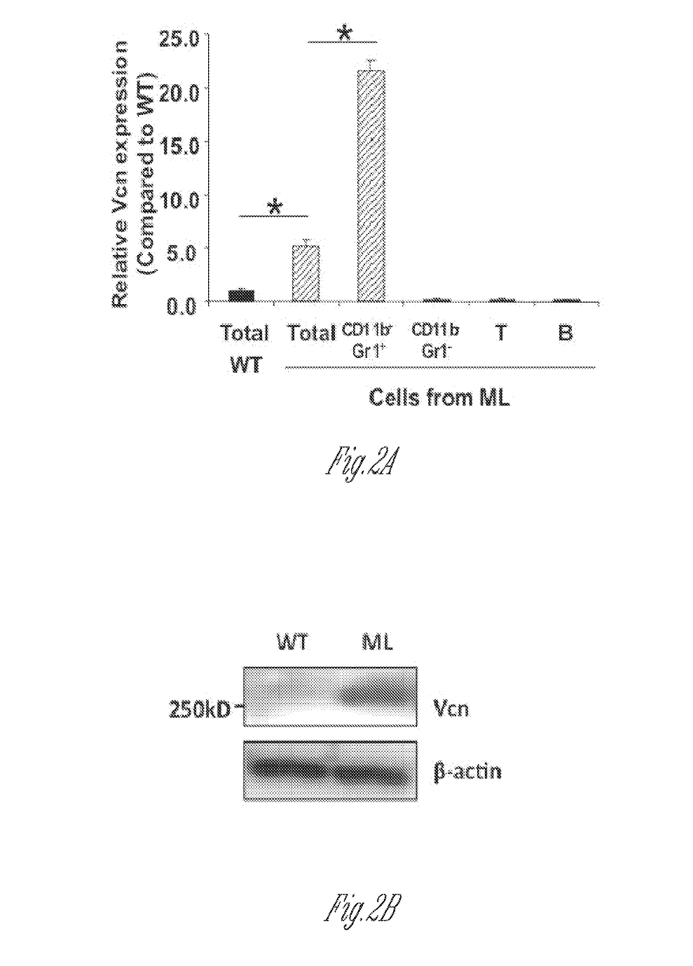

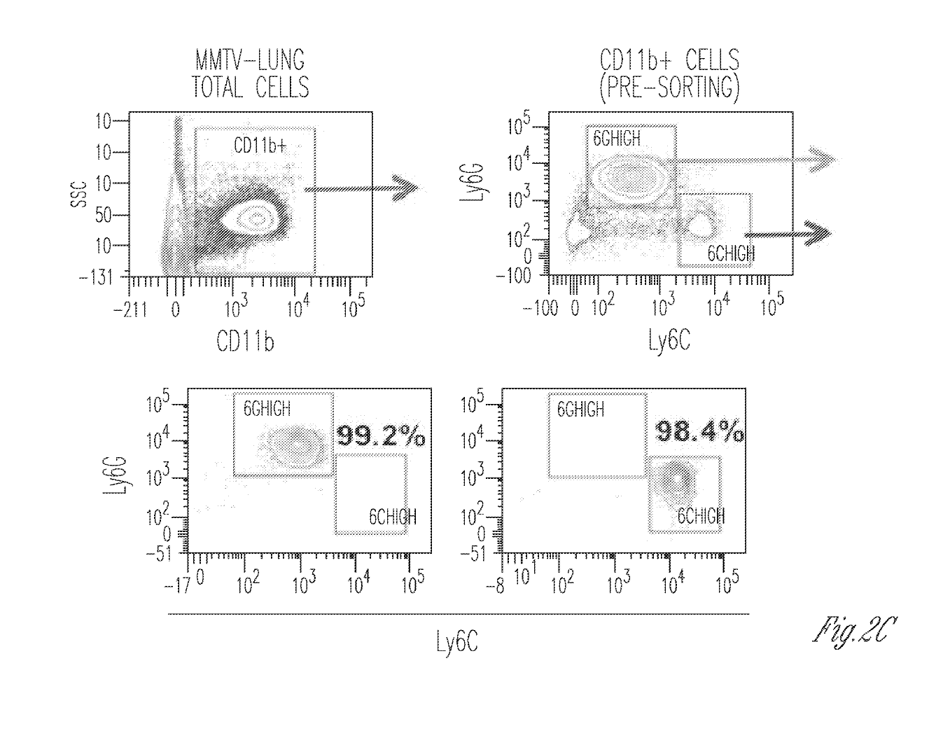

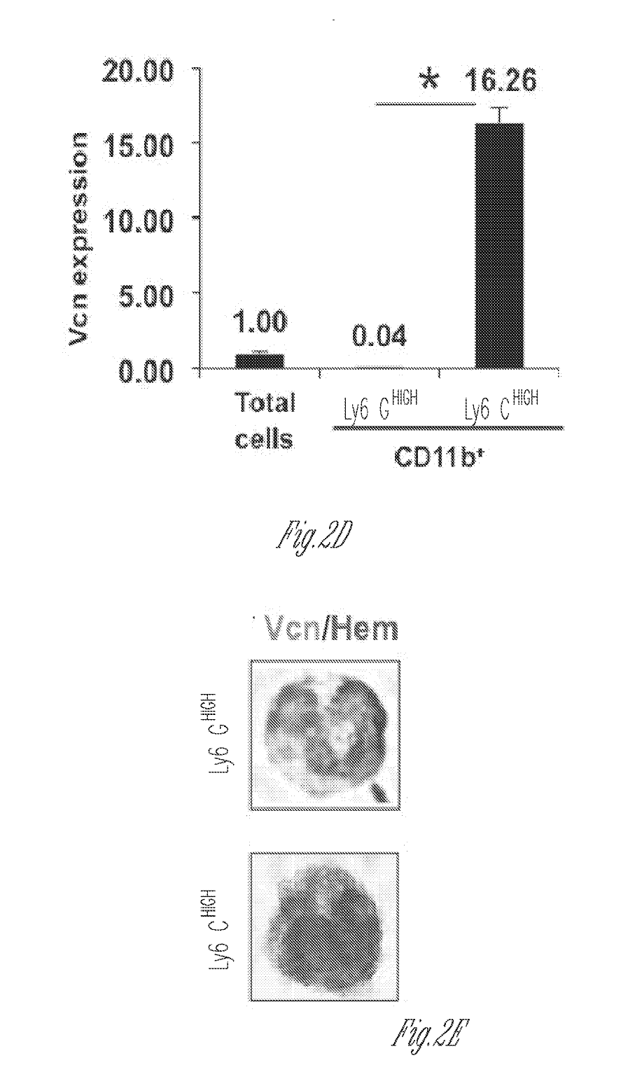

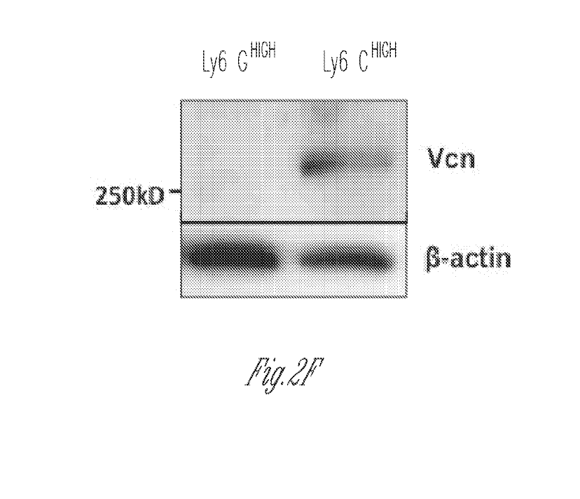

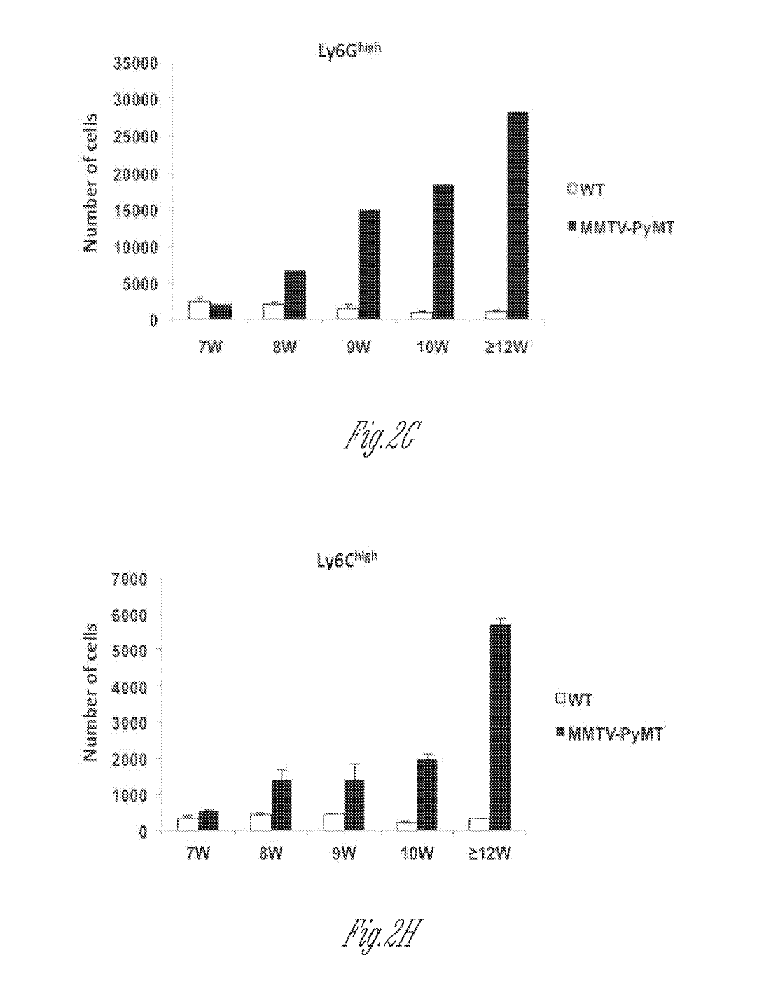

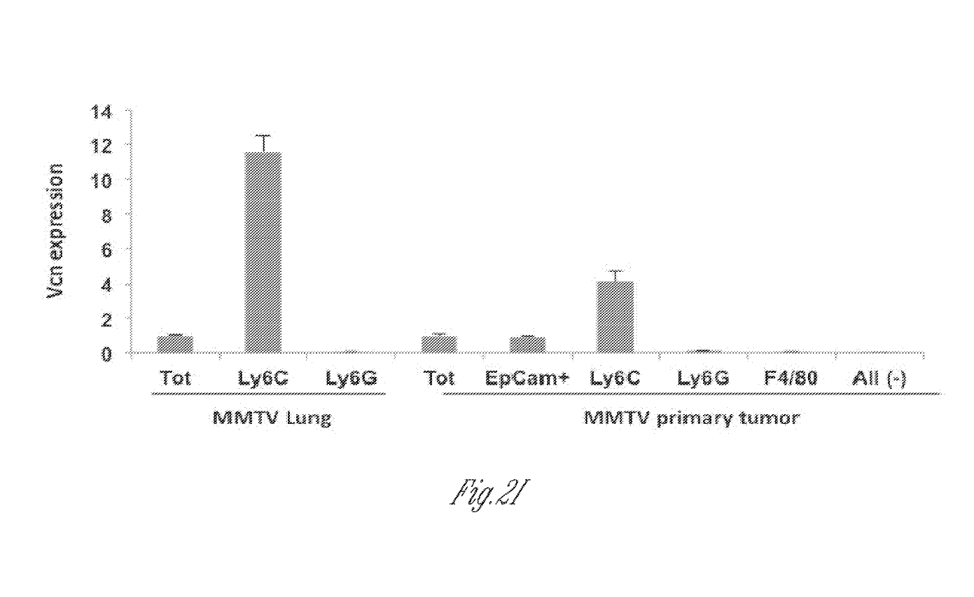

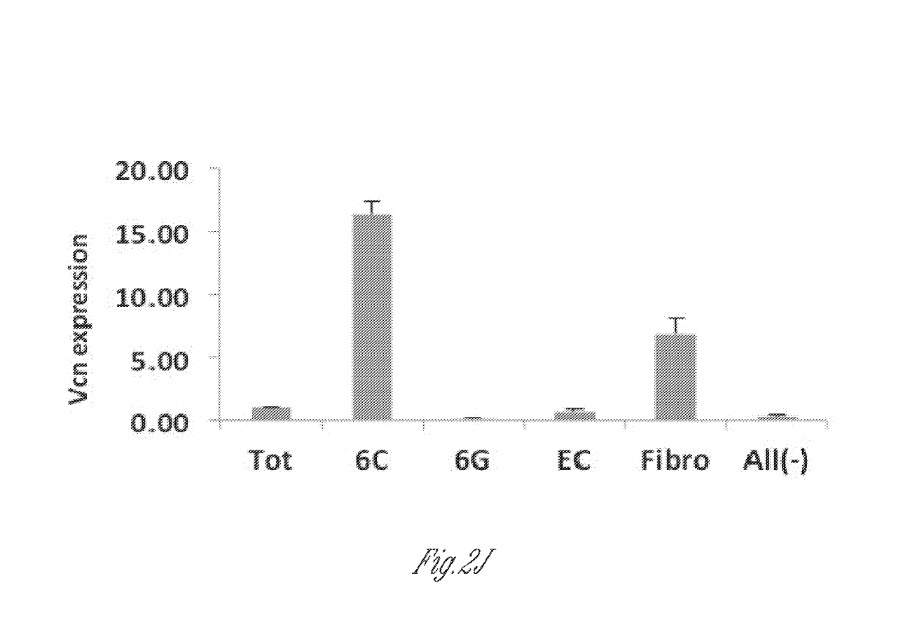

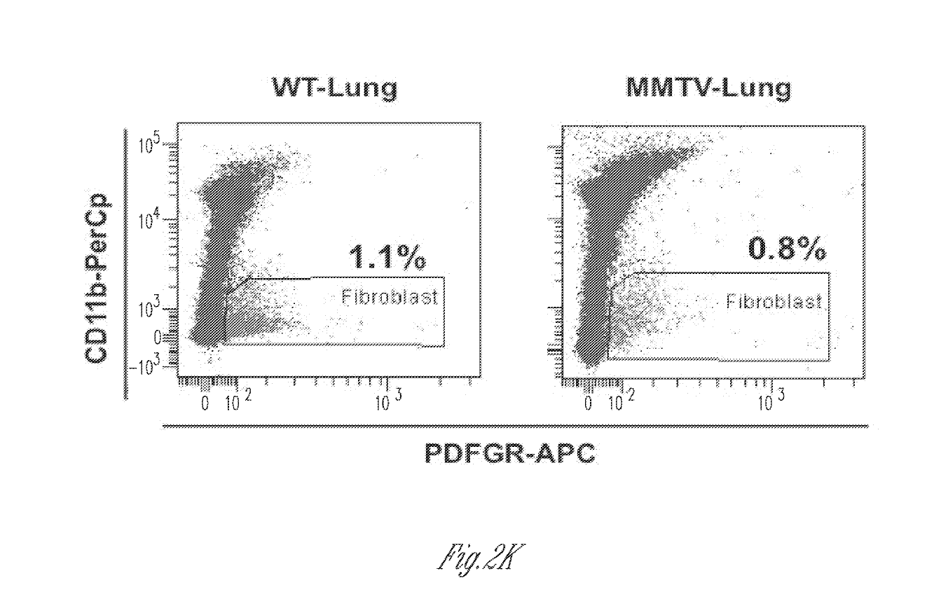

Thus, in one embodiment, the invention relates to a method of detecting metastatic cancer in an animal that includes testing whether a test sample from the animal expresses at least two-fold higher levels of versican than a control sample, where the control sample is a non-metastatic cancer sample. In another embodiment, the invention relates to a method of detecting metastatic cancer in an animal that includes administering a versican binding agent to the animal and observing whether the versican binding agent localizes to the region in the animal and emits a signal that is at least two-fold above a background signal from the binding agent. As illustrated herein, expression of at least two-fold higher levels of versican expression is evidence that a test sample contains metastatic tissues or that the animal has metastatic tumor tissues that are actively progressing and/or growing (FIGS. 2, 3 and 5, especially FIG. 2I, 2J, 3H-3M, 5H, 5I).

In some embodiments, the control non-metastatic cell is a normal cell. As used herein the terms “normal mammalian cell” and “normal animal cell” are defined as a cell that is growing under normal growth control mechanisms (e.g., genetic control) and that displays normal cellular differentiation and normal migration patterns. Cancer cells differ from normal cells in their growth patterns, migration and in the nature of their cell surfaces. For example cancer cells tend to grow continuously and chaotically, without regard for their neighbors, and metastatic cancer cells can migrate to distal sites to generate tumors in other areas of the body.

The test sample can be a tissue sample or a bodily fluid. For example, the test sample can be a biopsy sample of a tissue site suspected of harboring metastatic cells. Alternatively, the test sample can be a bodily fluid such as a serum sample, a plasma sample, a blood sample, a urine sample, a breast milk sample, a lymph sample, or a combination thereof.

The reporter molecule, label or signaling compound that is linked to the binding agent depends on the ultimate application (e.g., in vitro or in vivo detection of metastasis). For example, in some embodiments, the test sample is a body fluid or a tissue sample that is obtained from an animal and tested in vitro. In other embodiments, the binding agent is administered to the animal and a signal from a label on the binding agent is detected within the animal.

Labels employed with the binding agents of the invention can be fluorophores, radioisotopes, metals, enzymes, enzyme substrates, luminescent moieties, and the like. Where the aim is to detect metastatic cancer in vivo and/or to provide an image of the tumor, one of skill in the art may desire to use a diagnostic agent that is detectable in vivo, such as a paramagnetic, radioactive or fluorogenic agent. Such agents are available in the art, for example, as described and disclosed in U.S. Pat. No. 6,051,230, which is incorporated by reference herein in its entirety. Many diagnostic agents are known in the art to be useful for in vivo detection and/or imaging purposes, as are methods for their attachment to peptides and antibodies (see, e.g., U.S. Pat. Nos. 5,021,236 and 4,472,509, both incorporated herein by reference.

In the case of paramagnetic ions, one of skill may choose to use, for example, ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being preferred. One example of a label that may be used is gadolinium or a gadolinium complex. For example, the following gadolinium complex can be used as a label:

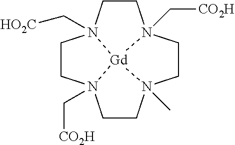

In some embodiments, boron may also be used a label. When boron-10 and gadolinium-157 are used in such a boron-gadolinium bifunctional targeting entity, tumors can be detected using magnetic resonance imaging (MRI) and then treated using the methods and versican inhibitors described herein. Such treatment can also be combined with neutron capture therapy. See, e.g., Takahashi et al., Synthesis and in vivo biodistribution of BPA-Gd-DTPA complex as a potential MRI contrast carrier for neutron capture therapy, BIOORGANIC & MEDICINAL CHEMISTRY 13: 735-743 (2005). Hence, this bifunctional boron-gadolinium agent is useful for treating cancers as well as detecting them.

Ions useful in other contexts, such as X-ray imaging, include but are not limited to lanthanum (III), gold (III), lead (II), and especially bismuth (III). Moreover, in the case of radioactive isotopes for therapeutic and/or diagnostic application, one might mention iodine¹³¹, iodine²³, iodine¹²⁵, technicium⁹⁹, indium¹¹¹, phosphorus³², rhenium¹⁸⁸, rhenium¹⁸⁶, gallium⁶⁷, sulfur³⁵, copper⁶⁷, yttrium⁹⁰, tritium³ or astatine²¹¹.

In some embodiments, binding agents may be conjugated with a dye or fluorescent moiety or intermediate such as biotin. Such conjugates can, for example, be used with infrared spectroscopy to detect and locate the tissues to which the agents bind.

An in vitro assay for identifying a metastatic tumor that expresses versican can include incubating a test sample under conditions that permit binding of any versican in the sample to a binding agent, and measuring whether such binding has occurred. In some embodiments, the extent of binding between the binding agent and versican may be detected, for example, by a label present on the binding agent or by a label that can bind to the binding agent. Such information may be used to detect and assess the extent, spread or size of a metastatic tumor. A reporter molecule can be attached to any molecule that stably binds to versican, where the reporter molecule can be detected in vitro or in vivo.

For example, the reporter molecule can be attached to one of the versican inhibitors described herein or to an antibody that binds to versican, where the inhibitor or antibody can be labeled as described above with paramagnetic ions, ions, radioactive isotopes, fluorescent dyes (e.g., fluorescein, rhodamine), enzymes and the like. It is understood that the choice of a reporter molecule will depend upon the detection system used.

Methods of Identifying Agents that can Inhibit Versican-Mediated Metastases

The invention further provides screening assays that are useful for generating or identifying therapeutic agents for prevention and treatment of metastatic cancer and assays for generating or identifying agents that inhibit versican-related metastasis. In particular, versican may be used in a variety of assays for generating metastasis and for identifying factors that inhibit such metastasis.

For example, in one embodiment, the invention relates to a method of identifying a therapeutic agent that can inhibit versican-mediated metastasis. Such a method can involve use of an animal model for metastatic cancer. For example, a method of identifying a therapeutic agent can involve administering a test agent to an experimental animal that expresses versican in myeloid progenitor cells and observing whether a primary tumor in the experimental animal metastasizes to a site distal from the primary tumor site. In some embodiments, the method also includes comparing the number of distal metastases compared to a control experimental animal that has also been administered the test agent but where the control experimental animal does not express versican.

Examples of experimental animals that can be employed include mice, rats, dogs, goats, monkeys, and chimpanzees. In general any experimental animal can be employed so long as it is susceptible to metastasis, particularly if the animal is susceptible to metastasis of human cancer cells that have been administered to the experimental animal. One type of mouse strain that can be used is the FVB.Cg-Tg(ACTB-EGFP)B5Nagy/J mouse strain (available from the Jackson Laboratory (Bar Harbor, Me.), which express green fluorescent protein (GFP) in bone marrow cells. Use of such a mouse strain allows bone marrow cells (e.g., myeloid progenitor cells that express versican) to be detected as they disperse from the bone marrow into other tissues.

Dosages of known and newly identified therapeutic agents can also be determined by use of such methods. For example, in one embodiment, the invention includes a method of identifying dosage of a therapeutic agent that can inhibit versican-mediated metastasis. Such a method can involve administering a series of test dosages of a therapeutic agent to an experimental animal that expresses versican in myeloid progenitor cells and observing which dosage(s) inhibit metastasis of a primary tumor in the experimental animal to a site distal from the primary tumor site.

The present invention also provides a method of evaluating a therapeutically effective dosage for treating a metastatic cancer with a versican inhibitor or a test agent that includes determining the LD100 or ED50 of the agent in vitro. Such a method permits calculation of the approximate amount of agent needed per volume to inhibit metastatic cancer cell growth or to kill 50% to 100% of the metastatic cancer cells. Such amounts can be determined, for example, by standard microdilution methods in cultured cells or by administration of varying amounts of a versican inhibitor or a test agent to an experimental animal.

Test agents and test dosages that can successfully inhibit versican-mediated metastasis can reduce the metastasis of a primary tumor by any amount such as, for example, by at least 2%, 5%, 10%, 20%, 40%, 50%, 60%, 70%, 80%, 90%, 95% or more than 95%. A therapeutically effective dosage is also one that is substantially non-toxic. For example, a therapeutically effective dosage is a dosage that does not adversely affect the production of differentiated cells from the bone marrow such as immune cells (e.g., T cells and/or B cells), erythrocytes, lymphocytes, or combinations thereof.

### Anti-Versican Antibodies

The invention provides antibody preparations directed against versican, for example, antibodies capable of binding a polypeptide having SEQ ID NO: 1 or SEQ ID NO:2. Such antibodies are desirable to detect versican in vitro or in vivo and for use in detecting metastatic cancer and metastatic tumors. Moreover, antibody preparations of the invention can serve as inhibitors of versican and therefore act as therapeutic agents.

Methods are provided to prepare and screen for antibodies that preferentially recognize versican. For example, versican can be used as antigen to raise polyclonal or monoclonal antibodies. The resultant antibodies can be selected for binding to a selected versican sequence, for binding to any versican sequence, for selectivity of binding to versican, for high affinity binding to versican and/or for inhibition of versican. Inhibitory antibodies can be selected by screening the antibodies for inhibition of cultured cancer cell growth and/or for inhibition versican-mediated metastasis, for example, using methods described herein.

Antibody molecules belong to a family of plasma proteins called immunoglobulins, whose basic building block, the immunoglobulin fold or domain, is used in various forms in many molecules of the immune system and other biological recognition systems. A typical immunoglobulin has four polypeptide chains, containing an antigen binding region known as a variable region and a non-varying region known as the constant region.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82, 4592-4596 (1985).

Depending on the amino acid sequences of the constant domain of their heavy chains, immunoglobulins can be assigned to different classes. There are at least five (5) major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular toxicity.

An antibody that is contemplated for use in the present invention thus can be in any of a variety of forms, including a whole immunoglobulin, an antibody fragment such as Fv. Fab, and similar fragments, a single chain antibody which includes the variable domain complementarity determining regions (CDR), and the like forms, all of which fall under the broad term “antibody”, as used herein. The present invention contemplates the use of any specificity of an antibody, polyclonal or monoclonal, and is not limited to antibodies that recognize and immunoreact with a specific antigen. In preferred embodiments, in the context of both the therapeutic and screening methods described below, an antibody or fragment thereof is used that is immunospecific for an antigen or epitope of the invention.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments that are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, refers to Fv, F(ab) and F(ab′)₂ fragments.

Antibody fragments contemplated by the invention are therefore not full-length antibodies but do have similar or improved immunological properties relative to an anti-versican antibody. Such antibody fragments may be as small as about 4 amino acids, 5 amino acids, 6 amino acids, 7 amino acids, 9 amino acids, about 12 amino acids, about 15 amino acids, about 17 amino acids, about 18 amino acids, about 20 amino acids, about 25 amino acids, about 30 amino acids or more. In general, an antibody fragment of the invention can have any upper size limit so long as it binds with specificity to versican, e.g. a polypeptide having SEQ ID NO: 1.

Antibody fragments retain some ability to selectively bind with its antigen. Some types of antibody fragments are defined as follows:

(1) Fab is the fragment that contains a monovalent antigen-binding fragment of an antibody molecule. A Fab fragment can be produced by digestion of whole antibody with the enzyme papain to yield an intact light chain and a portion of one heavy chain.

(2) Fab′ is the fragment of an antibody molecule can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

(3) (Fab′)₂ is the fragment of an antibody that can be obtained by treating whole antibody with the enzyme pepsin without subsequent reduction. F(ab′)₂ is a dimer of two Fab′ fragments held together by two disulfide bonds.

(4) Fv is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv including only three CDRs specific for an antigen) has the ability to recognize and bind antigen, although at a lower affinity than the entire binding site.

(5) Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further includes a polypeptide linker between the VH and VL domains that enables the sFv to form the desired structure for antigen binding. For a review of sFv see Pluckthun in The Pharmacology of Monoclonal Antibodies, vol. 113, Rosenburg and Moore eds. Springer-Verlag, N.Y., pp. 269-315 (1994).

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Hollinger et al., Proc. Natl. Acad. Sci. USA 90: 6444-6448 (1993).

Methods for preparing polyclonal antibodies are available to those skilled in the art. See, for example, Green, et al., Production of Polyclonal Antisera, in: Immunochemical Protocols (Manson, ed.), pages 1-5 (Humana Press); Coligan, et al., Production of Polyclonal Antisera in Rabbits, Rats Mice and Hamsters, in: Current Protocols in Immunology, section 2.4.1 (1992), which are hereby incorporated by reference.

Methods for preparing monoclonal antibodies are likewise available to one of skill in the art. See, for example, Kohler & Milstein, Nature, 256:495 (1975); Coligan, et al., sections 2.5.1-2.6.7; and Harlow, et al., in: Antibodies: A Laboratory Manual, page 726 (Cold Spring Harbor Pub. (1988)), which are hereby incorporated by reference. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan, et al., sections 2.7.1-2.7.12 and sections 2.9.1-2.9.3; Barnes, et al., Purification of Immunoglobulin G (IgG), in: Methods in Molecular Biology, Vol. 10, pages 79-104 (Humana Press (1992).

Methods of in vitro and in vivo manipulation of monoclonal antibodies are also available to those skilled in the art. For example, monoclonal antibodies to be used in accordance with the present invention may be made by the hybridoma method first described by Kohler and Milstein, Nature 256, 495 (1975), or may be made by recombinant methods, e.g., as described in U.S. Pat. No. 4,816,567. The monoclonal antibodies for use with the present invention may also be isolated from phage antibody libraries using the techniques described in Clackson et al. Nature 352: 624-628 (1991), as well as in Marks et al., J. Mol. Biol. 222: 581-597 (1991). Another method involves humanizing a monoclonal antibody by recombinant means to generate antibodies containing human specific and recognizable sequences. See, for review, Holmes, et al., J. Immunol., 158:2192-2201 (1997) and Vaswani, et al., Annals Allergy, Asthma & Immunol., 81:105-115 (1998).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional polyclonal antibody preparations that typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates that the antibody preparation is a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al. Proc. Natl. Acad. Sci. 81, 6851-6855 (1984).

Methods of making antibody fragments are also known in the art (see for example, Harlow and Lane, Antibodies: A Laboratory Manual, Cold Spring Harbor Laboratory, New York, (1988), incorporated herein by reference). Antibody fragments of the present invention can be prepared by proteolytic hydrolysis of the antibody or by expression in E. coli of DNA encoding the fragment. Antibody fragments can be obtained by pepsin or papain digestion of whole antibodies conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fe fragment directly. These methods are described, for example, in U.S. Pat. No. 4,036,945 and U.S. Pat. No. 4,331,647, and references contained therein. These patents are hereby incorporated in their entireties by reference.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. For example, Fv fragments comprise an association of V_{H} and V_{L} chains. This association may be non-covalent or the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow, et al., Methods: a Companion to Methods in Enzymology, Vol. 2, page 97 (1991); Bird, et al., Science 242:423-426 (1988); Ladner, et al, U.S. Pat. No. 4,946,778; and Pack, et al., Bio/Technology 11:1271-77 (1993).

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The invention contemplates human and humanized forms of non-human (e.g. murine) antibodies. Such humanized antibodies are chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody) such as mouse, rat or rabbit having the desired specificity, affinity and capacity.

In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are made to further refine and optimize antibody performance. In general, humanized antibodies will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc). typically that of a human immunoglobulin. For further details, see: Jones et al., Nature 321, 522-525 (1986); Reichmann et al., Nature 332, 323-329 (1988); Presta, Curr. Op. Struct. Biol. 2, 593-596 (1992); Holmes, et al., J. Immunol., 158:2192-2201 (1997) and Vaswani, et al., Annals Allergy, Asthma & Immunol., 81:105-115 (1998).

The invention also provides methods of mutating antibodies to optimize their affinity, selectivity, binding strength or other desirable property. A mutant antibody refers to an amino acid sequence variant of an antibody. In general, one or more of the amino acid residues in the mutant antibody is different from what is present in the reference antibody. Such mutant antibodies necessarily have less than 100% sequence identity or similarity with the reference amino acid sequence. In general, mutant antibodies have at least 75% amino acid sequence identity or similarity with the amino acid sequence of either the heavy or light chain variable domain of the reference antibody. Preferably, mutant antibodies have at least 80%, more preferably at least 85%, even more preferably at least 90%, and most preferably at least 95% amino acid sequence identity or similarity with the amino acid sequence of either the heavy or light chain variable domain of the reference antibody. One method of mutating antibodies involves affinity maturation using phage display.

The invention is therefore directed to a method for selecting antibodies and/or antibody fragments or antibody polypeptides with desirable properties. Such desirable properties can include increased binding affinity or selectivity for the epitopes of the invention

The antibodies and antibody fragments of the invention are isolated antibodies and antibody fragments. An isolated antibody is one that has been identified and separated and/or recovered from a component of the environment in which it was produced. Contaminant components of its production environment are materials that would interfere with diagnostic or therapeutic uses for the antibody, and may include enzymes, hormones, and other proteinaceous or nonproteinaceous solutes. The term “isolated antibody” also includes antibodies within recombinant cells because at least one component of the antibody's natural environment will not be present. Ordinarily, however, isolated antibody will be prepared by at least one purification step

If desired, the antibodies of the invention can be purified by any available procedure. For example, the antibodies can be affinity purified by binding an antibody preparation to a solid support to which the antigen used to raise the antibodies is bound. After washing off contaminants, the antibody can be eluted by known procedures. Those of skill in the art will know of various techniques common in the immunology arts for purification and/or concentration of polyclonal antibodies, as well as monoclonal antibodies (see for example, Coligan, et al., Unit 9, Current Protocols in Immunology, Wiley Interscience, 1991, incorporated by reference).

In some embodiments, the antibody will be purified as measurable by at least three different methods: 1) to greater than 95% by weight of antibody as determined by the Lowry method, and most preferably more than 99% by weight; 2) to a degree sufficient to obtain at least 15 residues of N-terminal or internal amino acid sequence by use of a spinning cup sequentator; or 3) to homogeneity by SDS-PAGE under reducing or non-reducing conditions using Coomasie blue or, preferably, silver stain.

### Compositions

The versican inhibitors and/or versican binding agents can be formulated as compositions with or without additional therapeutic agents, and administered to an animal, such as a human patient, in a variety of forms adapted to the chosen route of administration. Routes for administration include, for example, oral, local, parenteral, intraperitoneal, intravenous and intraarterial routes.

The compositions can be formulated as pharmaceutical dosage forms. Such pharmaceutical dosage forms can include (a) liquid solutions; (b) tablets, sachets, or capsules containing liquids, solids, granules, or gelatin; (c) suspensions in an appropriate liquid; and (d) suitable emulsions.

Solutions of the active agents (versican inhibitors, other therapeutic agents and/or versican binding agents) can be prepared in water or saline, and optionally mixed with other agents. For example, formulations for intravenous or intraarterial administration may include sterile aqueous solutions that may also contain buffers, diluents, stabilizing agents, nontoxic surfactants, chelating agents, polymers and/or other suitable additives. Sterile injectable solutions are prepared by incorporating the active agents in the required amount in the appropriate solvent with various of the other ingredients, in a sterile manner or followed by sterilization (e.g., filter sterilization) after assembly.

In another embodiment, active agent-lipid particles can be prepared and incorporated into a broad range of lipid-containing dosage forms. For instance, the suspension containing the active agent-lipid particles can be formulated and administered as liposomes, gels, oils, emulsions, topical creams, pastes, ointments, lotions, foams, mousses, and the like.

In some embodiments, the active agents may be formulated in liposome compositions. Sterile aqueous solutions, active agent-lipid particles or dispersions comprising the active agent(s) are adapted for administration by encapsulation in liposomes. Such liposomal formulations can include an effective amount of the liposomally packaged active agent(s) suspended in diluents such as water, saline, or PEG 400.

The liposomes may be unilamellar or multilamellar and are formed of constituents selected from phosphatidylcholine, dipalmitoylphosphatidylcholine, cholesterol, phosphatidylethanolamine, phosphatidylserine, demyristoylphosphatidylcholine and combinations thereof. The multilamellar liposomes comprise multilamellar vesicles of similar composition to unilamellar vesicles, but are prepared so as to result in a plurality of compartments in which the silver component in solution or emulsion is entrapped. Additionally, other adjuvants and modifiers may be included in the liposomal formulation such as polyethyleneglycol, or other materials.

While a suitable formulation of liposome includes dipalmitoyl-phosphatidylcholine:cholesterol (1:1) it is understood by those skilled in the art that any number of liposome bilayer compositions can be used in the composition of the present invention. Liposomes may be prepared by a variety of known methods such as those disclosed in U.S. Pat. No. 4,235,871 and in RRC, Liposomes: A Practical Approach. IRL Press, Oxford, 1990, pages 33-101.

The liposomes containing the active agents may have modifications such as having non-polymer molecules bound to the exterior of the liposome such as haptens, enzymes, antibodies or antibody fragments, cytokines and hormones and other small proteins, polypeptides or non-protein molecules which confer a desired enzymatic or surface recognition feature to the liposome. Surface molecules which preferentially target the liposome to specific organs or cell types include for example antibodies which target the liposomes to cells bearing specific antigens. Techniques for coupling such molecules are well known to those skilled in the art (see for example U.S. Pat. No. 4,762,915 the disclosure of which is incorporated herein by reference). Alternatively, or in conjunction, one skilled in the art would understand that any number of lipids bearing a positive or negative net charge may be used to alter the surface charge or surface charge density of the liposome membrane. The liposomes can also incorporate thermal sensitive or pH sensitive lipids as a component of the lipid bilayer to provide controlled degradation of the lipid vesicle membrane.

Liposome formulations for use with active agents may also be formulated as disclosed in WO 2005/105152 (the disclosure of which is incorporated herein in its entirety). Briefly, such formulations comprise phospholipids and steroids as the lipid component. These formulations help to target the molecules associated therewith to in vivo locations without the use of an antibody or other molecule.

Antibody-conjugated liposomes, termed immunoliposomes, can be used to carry active agent(s) within their aqueous compartments. Compositions of active agent(s) provided within antibody labeled liposomes (immunoliposomes) can specifically target the active agent(s) to a particular cell or tissue type to elicit a localized effect. Methods for making of such immunoliposomal compositions are available, for example, in Selvam M. P., et al., 1996. Antiviral Res. December; 33(1): 11-20 (the disclosure of which is incorporated herein in its entirety).

For example, immunoliposomes can specifically deliver active agents to the cells possessing a unique antigenic marker recognized by the antibody portion of the immunoliposome. Immunoliposomes are ideal for the in vivo delivery of active agent(s) to target tissues due to simplicity of manufacture and cell-specific specificity.

Tumor-specific antibodies can be used in conjunction with the inhibitors or liposomes containing inhibitors. Other active agents can also be included in such liposomes. Antibodies such as anti-CD11b antibodies, anti-CD33 antibodies, anti-VEGF receptor antibodies, anti-alphafetoprotein (AFP) antibodies, anti-carcinoembryonic antigen (CEA) antibodies, anti-CA-125 antibodies, anti-MUC-1 antibodies, anti-epithelial tumor antigen (ETA) antibodies, anti-tyrosinase antibodies, anti-ras antibodies, anti-p53 antibodies and antibodies directed against melanoma-associated antigen 1 (MAGE1) can be used in liposomes. For example, the antibodies can be mixed with or tethered to the lipids making up the liposomal shell. VEGF receptor is highly expressed in various tumor-related cells. The entire coding sequences for all MAGE genes are located within the last exon, which exhibits 64 to 85% homology with the sequence of MAGE1. Active agents including versican inhibitors can be loaded into liposomes following conjugation of liposomal lipids with antibodies that specifically bind CD11b, CD33, VEGF receptor, AFP, CEA, CA-125, MUC-1, ETA, tyrosinase, ras, p53, MAGE1, or combinations of antibodies directed against these or other tumor antigens.

In some instances, the active agents can be administered orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, they may be combined with one or more excipients and used in the form of ingestible tablets, buccal tablets, troches, capsules, elixirs, suspensions, syrups, wafers, and the like. Such compositions and preparations may contain at least 0.1% of active compound. The percentage of the compositions and preparations may, of course, be varied. The amount of compound in such therapeutically useful compositions is such that an effective dosage level will be obtained.

The active agents can also be incorporated into dosage forms such as tablets, troches, pills, and capsules. These dosage forms may also contain any of the following: binders such as gum tragacanth, acacia, corn starch or gelatin; excipients such as dicalcium phosphate; a disintegrating agent such as corn starch, potato starch, alginic acid and the like; a lubricant such as magnesium stearate; polymers such as cellulose-containing polymers (e.g., hydroxypropyl methylcellulose, methylcellulose, ethylcellulose), polyethylene glycol, poly-glutamic acid, poly-aspartic acid or poly-lysine; and a sweetening agent such as sucrose, fructose, lactose or aspartame or a flavoring agent such as peppermint, oil of wintergreen, or cherry flavoring may be added.

Tablet formulations can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active agents in a flavoring or sweetener, e.g., sucrose, as well as pastilles comprising the active agent(s) in an inert base, such as gelatin and glycerin or sucrose and acacia emulsions, gels, and the like containing carriers known in the art.

When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In some embodiments, one or more of the active agents are linked to polyethylene glycol (PEG). For example, one of skill in the art may choose to link an active agent to PEG to form the following pegylated drug.

Useful dosages of the active agents (e.g., versican inhibitors) can be determined by comparing their in vitro activity, and in vivo activity in animal models, for example, as described herein. Methods for the extrapolation of effective dosages in mice, and other animals, to humans are known to the art; for example, see U.S. Pat. No. 4,938,949. The compound can be conveniently administered in unit dosage form.

The desired dose may conveniently be presented in a single dose or as divided doses administered at appropriate intervals, for example, as two, three, four or more sub-doses per day. The sub-dose itself may be further divided, for example, into a number of discrete loosely spaced administrations; such as multiple oral, intraperitoneal or intravenous doses. For example, it can be desirable to administer the present compositions intravenously over an extended period, either by continuous infusion or in separate doses.

The therapeutically effective amount of the active agent(s) (e.g., a versican inhibitor) necessarily varies with the subject and the disease or physiological problem to be treated. As one skilled in the art would recognize, the amount can be varied depending on the method of administration. The amount of the active agent (e.g., inhibitor) for use in treatment will vary not only with the route of administration, but also the nature of the condition being treated and the age and condition of the patient and will be ultimately at the discretion of the attendant physician or clinician.

The pharmaceutical compositions of the invention can include an effective amount of at least one of the active agents of the invention (e.g., versican inhibitors), or two or more different agents of the invention (e.g., two or more versican inhibitors). These compositions can also include a pharmaceutically effective carrier.

The pharmaceutical compositions of the invention can also include other active ingredients and therapeutic agents, for example, other chemotherapeutic agents, anti-inflammatory agents, analgesics, vitamins, and the like. It is also within the scope of the present invention to combine any of the methods and any of the compositions disclosed herein with conventional cancer therapies, anti-cancer agents and various drugs in order to enhance the efficacy of such methods and/or compositions. For example, methods and compositions containing combinations of active agents can act through different mechanisms to improve the efficacy or speed of treatment. Methods and compositions containing combinations of active agents can also reduce the doses/toxicity of conventional therapies and/or to increase the sensitivity of conventional therapies.

One conventional therapy that can be used in conjunction with the methods and compositions containing combinations of active agents is surgery to remove primary and/or identified sites of metastatic tumors. Other conventional therapies that can be employed include radiation therapy or other types of chemotherapeutic drugs. Chemotherapeutic drugs that can be used include anti-cancer drugs known in the art, including but not limited to any radioactive drug, topoisomerase inhibitor, DNA binding agent, anti-metabolite, cytoskeletal-interacting drugs, ionizing radiation, or a combination of two or more of such known DNA damaging agents.

Cytoskeletal drugs are small molecules that interact with actin or tubulin. Any such cytoskeletal drug can be used in the methods and compositions described herein. Cytoskeletal drugs include paclitaxel, colchicine, cytochalasins, demecolcine, latsunculin, nocodazole, phalloidin, swinholide and vinblastine. Some cytoskeletal drugs stabilize a cytoskeletal component, for example, paclitaxel stabilizes microtubules. Other cytoskeletal drugs prevent polymerization. For example, cytochalasin D binds to actin monomers and prevents polymerization of actin filaments. In some embodiments, the anti-cancer agent is paclitaxel.

A topoisomerase inhibitor that can be used in conjunction with the invention can be, for example, a topoisomerase I (Topo I) inhibitor, a topoisomerase II (Topo II) inhibitor, or a dual topoisomerase I and II inhibitor. A topo I inhibitor can be from any of the following classes of compounds: camptothecin analogue (e.g., karenitecin, aminocamptothecin, lurtotecan, topotecan, irinotecan, BAY 56-3722, rubitecan, GI14721, exatecan mesylate), rebeccamycin analogue, PNU 166148, rebeccamycin, TAS-103, camptothecin (e.g., camptothecin polyglutamate, camptothecin sodium), intoplicine, ecteinascidin 743, J-107088, pibenzimol. Examples of preferred topo I inhibitors include but are not limited to camptothecin, topotecan (hycaptamine), irinotecan (irinotecan hydrochloride), belotecan, or an analogue or derivative thereof.

A topo II inhibitor that can be used in conjunction with the invention can be, for example, from any of the following classes of compounds: anthracycline antibiotics (e.g., carubicin, pirarubicin, daunorubicin citrate liposomal, daunomycin, 4-iodo-4-doxydoxorubicin, doxorubicin, docetaxel, n,n-dibenzyl daunomycin, morpholinodoxorubicin, aclacinomycin antibiotics, duborimycin, menogaril, nogalamycin, zorubicin, epirubicin, marcellomycin, detorubicin, annamycin, 7-cyanoquinocarcinol, deoxydoxorubicin, idarubicin, GPX-100, MEN-10755, valrubicin, KRN5500), epipodophyllotoxin compound (e.g., podophyllin, teniposide, etoposide, GL331, 2-ethylhydrazide), anthraquinone compound (e.g., ametantrone, bisantrene, mitoxantrone, anthraquinone), ciprofloxacin, acridine carboxamide, amonafide, anthrapyrazole antibiotics (e.g., teloxantrone, sedoxantrone trihydrochloride, piroxantrone, anthrapyrazole, losoxantrone), TAS-103, fostriecin, razoxane, XK469R, XK469, chloroquinoxaline sulfonamide, merbarone, intoplicine, elsamitrucin, CI-921, pyrazoloacridine, elliptinium, amsacrine. Examples of preferred topo II inhibitors include but are not limited to doxorubicin (Adriamycin), etoposide phosphate (etopofos), teniposide, sobuzoxane, or an analogue or derivative thereof.

DNA binding agents that can be used in conjunction with the invention include but are not limited to DNA groove binding agent, e.g., DNA minor groove binding agent; DNA crosslinking agent; intercalating agent; and DNA adduct forming agent. A DNA minor groove binding agent can be an anthracycline antibiotic, mitomycin antibiotic (e.g., porfiromycin, KW-2149, mitomycin B, mitomycin A, mitomycin C), chromomycin A3, carzelesin, actinomycin antibiotic (e.g., cactinomycin, dactinomycin, actinomycin F1), brostallicin, echinomycin, bizelesin, duocarmycin antibiotic (e.g., KW 2189), adozelesin, olivomycin antibiotic, plicamycin, zinostatin, distamycin, MS-247, ecteinascidin 743, amsacrine, anthramycin, and pibenzimol, or an analogue or derivative thereof.

DNA crosslinking agents include but are not limited to antineoplastic alkylating agent, methoxsalen, mitomycin antibiotic, psoralen. An antineoplastic alkylating agent can be a nitrosourea compound (e.g., cystemustine, tauromustine, semustine, PCNU, streptozocin, SarCNU, CGP-6809, carmustine, fotemustine, methylnitrosourea, nimustine, ranimustine, ethylnitrosourea, lomustine, chlorozotocin), mustard agent (e.g., nitrogen mustard compound, such as spiromustine, trofosfamide, chlorambucil, estramustine, 2,2,2-trichlorotriethylamine, prednimustine, novembichin, phenamet, glufosfamide, peptichemio, ifosfamide, defosfamide, nitrogen mustard, phenesterin, mannomustine, cyclophosphamide, melphalan, perfosfamide, mechlorethamine oxide hydrochloride, uracil mustard, bestrabucil, DHEA mustard, tallimustine, mafosfamide, aniline mustard, chlomaphazine; sulfur mustard compound, such as bischloroethylsulfide; mustard prodrug, such as TLK286 and ZD2767), ethylenimine compound (e.g., mitomycin antibiotic, ethylenimine, uredepa, thiotepa, diaziquone, hexamethylene bisacetamide, pentamethylmelamine, altretamine, carzinophilin, triaziquone, meturedepa, benzodepa, carboquone), alkylsulfonate compound (e.g., dimethylbusulfan, Yoshi-864, improsulfan, piposulfan, treosulfan, busulfan, hepsulfam), epoxide compound (e.g., anaxirone, mitolactol, dianhydrogalactitol, teroxirone), miscellaneous alkylating agent (e.g., ipomeanol, carzelesin, methylene dimethane sulfonate, mitobronitol, bizelesin, adozelesin, piperazinedione, VNP40101M, asaley, 6-hydroxymethylacylfulvene, EO9, etoglucid, ecteinascidin 743, pipobroman), platinum compound (e.g., ZD0473, liposomal-cisplatin analogue, satraplatin, BBR 3464, spiroplatin, ormaplatin, cisplatin, oxaliplatin, carboplatin, lobaplatin, zeniplatin, iproplatin), triazene compound (e.g., imidazole mustard, CB 10-277, mitozolomide, temozolomide, procarbazine, dacarbazine), picoline compound (e.g., penclomedine), or an analogue or derivative thereof. Examples of preferred alkylating agents include but are not limited to cisplatin, dibromodulcitol, fotemustine, ifosfamide (ifosfamid), ranimustine (ranomustine), nedaplatin (latoplatin), bendamustine (bendamustine hydrochloride), eptaplatin, temozolomide (methazolastone), carboplatin, altretamine (hexamethylmelamine), prednimustine, oxaliplatin (oxalaplatinum), carmustine, thiotepa, leusulfon (busulfan), lobaplatin, cyclophosphamide, bisulfan, melphalan, and chlorambucil, or analogues or derivatives thereof.

Intercalating agents can be an anthraquinone compound, bleomycin antibiotic, rebeccamycin analogue, acridine, acridine carboxamide, amonafide, rebeccamycin, anthrapyrazole antibiotic, echinomycin, psoralen, LU 79553, BW A773U, crisnatol mesylate, benzo(a)pyrene-7,8-diol-9,10-epoxide, acodazole, elliptinium, pixantrone, or an analogue or derivative thereof, etc.

DNA adduct forming agents include but are not limited to enediyne antitumor antibiotic (e.g., dynemicin A, esperamicin Al, zinostatin, dynemicin, calicheamicin gamma 1I), platinum compound, carmustine, tamoxifen (e.g., 4-hydroxy-tamoxifen), psoralen, pyrazine diazohydroxide, benzo(a)pyrene-7,8-diol-9,10-epoxide, or an analogue or derivative thereof.

Anti-metabolites include but are not limited to cytosine, arabinoside, floxuridine, fluorouracil, mercaptopurine, Gemcitabine, and methotrexate (MTX).

Monoclonal antibodies, cancer vaccines, angiogenesis inhibitors, and gene therapy are targeted therapies that can also be combined into the versican inhibitor compositions and used in the methods described herein.

The ultimate dosage form should be sterile, fluid and stable under the conditions of manufacture and storage.

### Kits

Another aspect of the invention is one or more kits for determining and/or treating the metastatic status of a test tissue sample.

In one embodiment, the kits of the present invention comprise one or more probes and/or primers each capable of specifically binding to a sequence of at least 15, 20, 25, 30, 40, 50 nucleotides, or any number of nucleotides between 13-50, in a versican RNA. The probes may be part of an array. Alternatively, the probes or primers may be packaged separately and/or individually. In some embodiments, the probes or primers may be detectably labeled. The versican mRNA can have a sequence such as SEQ ID NO: 3, a selected fragment thereof. The versican mRNA can also have a sequence with at least 90% sequence identity to SEQ ID NO: 3, a selected fragment thereof.

In another embodiment, the kits of the present invention comprise one or more antibodies and/or antibody fragments each capable of specifically binding to a versican polypeptide.

Additional reagents can be included in the kits. For example, the kits may also contain reagents for determining the expression levels of versican in a test tissue sample. Such reagents can include reagents for isolating, storing and detecting a versican RNA or versican polypeptide within a test sample. Such reagents can include reagents for isolating, storing and detecting a tissue sample that may contain a versican mRNA or polypeptide.

For example, the kits can include reagents and enzymes for nucleic acid amplification and/or for reverse transcription of a versican mRNA. The kits may also include reagents such as solutions stabilizing RNA, solutions for purifying RNA, buffers, or other reagents that can be used in obtaining the expression levels of mRNAs in a test tissue sample. Prevention of the action of microorganisms can be ensured by the inclusion of various antibacterial and antifungal agents, for example, paraben, chlorobutanol, phenol sorbic acid, and the like. It may also be desirable to include agents such as solvents for RNA, reducing agents (e.g., beta-mercaptoethanol), RNA stabilizing reagents (e.g., reagents for inhibiting ribonucleases, disrupting tissues, precipitating RNA, and the like).

In other embodiments, the kits can include a container or system that includes at least one primary antibody that can bind to a versican polypeptide. Such an antibody can be attached or adsorbed onto a solid surface. A secondary antibody can also be provided in a separate container for detection of a complex between the at least one primary antibody and a versican polypeptide. The primary or secondary antibody can be linked to a label.

In another embodiment, the kits can include a container or system that includes at least one versican binding agent. Such a versican binding agent can also be attached or adsorbed onto a solid surface. Such a versican binding agent can be linked to a label. In some embodiments, the kit can include an antibody for detection of a complex between the a versican binding agent and a versican polypeptide.

The kits can also include a versican inhibitor, a chemotherapeutic agent or anti-cancer agent, for example, any of the versican inhibitors, chemotherapeutic agents or anti-cancer agents described herein.

The following non-limiting Examples illustrate materials and methods used for development of the invention.

### Example 1

### Materials and Methods

This Example illustrates some of the procedures employed in the development of the invention.

### Mice

FVB/n, FVB.Cg-Tg(ACTB-EGFP) B5Nagy/J, and MMTV-PyVT mice were obtained from The Jackson Laboratory (Bar Harbor, Me.). The FVB.Cg-Tg(ACTB-EGFP)B5Nagy/J mice express green fluorescent protein (GFP) in bone marrow cells. Male MMTV-PyMT transgenic mice were bred with wild-type FVB/N females. Female offspring (3 weeks) were genotyped to identify mice carrying the PyMT transgene. The positive mice spontaneously develop mammary tumors by 6 to 7 weeks of age and pulmonary metastases by 10 to 12 weeks of age. To quantify lung metastases in MMTV-PyMT mice, serial lung sections (at least 10) were prepared and stained with hematoxylin and eosin. Within the stained sections, areas depicting metastatic lesions and total lung were measured with ImageJ software.

CB-17 SCID mice were obtained from Charles River (Wilmington, Mass.).

All animal works were conducted in accordance with a protocol approved by the Institutional Animal Care and Use Committee at Weill Cornell Medical College.

### Cell Lines

The human breast cancer cell line (MDA-MB-231) was obtained from the American Type Culture Collection (ATCC). Cultures were resuscitated from stocks frozen at low passage within 6 months of purchase. Cell authentication was conducted at ATCC by short tandem repeat profiling, cell morphology monitoring, karyotyping, and the ATCC cytochrome e oxidase I (COI) assays. The morphology and metastatic behavior of MDA-MB-231 cells were tested by the inventors and by colleagues in the medical school. Cells were cultured in Dulbecco's Modified Eagle's Media with 10% FBS, 5 mmol/L glutamine, and 1% penicillin/streptomycin.

Versican-expressing cells (MDA-Vcn) were generated by transfection with a construct carrying the secreted form of human versican cDNA (pSecTag-V1) encoding versican with SEQ ID NO:1. Stably transfected cells were obtained by selecting cells with Zeocin (200 mg/mL). MDA-Cont cells were obtained by transfection of MDA-MB-231 cells with control empty vector and selected through the same procedure.

MDA-MB-231 cells were also labelled with luciferase-RFP (red fluorescent protein) fusion protein. Successfully transduced cells were sorted with FACS (Aria II, BD Bioscience) and expended for bioluminescent imaging in vivo.

### Human Samples

Human normal lung tissues (n=5) were obtained from ILSbio LLC (Chestertown, Md.). Human lungs bearing metastases from breast cancer patients were obtained from CT Surgery Department, Weill Cornell Medical College (New York), consented according to approved IRB protocols from the institution. Metastasis tissues from the lungs and livers of breast cancer patients (n=11) were obtained in the form of a tissue microarray from John Hopkins University School of Medicine, Baltimore, Md.

### Mouse Pulmonary Metastasis Models

MMTV-PyMT Model:

Male MMTV-PyMT transgenic mice in FVB/N background were bred with wild type FVB/N females. The female offspring (3 weeks) were genotyped to identify mice carrying the PyMT transgene. The positive mice spontaneously develop mammary tumors by 6-7 weeks of age and pulmonary metastases by 10-12 weeks of age.

MDA-MB-231 Model:

To generate experimental pulmonary metastasis model, 1×10⁶ viable MDA-MB-231, MDA-Cont or MDA-Vcn cells were injected into CB-17 SCID mice via tail vein in a volume of 0.1 mL. Pulmonary metastasis formation was monitored with in vivo bioluminescent imaging once per weeks. Four weeks after injection, lungs were harvested for histological analysis.

To harvest lungs, tumor-bearing mice were anesthetized, the chest was opened and perfusion was performed via the right ventricle with PBS followed by 4% paraformaldehyde. Lungs were fixed in 4% paraformaldehyde overnight and imaged under fluorescent steromicroscope (Nikon). Pulmonary metastases were counted under RFP channel and measured with the OptiONE software (Nikon). To quantify lung metastasis in MMTV-PyMT mice, serial lung sections (at least 10) were prepared and stained with Hematoxylin & Eosin (HE). The stained sections were scanned and areas depicting metastatic lesions and total lung were measured with ImageJ software.

### Bone Marrow Isolation, Lin⁻ Cell Purification and Transplantation

Bone marrow (BM) cells were harvested by flushing femurs and tibias of donor animals. BM transplantation was performed by injecting 1×10⁷ total BM cells via tail vein into lethally irradiated (900 rads) recipients as described (Gao et al., Science 319:195-198 (2008)).

In some experiments Lin⁻ cells were enriched using the Lineage Cell Depletion Kit (CD5, CD45R (B220), CD11b, anti-Gr-1, 7-4, and Ter 19 antibodies), and a magnetic separation device, MACS (Milteyni Biotech, Auburn, Calif.). In this case, the bone marrow transplantation was performed by using 5×10⁵ Lin⁻ cells per mouse.

### Short Hairpin RNA Design, Lentiviral Constructs, Virus Generation and Transductions

To suppress versican expression, mir30-based short hairpin RNAs (shRNAs) targeting mouse versican (V0/V1 isoform) were designed, and cloned into the XhoI/EcoRI site of the lentiviral construct pGIPZ (OpenBiosystem). Multiple hairpin constructs were screened for effective knockdown of versican. The 22mer targeting sequence that resulted in efficient knockdown included 5′-ACACCAGAATTAG AAAGTTCAA-3′ (shVcn1; SEQ ID NO:43) and 5′-AGCACCTTGTCTGATGGCCAAG-3′ (shVcn2; SEQ ID NO:44) shRNA. Targeting firefly luciferase served as a non-specific control.

Lentivirus was generated and concentrated using available procedures. Lentiviral transductions of Lin⁻ bone marrow cells were conducted as described by Gao et al., Science 319:195-198 (2008)). For example, pGIPZ constructs were co-transfected with packaging constructs pMD2G and psPAX2 into 293T cells. Culture medium containing lentivirus was collected at 48 and 72 h after transfection and concentrated approximately 100-fold by ultracentrifugation. The titer of lentivirus was determined by infection of 293T cells and FACS analysis. Lentiviral transductions of Lin− BM cells were generally performed in serum-free StemSpan™ SFEM media (Stem Cell Technologies), in the presence of cytokines (IL3 20 ng/ml; IL6 100 ng/ml; SCF 100 ng/ml) for 12 h. Transduction efficiencies of >90% were obtained, as determined by analysis of GFP expression by flow cytometry.

### Conditioned Medium Experiments

To investigate effects of exogenous versican on MET of tumor cells, CD11b⁺Gr1⁺ cells were obtained from MMTV-PyMT mice (10-weeks old) by flow sorting. Sorted CD11b⁺Gr1⁻ cells were cultured in RPMI with 10% FBS using a density of 100,000 cells/mL in 6-well plates. Conditioned medium was harvested after 2 days.

MDAMB-231 cells were treated with the conditioned medium (1:1 dilution with fresh growth medium), or purified versican (25 μg/mL) or untreated for 3 days. Cells were then applied in cell proliferation assays or harvested for RT-PCR analysis.

### Versican Purification

For versican purification, 293T cells were transfected with a construct carrying a 6×His-tagged human versican (V1 isoform) nucleic acid segment. Supernatant was harvested 5 days after transfection. Versican was purified with Ni-NTA Fast Start Kit (Qiagen Inc). For cell proliferation assay, cells were treated with the conditioned medium, purified versican (2.5 mg/mL), or normal growth medium for 3 days. EdUrd (5-ethynyl-20-deoxyuridine; 10 nmol/L) was administered to culture medium for 30 minutes. The incorporation of EdUrd was detected using the Click-iT EdUrd Cell Proliferation Assay Kit (Invitrogen Inc.) and analysed by flow cytometry.

### Cell Proliferation, Apoptosis and Migration Assays

MDA-Cont and MDA-Vcn cells (1×10⁶) were cultured separately in suspension in non-adhesive 6-well plate (Corning Inc) for 1 day. For cell proliferation assay, EdU (10 nM) was administrated to culture medium to label proliferating cells for 30 min.

Cells were harvested, washed once with PBS and fixed with 4% paraformaldehyde for the 15 min at RT. Fixed cells were permeabilized and stained for EdU incorporation and DNA contains with Click-iT® EdU Cell Proliferation Assay kit (Invitrogen Inc) according to the standard protocol. Flow analysis of cell phases was performed with LSRII coupled with Diva software (BD Bioscience).

For cell apoptosis assay, the cells cultured in suspension were stained with Annexcin V-FITC (Annexin V:FITC apoptosis detection kit II, BD) according to the standard protocol and analysed by flow cytometry.

For cell migration assays, MDA-Cont and MDA-Vcn cells (2×10⁵) were seeded in a 6-well plate (Corning Inc) for 1 day. Cell migration videos were generated by obtaining images every 2 min for 2 hours under a computerized Zeiss microscope (Axio Observer) equipped with culture chamber. Cell moving was tracked and analysed with ImageJ and Manual Tracking Plugin software (NIH).

### Immunofluorescence and Microscopy

For immunofluorescence staining, tissues were fixed overnight in paraformaldehyde, followed by 20% sucrose, and cryoembedded in Tissue-tek O.C.T. embedding compound (Electron Microscopy Sciences). Sections (30 μm), were made and stained with the following primary antibodies, CD11b (clone ICRF44.), CD34 (clone 8G12), CD33 (Cat#555459, BD), Gr1 (Clone RB6-8C5), EpCam (Cat#347198, BD), Versican (Cat#V5639, Sigma and Clone 2B1 Seikagaku), E-cadherin (clone DECMA-1), Vimentin (Cat#550513, BD), and/or PyMT (NB 100-2749, Novus) using available procedures. Typically primary antibodies were directly conjugated to various Alexa Fluor dyes (Alexa 488, Alexa 568, and Alex 647) using antibody labelling kits (Invitrogen) performed as per manufacturer's instructions and purified over BioSpin P30 Gel (Biorad). GFP and RFP positive cells were detected by their intrinsic signal.

Fluorescent images were obtained using a computerized Zeiss fluorescent microscope (Axiovert 200M), fitted with an apotome and a HRM camera. Images were analysed by using Axiovision 4.6 software (Carl Zeiss Inc.).

### Immunohistochemical Staining

Paraffin embedded sections (5 μm) were dewaxed following standard protocol. Anti-versican (Cat#V5639, Sigma) or anti-CD11b (Cat#2729-1, Epitomics) antibodies were used for staining. For versican staining, sections were first treated with chondroitinase ABC (Sigma) overnight and then incubated with anti-versican antibody (Cat#V5639. Sigma). The antibody labelling was visualized by using DAKO Envision™ system (DAKO). For dual color staining, EnVision™ DuoFLEX Doublestain System was used to visualize CD11b and versican staining in brown and red color respectively. Images were taken with Olympus BX51 microscope coupled with Qcapture software (Olympus).

### Bioluminescent Imaging and Analysis

Mice inoculated with firefly luciferase expressing MDA-Cont or MDA-Vcn cells were anaesthetized and injected retro-orbitally with 75 mg/kg of D-luciferin (30 mg/mL in PBS). Imaging was taken with mice in a supine position between 2 and 5 min after injection with a Xenogen IVIS system coupled to Living Image acquisition and analysis software (Xenogen). For BLI plots, photon flux was calculated for each mouse by using a same rectangular region of interest encompassing the thorax of the mouse. BLI value was normalized to the value obtained immediately after xenografting (day 0), so that all mice had an arbitrary starting BLI signal of 100.

### Flow Cytometry and Cell Sorting

For analyses of the metastatic lungs or primary tumors, tissues were minced and then digested at 37° C. for 30-60 min with an enzyme cocktail (Collagenase A, elastase, and DNase I, Roche Applied Science). Digestion and incubation conditions were optimized to recover maximum cells with the highest viability as determined by trypan blue exclusion. Single cell suspensions were prepared by filtering through a 30-1 μm or 40-μm strainer. For analyses of the peripheral blood, blood was collected by cross-cut the tails of animals in anti-coagulant buffer (PBS with 5 mM EDTA). Red blood cells were eliminated by incubation in 1×Lysis Buffer (BD Bioscience) for 10 min at RT.

Cell suspensions were pre-blocked with 2% FBS plus Fc block (CD 16/CD32, 1:30, BD Biosciences PharMingen) and then incubated with the following primary antibodies from Pharmingen: rat IgG2ακ and IgG2αβ isotype control, CD11b (clone M1/70.), Gr1 (Clone RB6-8C5), Ly6C (Clone HK1.4), Ly6G (Clone 1A8), CD45 (Clone 30-F11), B200 (Clone RA3-6B2), CD3 (Clone 17A2), CD31 (Clone 390), PDGFR (Clone APA5), VEGFR1 (Clone MF1). SYTOX Blue (Invitrogen) was added in each cell staining tube to facilitate the elimination of dead cells in flow cytometry analysis. Single color stainings were freshly set up with selected antibodies and CompBeads (BD Bioscience) in each experiment for proper calibration of compensations.

Labelled cell populations were measured by LSRII flow cytometer coupled with FACS Diva software (BD Bioscience). Flow cytometry analysis was performed using a variety of controls including isotype antibodies, and FMO samples. For sorting, targeted cell populations were gated within FACS Diva software and sorted by Aria II sorter (BD Bioscience).

### Gene Expression Profiling

Single cell suspensions were prepared from metastatic lungs and control wild type lungs and stained with antibodies against CD11b and Gr1. Approximately 1×10⁵ CD11b⁺GR1⁺ myeloid progenitor cells were sorted by FACS (Aria II, BD Bioscience). Total RNA was extracted from the sorted cells using Picopure RNA Isolation kit (Arcturus). RNA quality was measured using Agilent's RNA 6000 Pico LabChip. Complementary DNA was synthesized and amplified with WT-Ovation™ Pico RNA Amplification System (NuGene Technologies Inc), followed by sense transcript cDNA generation, fragmentation and labelling with WT-Ovation™ Exon Module and FL-Ovation™ cDNA Biotin Module V2 (NuGene Technologies Inc).

Biotinylated cDNA was hybridized to GeneChip Mouse Exon 1.0 ST Array carrying probes representing 16,000 genes (Affymetrix, Santa Clara, Calif.) for gene expression analysis. Three biological replicates were performed in both tumor challenged and control groups. Statistical data analysis was performed using Array Assist (Stratagene) and Affymetrix Expression Console™ software.

### Quantitative RT-PCR Analysis

Total RNA was extracted with RNeasy kit (Qiagen) and converted to cDNA using Superscript II reverse transcriptase (Invitrogen). Q-PCR was performed with according primers and iQTM SYBER Green master mix (Biorad, Hercule, Calif.). Each sample was duplicated to minimize pipetting error. A standard protocol of initial denaturing at 95° C. for 3 min, 40 cycles of 95° C. for 20 sec, 60° C. for 30 sec, and 72° C. for 30 sec, followed by final extension at 72° C. for 5 min and melt curve analysis was applied on a BioRad CFX96 Real Time System (BioRad) coupled with Bio-Rad-CFX Manager software. The relative abundance of each transcript compared with the control was calculated.

Primer sequences for gene expression analysis include the following:

Mus-GAPDH-for:

(SEQ ID NO: 46)

GGTCCTCAGTGTAGCCCAAG;
Mus-GAPDH-rev:

(SEQ ID NO: 47)

AATGTGTCCGTCGTGGATCT;
Mus-Versican-for:

(SEQ ID NO: 48)

TGGGGTGAGAACCCTGTATCGTTT;
Mus-Versican-rev:

(SEQ ID NO: 49)

CCCATTGATATACTGCACTGACGG;
Mus-IL4-for:

(SEQ ID NO: 50)

GAAAACTCCATGCTTGAAGAAG;
Mus-IL4-rev:

(SEQ ID NO: 51)

TGATGTGGACTTGGACTCATTC;
Mus-IL10-for:

(SEQ ID NO: 52)

CCCCTGTGAAAATAAGAGCAAG;
Mus-IL10-rev:

(SEQ ID NO: 53)

GCTTCTATGCAGTTGATGAAGATG;
Mus-IL1b-for:

(SEQ ID NO: 54)

TGTGTAATGAAAGACGGCACAC;
Mus-IL1b-rev:

(SEQ ID NO: 55)

TCAAACTCCACTTTGCTCTTGA;
Mus-IL6-for:

(SEQ ID NO: 56)

AGACAAAGCCAGAGTCCTTCAG;
Mus-IL6-rev:

(SEQ ID NO: 57)

TTAGGAGAGCATTGGAAATTGG;
Mus-TNFa-for:

(SEQ ID NO: 58)

GTCTACTGAACTTCGGGGTGAT;
Mus-TNFa-rev:

(SEQ ID NO: 59)

TTGAGAAGATGATCTGAGTGTGAG;
MUS-ARG1-FOR:

(SEQ ID NO: 60)

CAGCTACCTGCTGGGAAGGAAGAA;
MUS-ARG1-REV:

(SEQ ID NO: 61)

CCAAGAGTTGGGTTCACTTCCA;
MUS-ARG2-FOR:

(SEQ ID NO: 62)

AGCAACCCTGTATTATGTATTTCT;
MUS-ARG2-REV:

(SEQ ID NO: 63)

ATCGACTTGGGATCCAGAAGGTGA;
MUS-NOS2-FOR:

(SEQ ID NO: 64)

ACCTACCGCACCCGAGATGGTCAG;
MUS-NOS2-REV:

(SEQ ID NO: 65)

CTGCTGCCAGAAACTTCGGAAGGG;
Hu-GAPDH-for:

(SEQ ID NO: 66)

CTTCAACAGCGACACCCACTCCTC;
Hu-GAPDH-rev:

(SEQ ID NO: 67)

GTCCACCACCCTGTTGCTGTAG;
Hu-Versican-for:

(SEQ ID NO: 68)

GAGTCAGTGGAAGGCACGGCAATC;
Hu-Versican-rev:

(SEQ ID NO: 69)

TGGTCTCCGCTGTATCCTGGCAC;
Hu-Ecadherin-for:

(SEQ ID NO: 70)

TGCCCAGAAAATGAAAAAGG;
Hu-Ecadherin-rev:

(SEQ ID NO: 71)

GTGTATGTGGCAATGCGTTC;
Hu-Snail-for:

(SEQ ID NO: 72)

CCTCCCTGTCAGATGAGGAC;
Hu-Snail-rev:

(SEQ ID NO: 73)

CCAGGCTGAGGTATTCCTTG;
Hu-vimentin-for:

(SEQ ID NO: 74)

TCTACGAGGAGGAGATGCGG;
Hu-vimentin-rev:

(SEQ ID NO: 75)

GGTCAAGACGTGCCAGAGAC;
Hu-Occludin-for:

(SEQ ID NO: 76)

TCCAATGGCAAAGTGAATGAC
and
Hu-Occludin-rev:

(SEQ ID NO: 77)

CTTTGCAGGTGCTCTTTTTGA.

### Western Blot Analysis

Cells were homogenized in 1×lysis buffer (BioRad) containing protease inhibitors (Roche Applied Science). Samples were boiled in 1×SDS sampling buffer, and loaded onto 4-20% gradient Tris-HCl gels (BioRad). Western blotting was performed by using antibodies specific for Versican (Cat#V5639, Sigma), E-cadherin (clone DECMA-1), Vimentin (Cat#550513, BD), Occludin (Cat#33-1500, Zymed) and β-actin (Sigma-Aldrich). For versican analysis, protein lysate were first dialyzed against chondroitinase buffer (40 mM Tris-acetate, pH=8.0, with protease inhibitors) and treated with chondroitinase ABC (Sigma) overnight.

### Statistical Analysis

Results were expressed as mean±SD. Analyses of different treatment groups were performed using the Student T test using the GraphPad Prism statistical program. P values <0.05 were considered significant. Error bars depict SD, except as otherwise indicated.

### Example 2

### Bone Marrow-Derived CD11b⁺Gr1⁺ Myeloid Cells are Recruited into Metastatic Lungs

To determine the contribution of the bone marrow-derived cells to the metastatic lung, syngeneic GFP⁺ bone marrow was transplanted into MMTV-PyMT transgenic mice (Guy et al., Mol Cell Biol 12:954-61 (1992)) using procedures described by Gao et al., Science 319:195-198 (2008); Nolan et al., Genes Dev 21:1546-58 (2007)). At about 6-7 weeks of age, spontaneous breast tumors metastasize to the lungs of these animals. By about 11-12 weeks of age the metastasized breast tumor cells form micrometastases and by week 16 macrometastases form (Gao et al., Science 319:195-198 (2008); Nolan et al., Genes Dev 21:1546-58 (2007)).

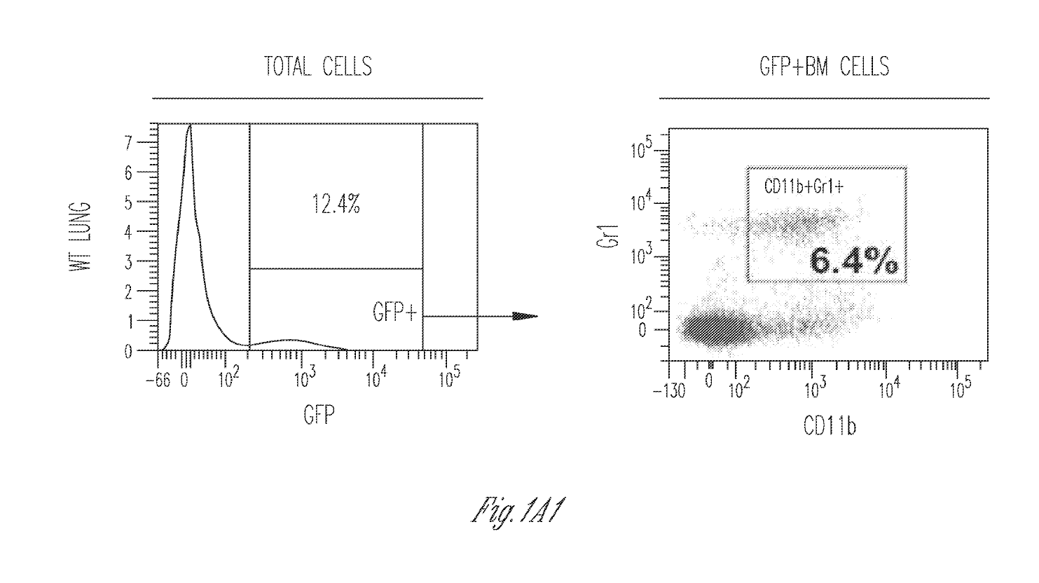

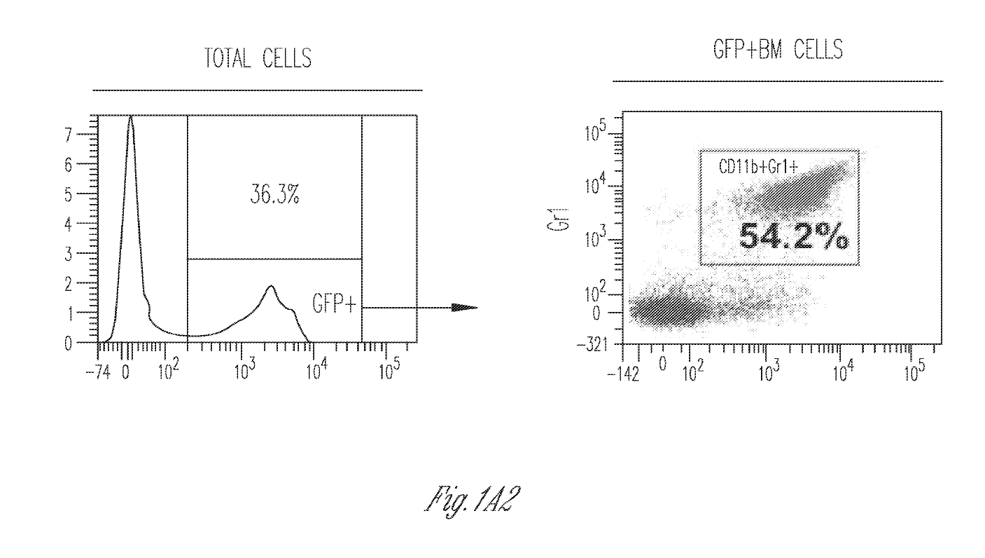

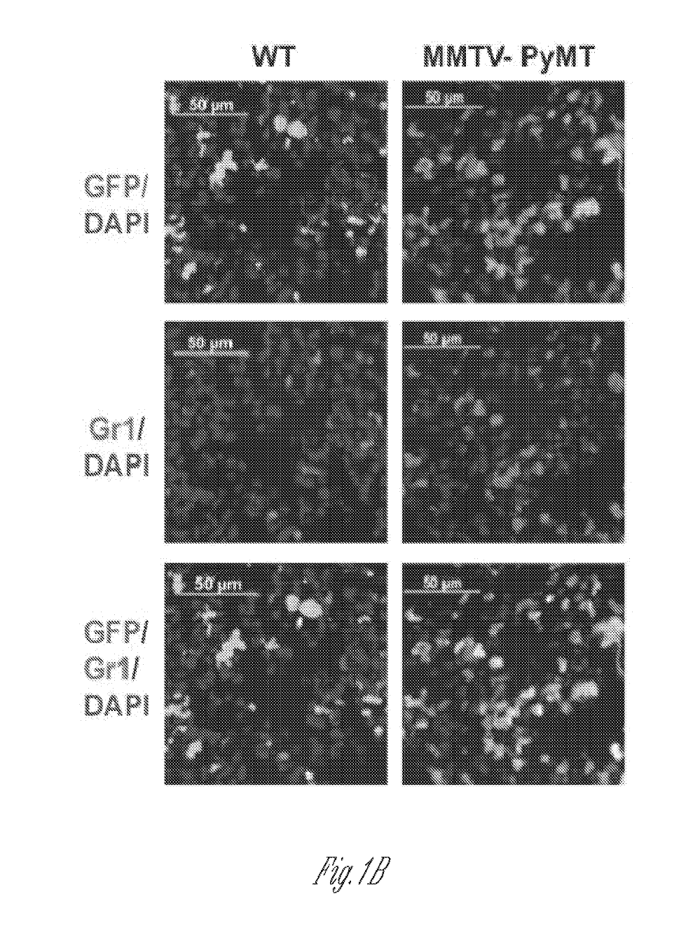

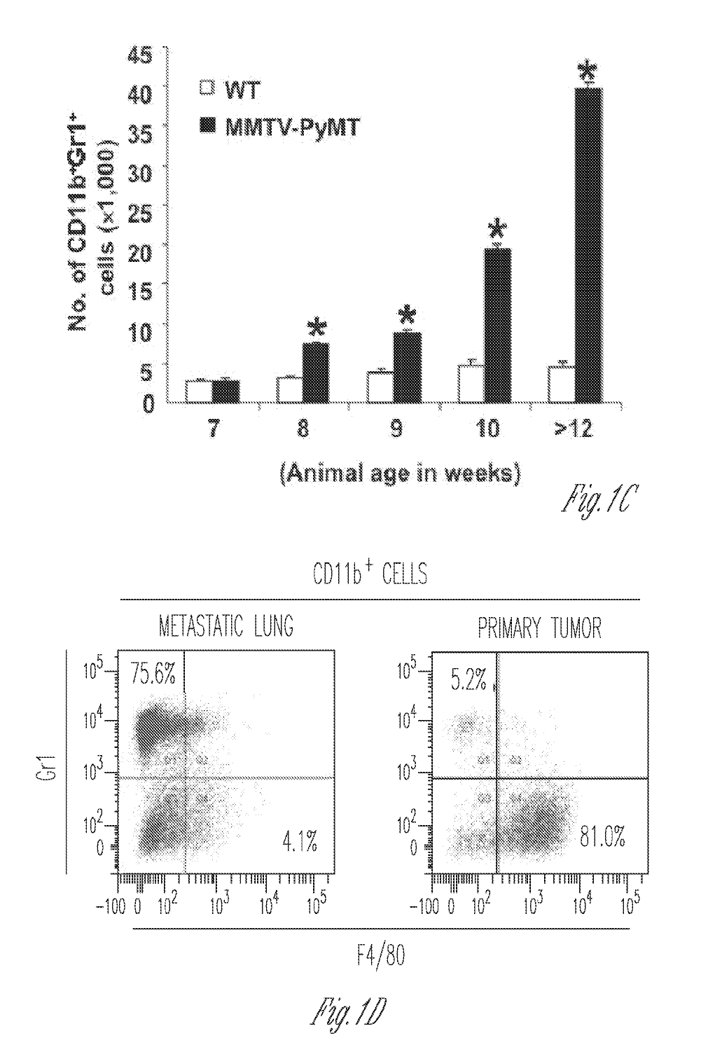

Flow cytometric analysis showed about 3-fold increased recruitment of GFP⁺ bone marrow-derived cells in the MMTV-PyMT metastatic lungs compared with wild-type (36.3%±4.3% and 12.4%±4.2% of total lung cells, respectively; FIG. 1A). Notably, in the metastatic lung, the recruited GFP⁺ bone marrow-derived cells were predominantly CD11b⁺Gr1⁺ myeloid progenitor cells (>50%; FIG. 1A) as determined by flow cytometry and immunostaining (FIG. 1B).

A kinetic analysis was performed to determine whether the recruitment of these cells was a function of metastases progression. Notably, the recruitment of CD11b⁺ Gr1⁺; cells was observed in the premetastatic lung of 8-week-old MMTV-PyMT mice before the appearance of metastases, and increasing numbers of CD11b⁺Gr1⁺ cells were associated with the progression of metastases (FIGS. 1C and 1E).

Conspicuously, compared with the metastatic lung, the numbers of CD11b⁺Gr1⁺ myeloid cells were less abundant in the primary tumor. The CD11b⁺ cells in primary tumor tissue were predominantly Gr1⁻F4/80⁺ macrophages (approximately 80%) in contrast to the Gr1⁺F4/80⁻ myeloid cells in metastatic lung (FIG. 1D). Such enhanced recruitment of myeloid cells specifically into metastatic lungs suggests that they may be involved in promoting outgrowth of tumor cells.

### Example 3

### Versican is Expressed by Myeloid Cells in the Metastatic Lung

To determine the molecular mechanisms by which the CD 11b⁺Gr1⁺ myeloid cells may contribute to lung metastasis, gene expression profiling was conducted of flow cytometry-sorted CD11b⁺Gr1⁺ cells from metastatic and wild-type lungs.

A cluster of differentially upregulated genes was identified in the myeloid cells from metastatic lungs. However, versican is an extracellular matrix chondroitin sulfate proteoglycan (Wight, Curr Opin Cell Biol 14:617-23 (2002), that is expressed by tumor stromal cells (Ricciardelli et al., Clin Cancer Res 8:1054-60 (2002); Suwiwat et al. Clin Cancer Res 10:2491-8 (2004); Pukkila et al., J Clin Pathol 60:267-72 (2007); Kodama et al., Ann Oncol 18:269-74 (2007); Pirinen et al., Hum Pathol 36:44-50 (2005); and Pukkila et al., J Clin Pathol 57:735-9 (2004)). While some information is available on versican, the biologic function of versican in vivo, particularly in the metastatic organs has not been elucidated. Versican was a focus of investigation in this Example.

RT-PCR analysis showed an approximately 5-fold increase in versican expression in the metastatic lung (ML, total) compared with controls (WT, total; FIG. 2A). In the metastatic lungs, versican expression was confined to CD11b⁺Gr1⁺ cells and not to the CD11b⁻Gr1⁻ stromal cells including subsets of T and B cells (FIG. 2B). Consistent with the RT-PCR results, Western blot analysis showed that versican protein expression was elevated in metastatic lung tissues compared with wild-type controls (FIG. 2B).

Flow cytometric analysis showed that the CD11b⁺Gr1⁺ cells are composed of CD11b⁺Ly6C^{high} and CD11b⁺Ly6G^{high} subpopulations (FIG. 2C) and their recruitment increased as a function of metastatic progression (FIGS. 2G and 2H). Interestingly, versican expression was confined to the Ly6C^{high} cells (FIG. 2D). Nuclear morphology analysis showed that the CD11b⁺Ly6C^{high} cells are mononuclear, whereas the CD11b⁺Ly6G^{high} cells are polymorphonuclear (FIG. 2E). Consistently, versican protein was also detected in the mononuclear CD11b⁺Ly6C^{high} cells by immunohistochemical (IHC) and Western blot analyses (FIGS. 2E and 2F). In the primary tumors, versican was also confined to the low abundance CD11b⁺Ly6C^{high} myeloid cells, whereas the abundant CD11b⁺F4/80⁺ myeloid cells did not express versican (FIG. 2I).

Contrary to previous studies using cultured cell lines (23-25), endothelial cells and the fibroblasts did not show significant versican levels compared with CD11b⁺Ly6C^{high} cells in the metastatic lungs (FIG. 2J). Importantly, the contribution of fibroblasts to the metastatic lung was about 10-fold lower compared with CD11b⁺Ly6C^{high} cells, and no significant increase in numbers was observed in the metastatic lungs compared with controls (FIG. 2K). Furthermore, analysis a panel of tumor cell lines showed significantly lower versican expression compared with sorted myeloid cells (FIG. 2L). Taken together, these results indicate that the Ly6C^{high} myeloid cells are the major contributors of versican in metastatic lungs.

### Example 4

### Versican Suppression in Myeloid Cells Impairs Lung Metastases

To explore the role of myeloid cell-derived versican in pulmonary metastasis, versican expression was knocked down in bone marrow cells in vivo.

Given that versican expression is essentially confined to the CD11b⁺Ly6C^{high} myeloid cells, versican knockdown in total bone marrow cells would largely only impact Ly6C^{high} cells. Thus, the impact of versican expression on CD11b⁺Ly6C^{high} could be examined with specificity. Two shRNAs were generated that specifically target exon 8, which is the versican V0/V1-specific exon (FIGS. 3A and 3B). These shRNAs effectively reduced endogenous versican (V0/V1) expression as compared with nonspecific shRNA control (FIG. 3C).

Versican-specific shRNA (shVcn) or nonspecific shRNA (shNS) was introduced via lentiviruses into lineage wild type-negative (Lin⁻) bone marrow progenitor cells and the cells were then transplanted into lethally irradiated MMTV-PyMT recipient mice (4 weeks old) as described in our previous studies (Gao et al., Science 319:195-198 (2008); Nolan et al., Genes Dev 21:1546-58 (2007)). Thus, shVcn-bone marrow transplant mice were generated that expressed the versican shRNA and shNS-bone marrow transplant MMTV-PyMT mice were generated that expressed the non-specific shRNA. Successful bone marrow reconstitution was confirmed by flow cytometric analysis of transplanted recipient mice (FIG. 3D).

Versican expression was upregulated (>3-fold) in the lungs of shNS-bone marrow transplant MMTV-PyMT mice compared with controls (FIG. 3E). However, versican expression was inhibited in shVcn-bone marrow transplant mice (FIG. 3E). Versican knockdown in the recruited Gr1+ cells in the lungs of shVcn-bone marrow transplant mice (FIGS. 3F and 3G), resulted in reduced lung metastases compared with shNS-bone marrow transplant controls (0.60%±0.25% vs. 12.8%±3.2%, respectively; FIGS. 3H and 3I). Immunohistochemical examination of the lungs from shVcn-bone marrow transplant animals indicated that there was severe impairment of macrometastases while micrometastases remained unaffected (FIGS. 3J and 3M). The primary breast tumors remained unaffected in these mice.

Taken together, these results show that versican deficiency in the recruited myeloid cells significantly impaired tumor outgrowth at the metastatic site.

### Example 5

### Versican Knockdown does not Perturb the Immune Microenvironment of the Lungs

Versican knockdown did not affect the recruitment of CD11b⁺Gr1⁺ myeloid cells in the lung microenvironment as determined by immunostaining (FIG. 3F) and flow cytometry (15.8%±1.9% in shRNA-bone marrow transplant vs. 15.3%±4.2% in shVcn-bone marrow transplant mice; FIGS. 3N and 3O). Versican knockdown also did not perturb the recruitment of other bone marrow cells including B cells (B220⁺) or T cells (CD3⁺; FIGS. 3N and 3O). The immune microenvironment of the lungs remained unperturbed as a result of versican knockdown as evaluated by expression of key mediators including TNF-α, interleukin-1 (IL-1), interleukin-6 (IL-6), interleukin-4 (IL-4), interleukin-10 (IL-10), arginase 1, arginase 2, and NOS2 (FIG. 3P). These results indicate that versican deficiency did not affect the myeloid-derived suppressor cell (MDSC) activity of myeloid cells.

### Example 6

### Myeloid Cell-Derived Versican Enhances Proliferation of Metastatic Tumor Cells to Promote Outgrowth

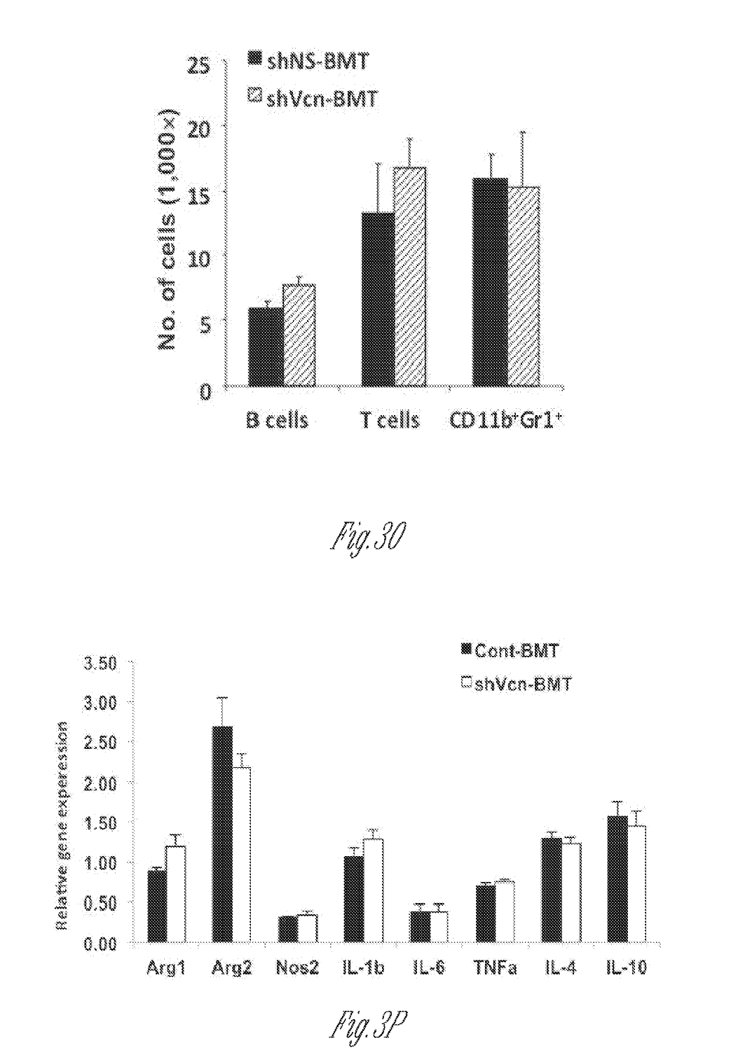

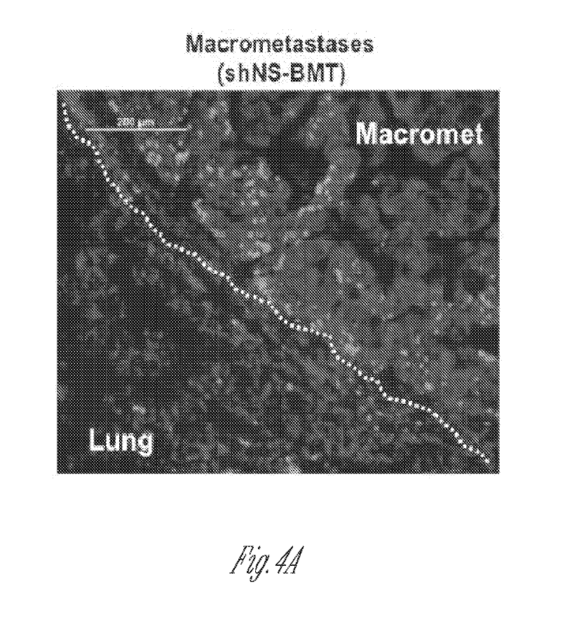

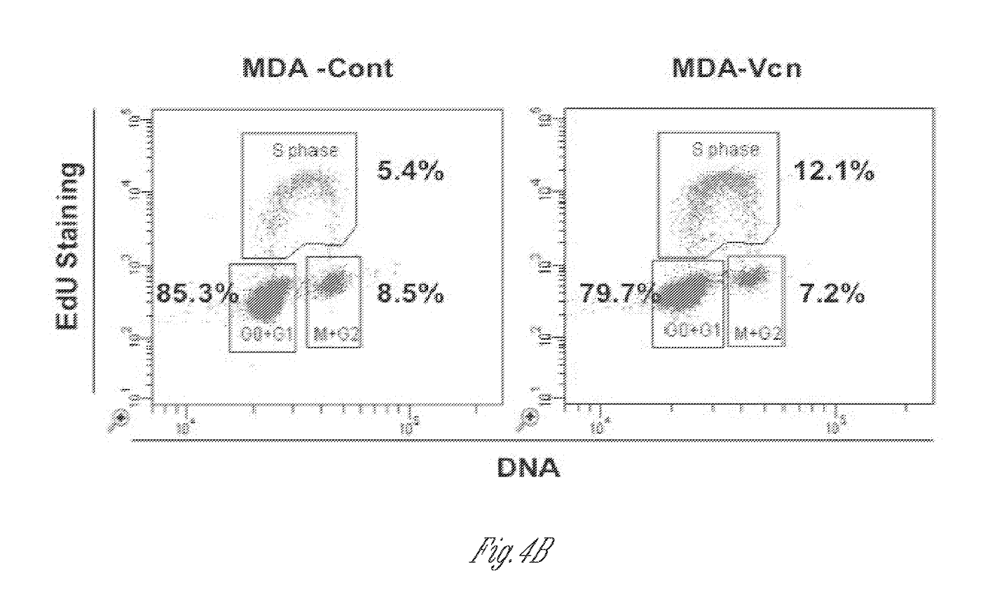

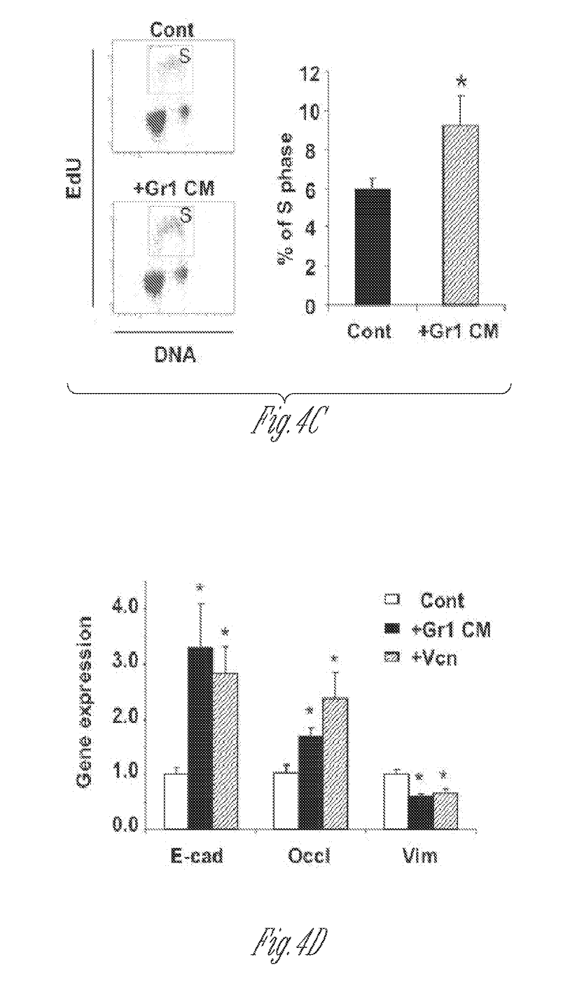

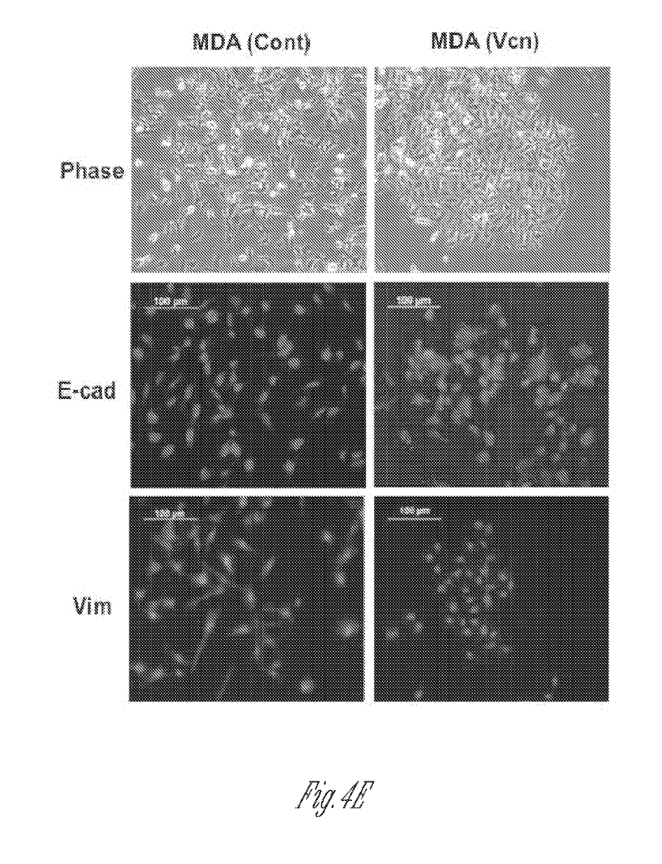

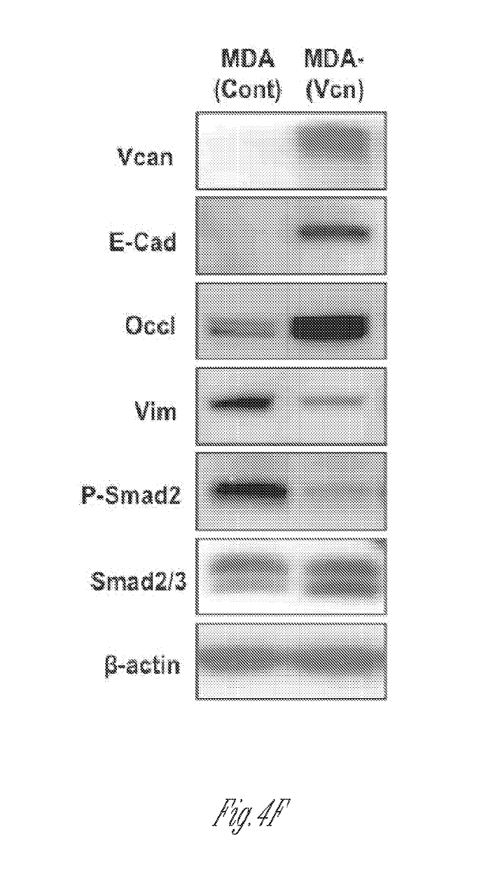

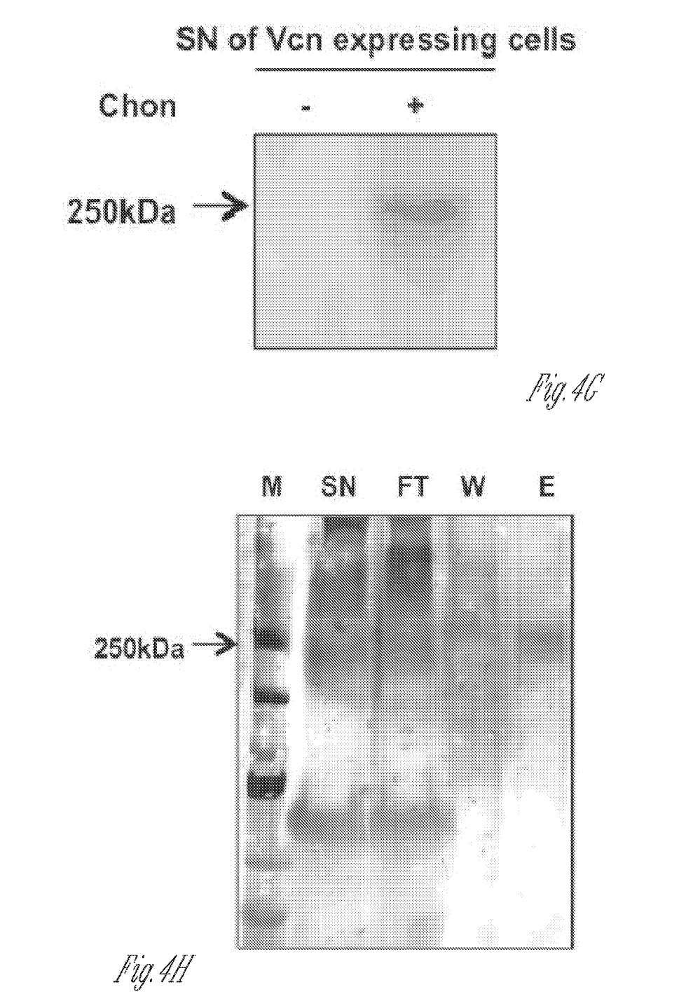

The results described herein indicate that myeloid progenitor cells have a novel function in promoting metastases. The inventors hypothesize that versican expressed by recruited myeloid cells can enhance proliferation of metastatic tumor cells to promote tumor outgrowth. Consistent with this hypothesis, abundant Ki67⁺ proliferating cells were observed in metastatic lesions in shNS-bone marrow transplant mice where versican was available, compared with metastatic lesions from shVcn-bone marrow transplant mice where versican expression was suppressed (FIGS. 3F and 3G; FIG. 4A).

Taken together, these results show that versican deficiency in the bone marrow does not affect the recruitment of CD11b⁺Gr1⁺ cells and other immune cells to the lung microenvironment. However, versican expressed by these myeloid cells promotes metastatic tumor outgrowth by enhancing cell proliferation as discussed in further detail below.

### Example 7

### Versican Promotes Proliferation and Induces Mesenchymal to Epithelial Transition of Metastatic Tumor Cells

This Example illustrates that versican not only promotes metastatic tumor cell growth but also induces mesenchymal to epithelial transition of metastatic tumor cells.

To further evaluate the cell proliferation-promoting function of versican, metastatic human breast cancer MDA-MB-231 cells were employed. Conditioned media was generated from flow cytometry-sorted CD11b⁺Gr1⁺ cells from the lungs of tumor-bearing mice and this conditioned media was used to mirror the paracrine effects of versican secreted by myeloid cells on metastatic tumor cells in vitro. Administration of the CD11b⁺Gr1⁺ conditioned media to MDA-MB-231 cells increased the percentage of cells in S-phase compared with controls (FIG. 4C). Consistently, the proliferation rate of MDA-MB-231 cells was enhanced following expression of a secreted form of versican V1 isoform (12.1% and 5.4% of S-phase cells with and without versican, respectively; FIG. 4B).

Interestingly, the versican-induced proliferation increase in MDA-MB-231 cells treated with CD11b⁺Gr1⁺ conditioned media was associated with the acquisition of an epithelial phenotype as indicated by upregulation of epithelial cell markers including E-cadherin and occludin and a concomitant inhibition of the mesenchymal marker vimentin (FIG. 4D). These results suggested indicated that mesenchymal-to-epithelial transition had occurred in these cells, and that a factor present in the CD11b⁺Gr1⁺ conditioned media mediated such transition.

To ascertain whether versican was the factor responsible for mesenchymal-to-epithelial transition of MDA-MB-231 cells, the CD11b⁺Gr1⁺ conditioned media was analysed to determine whether versican was present and to purify any such versican from the conditioned media. As shown in FIG. 4G, versican V1 isoform was detected in the CD11b⁺Gr1⁺ conditioned media after chondroitinase ABC (Chon) treatment and removal of debris. The versican (V1 isoform) was purified from the conditioned media (FIGS. 4G and 4H), and the MDA-MB-231 cells were treated with biochemically purified versican. Induction of mesenchymal to epithelial transition was monitored. As shown in FIG. 4E, MDA-MB-231 cells that were exposed to the secreted form of versican (V1 isoform) established aggregated cobblestone-like colonies (FIG. 4E, phase-contrast; top and bottom) and showed induction of epithelial and suppression of mesenchymal markers as determined by immunostaining, Western blot, and RT-PCR analysis (FIGS. 4E, 4F and 4I). Importantly, versican attenuated phospho (p)-Smad2 levels in MDA-MB-231 cells, whereas the levels of total Smad2/3 remained unchanged (FIG. 4F). Phosphorylated-Smad2 is a regulator of key epithelial-to-mesenchymal-transition (EMT) promoting transcription factors including Snail. Versican-mediated attenuation of p-Smad2 levels (FIG. 4F) and suppression of Snail (FIG. 4I) in MDA-MB-231 cells may therefore have inhibited the TGF-β/Smad2/3 signalling pathway, a well-known stimulator of epithelial to mesenchymal transition (EMT) in various tumors (Padua et al., Cell Res 19:89-102 (2009); Shi et al., Cell 113:685-700 (2003); Song, Cell Res 17:289-90 (2007); Hata et al., Mol Med Today 4:257-62 (1998)). These results indicate that versican-mediated blockade of the TGF-β/Smad2 pathway may stimulate mesenchymal-to-epithelial transition, resulting in increased cell proliferation that collectively promotes focal tumor outgrowth at the metastatic site.

### Example 8

### Versican Deficiency Inhibits Metastases In Vivo by Blocking Mesenchymal-to-Epithelial Transition

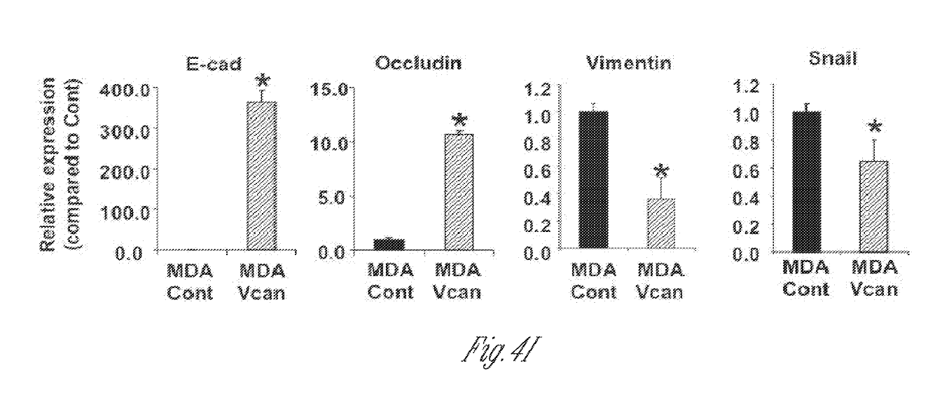

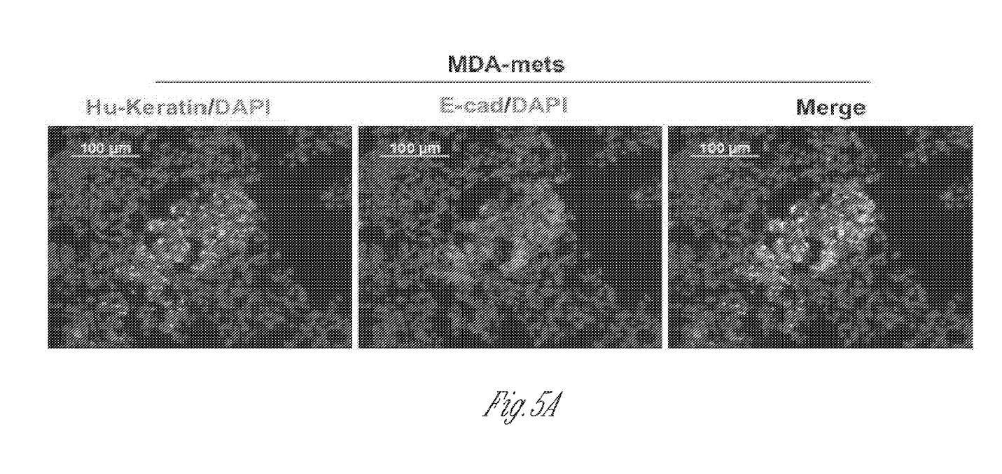

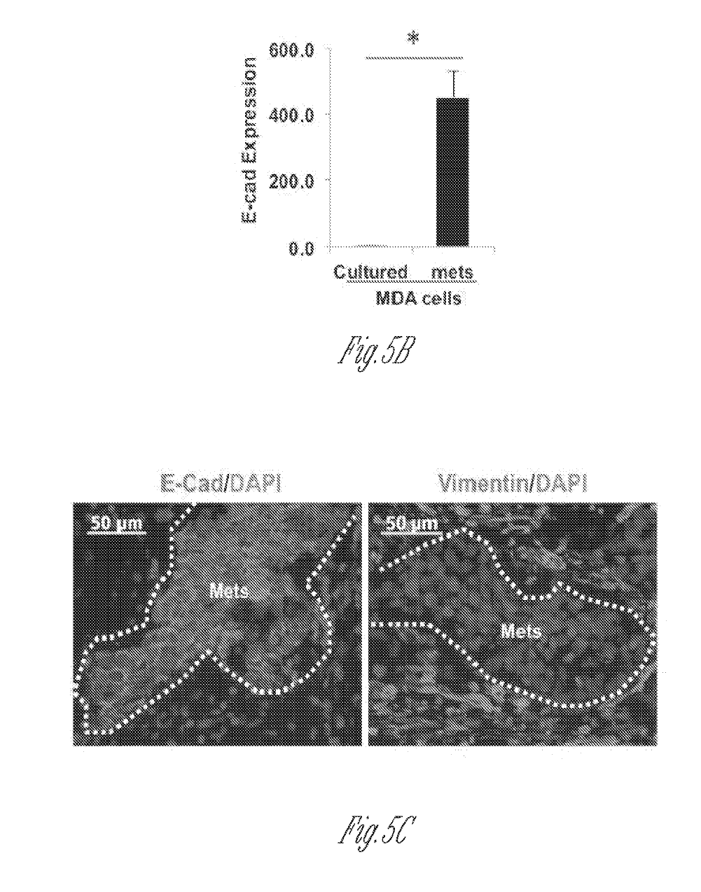

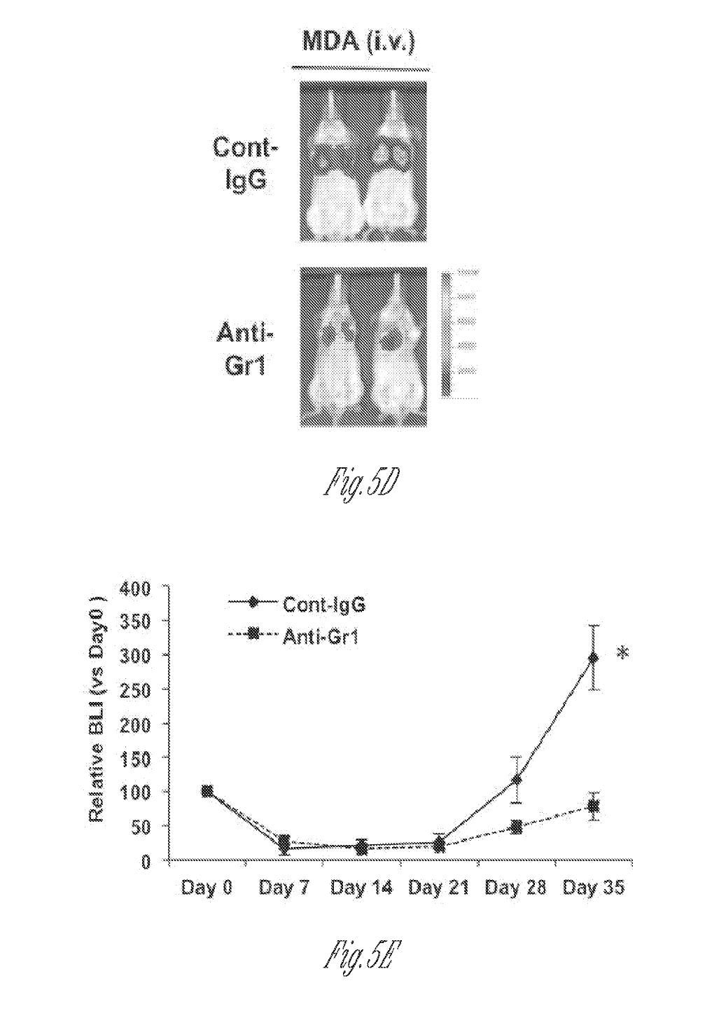

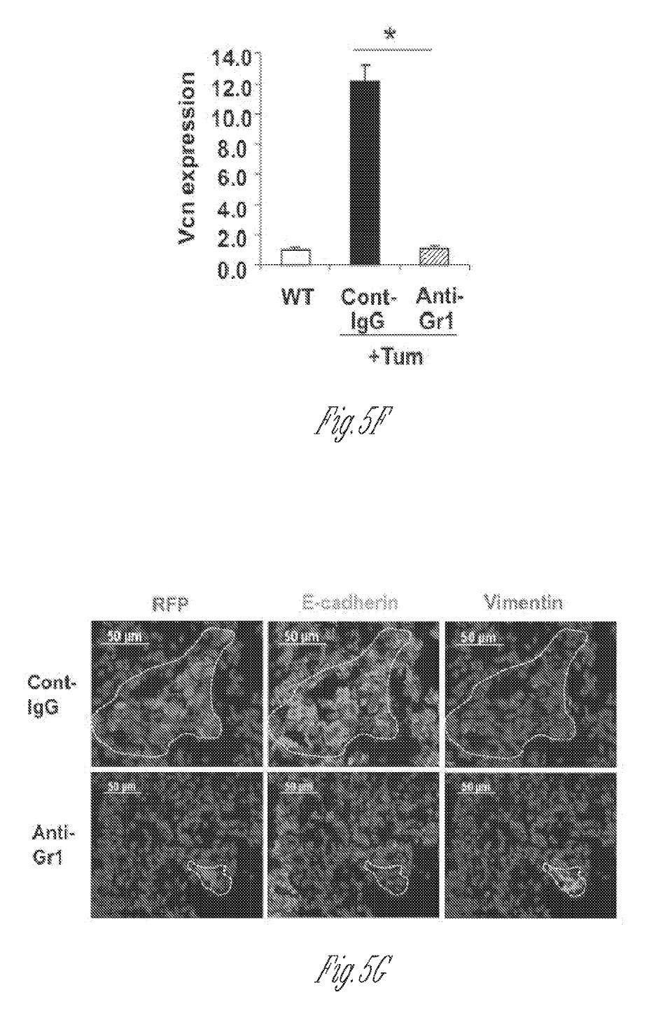

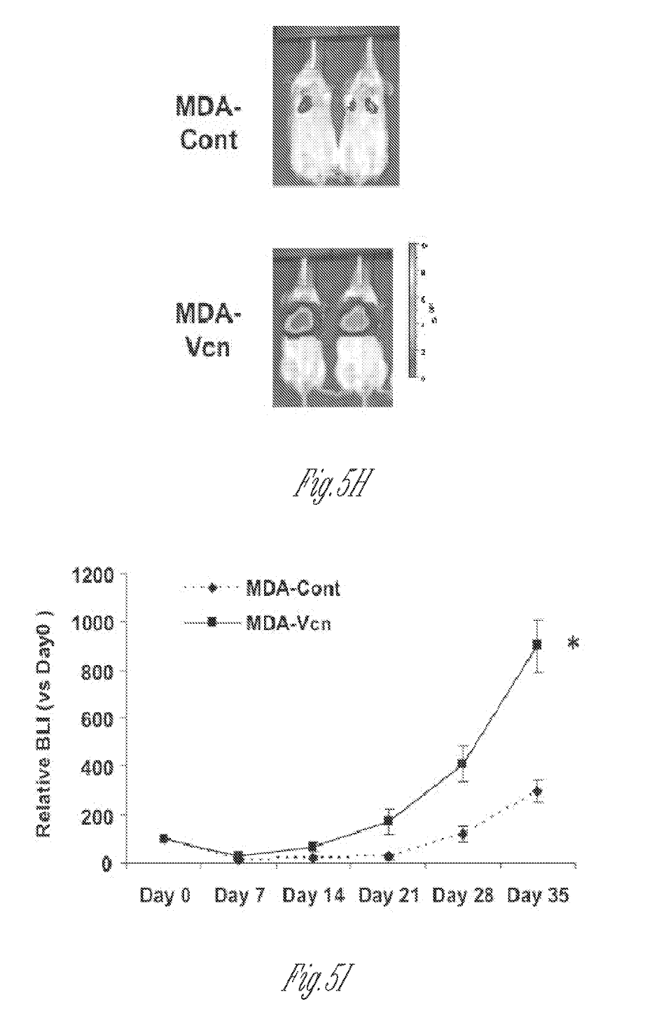

In tumor progression, epithelial-to-mesenchymal-transition (EMT) confers an invasive and metastatic phenotype that supports escape of tumor cells from the primary tumor site. It is speculated that subsequently, the disseminated mesenchymal tumor cells must undergo the reverse transition, mesenchymal-to-epithelial transition (MET), at the site of metastasis, as metastases recapitulate the pathology of their corresponding primary tumors. However, mesenchymal-to-epithelial transition has not been accurately recapitulated in breast cancer metastasis. To assess if mesenchymal-to-epithelial transition occurs in vivo, severe combined immunodeficient (SCID) mice were inoculated with MDA-MB-231 cells that exhibit a typical mesenchymal phenotype (E-cadherin⁻/vimentin⁺) in and allowed metastases to develop in the lungs. Notably, MDA-derived metastases exhibited an E-cadherin+ epithelial phenotype (FIGS. 5A and 5B), suggesting that MDAMB-231 cells have undergone MET in the lung environment. In this context, analysis of lung metastases from patients with breast cancer also showed E-cadherin⁺ and vimentin⁻ metastases (FIG. 5C). On the basis of these observations, the inventors hypothesized that myeloid cells recruited in the premetastatic lungs produce versican which induces mesenchymal-to-epithelial transition (MET) in tumor cells to promote tumor outgrowth.

To determine whether versican-mediated MET is necessary for metastases formation in vivo, luciferase-RFP-labelled MDA-MB-231 cells were injected into the tail vein of SCID mice and the mice were monitored for metastases progression either in the presence of versican (control IgG-treated mice) or in lack of versican-producing myeloid cells (anti-Gr1-treated mice). FIGS. 5D and 5E show that depletion of Gr1⁺ myeloid cells significantly reduced progression of metastases by more than 5-fold. Of note, the anti-Gr1 antibody treatment significantly inhibited the upregulated versican expression in the metastatic lungs (FIG. 5F).

Further evaluation of the lungs showed that macroscopic E-cadherin⁺/vimentin^{low} metastatic lesions were generated by MDA-MB-231 cells in the presence of versican (FIG. 5G, top) as expected. However, versican deficiency resulted in microscopic vimentin^{high}/E-cadherin⁻ lesions (FIG. 5G, bottom) indicating a failure of mesenchymal-to-epithelial transition (MET). To confirm that the MET-induced accelerated metastasis was mainly due to versican, a versican gain-of-function experiment was performed. SCID mice were injected via the tail vein with MDAMB-231 cells expressing a secreted form of versican V1 isoform (MDA-Vcn) or MDA-Cont cells that did not express versican. Bioluminescence imaging analysis revealed accelerated progression (>4-fold) of lung metastases with MDA-Vcn cells compared with MDA-Cont cells (FIGS. 5H and 5I).

Taken together, these results indicate that versican promotes mesenchymal-to-epithelial transition of metastatic tumor cells and enhances progression into macrometastatic lesions.

### Example 9

### Myeloid Cells Express Versican in the Metastatic Lungs of Breast Cancer Patients

This Example describes analysis of metastatic lungs from patients with breast cancer to ascertain whether lung tissues exhibit elevated levels of versican as was observed in experiments with the mouse models.

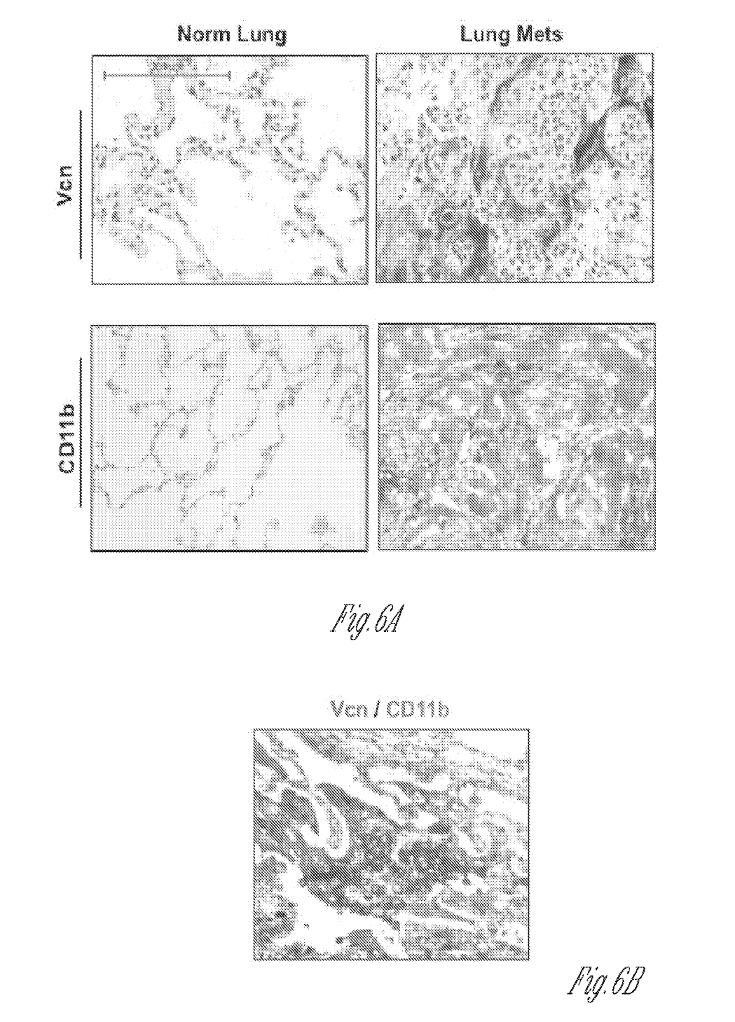

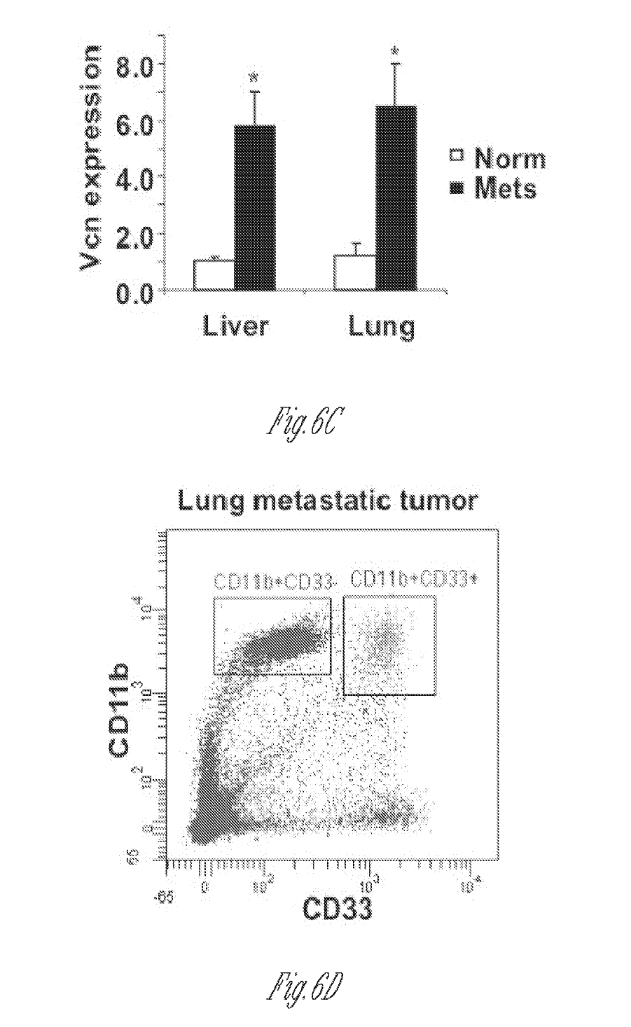

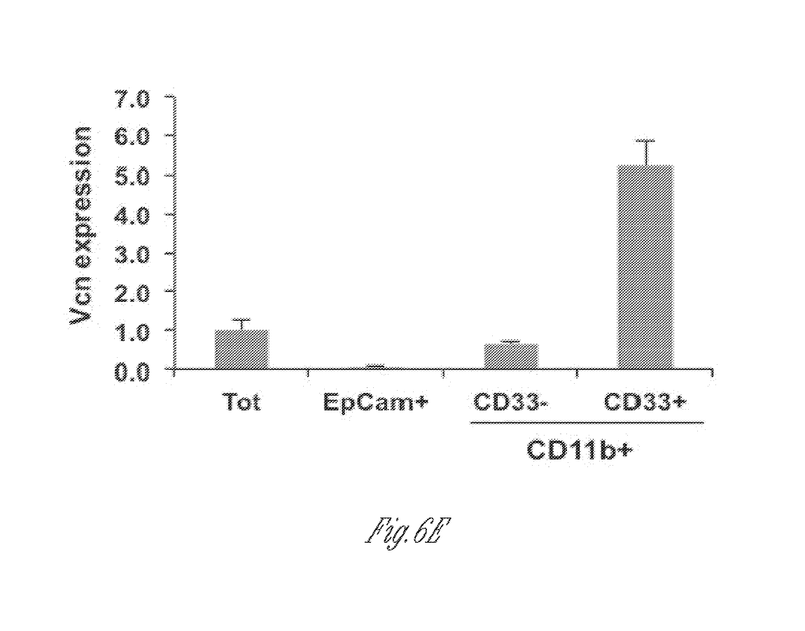

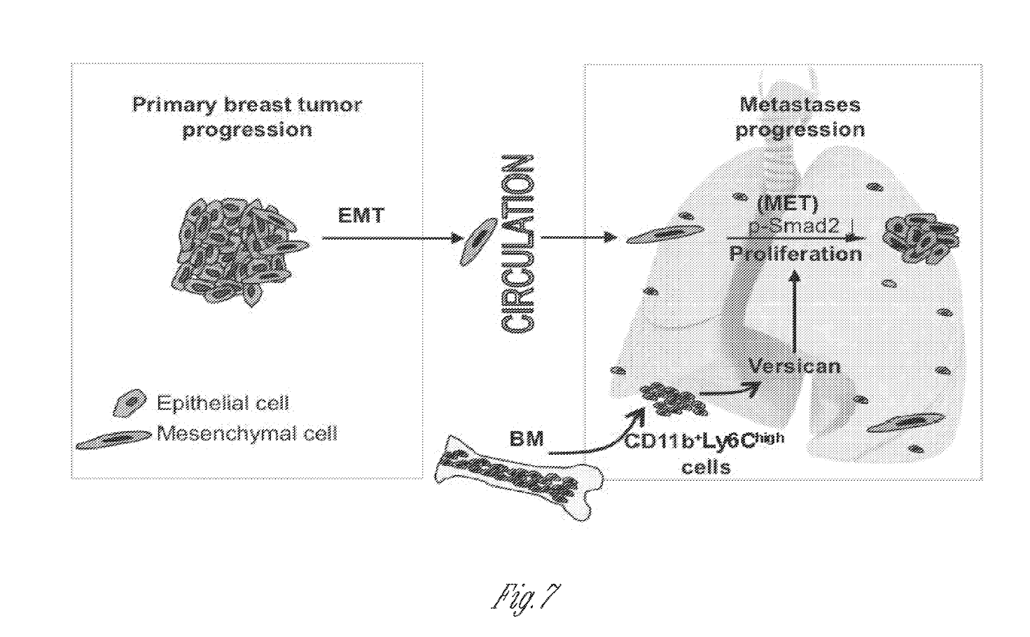

Immunohistochemical analysis showed that the metastatic lungs of patients with breast cancer exhibited enhanced versican expression (FIG. 6A); however, the lungs of normal healthy controls did not exhibit enhanced versican expression (FIG. 6A).

Immunohistochemical analysis of metastatic lungs also showed that versican expression was confined to the vicinity of CD11b⁺ myeloid cell clusters (FIG. 6B). Versican expression was quantified by RT-PCR in a cohort of patients with breast cancer who had developed lung (n=6) or liver (n=11) metastases. Significantly higher versican expression was detected in metastatic organs than in normal tissues (5.8±1.3 and 6.5±1.5-fold in the liver and lung metastasis, respectively; FIG. 6C). A subset of CD11b+ cells that expressed versican in patients with cancer was further evaluated. Humans myeloid cells were defined by the coexpression of CD11b and CD33 (Krystal et al., Cancer Res 67:3986 (2007). Flow cytometric analysis showed that the CD11b⁺CD33⁻ cells that comprise the monocytic population expressed versican, whereas the CD11b⁺CD33⁻ fraction or the pure tumor cells did not (FIGS. 6D and E). These results are consistent with the observations described above in mouse models, and show that versican is expressed by tumor-elicited myeloid cells may contribute to metastasis in patients with cancer.

### REFERENCES

1. Townson J L, Chambers A F. Dormancy of solitary metastatic cells. Cell Cycle 2006; 5:1744-50.
2. Naumov G N, Bender E, Zurakowski D, Kang S Y, Sampson D, Flynn E, et al. A model of human tumor dormancy: an angiogenic switch from the nonangiogenic phenotype. J Natl Cancer Inst 2006; 98:316-25.
3. Gupta G P, Massague J. Cancer metastasis: building a framework. Cell 2006; 127:679-95.
4. Joyce J A, Pollard J W. Microenvironmental regulation of metastasis. Nat Rev Cancer 2009; 9:239-52.
5. Polyak K, Weinberg R A. Transitions between epithelial and mesenchymal states: acquisition of malignant and stem cell traits. Nat Rev Cancer 2009; 9:265-73.
6. Fidler I J. The pathogenesis of cancer metastasis: the ‘seed and soil’ hypothesis revisited. Nat Rev Cancer 2003; 3:453-8.
7. Kaplan R N, Psaila B, Lyden D. Bone marrow cells in the ‘pre-metastatic niche’: within bone and beyond. Cancer Metastasis Rev 2006; 25: 521-9.
8. Gao D, Nolan D J, Mellick A S, Bambino K, McDonnell K, Mittal V. Endothelial progenitor cells control the angiogenic switch in mouse lung metastasis. Science 2008; 319:195-8.
9. Chaffer C L, Thompson E W, Williams E D. Mesenchymal to epithelial transition in development and disease. Cells Tissues Organs 2007; 185:7-19.
10. Dutt S, Cassoly E, Dours-Zimmermann M T, Matasci M, Stoeckli E T, Zimmermann D R. Versican V0 and V1 direct the growth of peripheral axons in the developing chick hindlimb. J Neurosci 2011; 31:5262-70.
11. Guy C T, Cardiff R D, Muller W J. Induction of mammary tumors by expression of polyomavirus middle T oncogene: a transgenic mouse model for metastatic disease. Mol Cell Biol 1992; 12:954-61.
12. Nolan D J, Ciarrocchi A, Mellick A S, Jaggi J S, Bambino K, Gupta S, et al. Bone marrow-derived endothelial progenitor cells are a major determinant of nascent tumor neovascularization. Genes Dev 2007; 21:1546-58.
13. Wight T N. Versican: a versatile extracellular matrix proteoglycan in cell biology. Curr Opin Cell Biol 2002; 14:617-23.
14. Ricciardelli C, Brooks J H, Suwiwat S, Sakko A J, Mayne K. Raymond W A, et al. Regulation of stromal versican expression by breast cancer cells and importance to relapse-free survival in patients with nodenegative primary breast cancer. Clin Cancer Res 2002; 8:1054-60.
15. Suwiwat S, Ricciardelli C, Tammi R, Tammi M, Auvinen P, Kosma V M, et al. Expression of extracellular matrix components versican, chondroitin sulfate, tenascin, and hyaluronan, and their association with disease outcome in node-negative breast cancer. Clin Cancer Res 2004; 10:2491-8.
16. Pukkila M, Kosunen A, Ropponen K, Virtaniemi J, Kellokoski J, Kumpulainen E, et al. High stromal versican expression predicts unfavourable outcome in oral squamous cell carcinoma. J Clin Pathol 2007; 60:267-72.
17. Kodama J, Hasengaowa, Kusumoto T, Seki N, Matsuo T, Ojima Y, et al. Prognostic significance of stromal versican expression in human endometrial cancer. Ann Oncol 2007; 18:269-74.
18. Pirinen R, Leinonen T, Bohm J, Johansson R, Ropponen K, Kumpulainen E, et al. Versican in nonsmall cell lung cancer: relation to hyaluronan, clinicopathologic factors, and prognosis. Hum Pathol 2005; 36:44-50.
19. Pukkila M J, Kosunen A S, Virtaniemi J A, Kumpulainen E J, Johansson R T, Kellokoski J K, et al. Versican expression in pharyngeal squamous cell carcinoma: an immunohistochemical study. J Clin Pathol 2004; 57:735-9.
20. Krystal G, Sly L, Antignano F, Ho V, Ruschmann J, Hamilton M. Re: The terminology issue for myeloid-derived suppressor cells. Cancer Res 2007; 67:3986.
21. Gabrilovich D I, Bronte V, Chen S H, Colombo M P, Ochoa A, Ostrand-Rosenberg S, et al. The terminology issue for myeloid-derived suppressor cells. Cancer Res 2007; 67:425; author reply 426.
22. Coffelt S B, Lewis C E, Naldini L, Brown J M, Ferrara N, De Palma M. Elusive identities and overlapping phenotypes of proangiogenic myeloid cells in tumors. Am J Pathol 2010; 176:1564-76.
23. Ricciardelli C, Sakko A J, Ween M P, Russell D L, Horsfall D J. The biological role and regulation of versican levels in cancer. Cancer Metastasis Rev 2009; 28:233-45.
24. Zimmermann D R, Ruoslahti E. Multiple domains of the large fibroblast proteoglycan, versican. EMBO J. 1989; 8:2975-81.
25. Sheng W, Wang G, Wang Y, Liang J, Wen J, Zheng P S, et al. The roles of versican V1 and V2 isoforms in cell proliferation and apoptosis. Mol Biol Cell 2005; 16:1330-40.
26. Padua D, Massague J. Roles of TGFbeta in metastasis. Cell Res 2009; 19:89-102.
27. Shi Y, Massague J. Mechanisms of TGF-beta signaling from cell membrane to the nucleus. Cell 2003; 113:685-700.
28. Song J. EMT or apoptosis: a decision for TGF-beta. Cell Res 2007; 17:289-90.
29. Hata A, Shi Y, Massague J. TGF-beta signaling and cancer: structural and functional consequences of mutations in Smads. Mol Med Today 1998; 4: 257-62.
30. Hugo H. Ackland M L, Blick T, Lawrence M G, Clements J A. Williams E D, et al. Epithelial—mesenchymal and mesenchymal—epithelial transitions in carcinoma progression. J Cell Physiol 2007; 213:374-83.
31. Chaffer C L, Brennan J P, Slavin J L, Blick T, Thompson E W, Williams E D. Mesenchymal-to-epithelial transition facilitates bladder cancer metastasis: role of fibroblast growth factor receptor-2. Cancer Res 2006; 66:11271-8.
32. Kowanetz M, Wu X, Lee J, Tan M, Hagenbeek T, Qu X, et al. Granulocyte-colony stimulating factor promotes lung metastasis through mobilization of Ly6G [symbol] Ly6C [symbol] granulocytes. Proc Natl Acad Sci USA 2010; 107:21248-55.
33. Erler J T, Bennewith K L, Cox T R, Lang G, Bird D, Koong A, et al. Hypoxia-induced lysyl oxidase is a critical mediator of bone marrow cell recruitment to form the premetastatic niche. Cancer Cell 2009; 15:35-44.
34. Yang L, Huang J, Ren X, Gorska A E, Chytil A, Aakre M, et al. Abrogation of TGF beta signaling in mammary carcinomas recruits Gr-1⁺CD11b⁺ myeloid cells that promote metastasis. Cancer Cell 2008; 13:23-35.
35. Ostrand-Rosenberg S, Sinha P. Myeloid-derived suppressor cells: linking inflammation and cancer. J Immunol 2009; 182:4499-506.
36. Youn J I, Nagaraj S, Collazo M, Gabrilovich D I. Subsets of myeloid-derived suppressor cells in tumor-bearing mice. J Immunol 2008; 181: 5791-802.
37. Kim S, Takahashi H, Lin W W, Descargues P, Grivennikov S, Kim Y, et al. Carcinoma-produced factors activate myeloid cells through TLR2 to stimulate metastasis. Nature 2009; 457:102-6.
38. Vainio S, Lin Y. Coordinating early kidney development: lessons from gene targeting. Nat Rev Genet. 2002; 3:533-43.
39. Prudkin L, Liu D D, Ozburn N C, Sun M, Behrens C, Tang X, et al. Epithelial-to-mesenchymal transition in the development and progression of adenocarcinoma and squamous cell carcinoma of the lung. Mod Pathol 2009; 22:668-78.
40. Mur C. Martinez-Carpio P A, Fernandez-Montoli M E, Ramon J M, Rosel P, Navarro M A. Growth of MDA-MB-231 cell line: different effects of TGF-beta(1), EGF and estradiol depending on the length of exposure. Cell Biol Int 1998; 22:679-84.
41. Zugmaier G, Ennis B W, Deschauer B, Katz D, Knabbe C, Wilding G, et al. Transforming growth factors type beta 1 and beta 2 are equipotent growth inhibitors of human breast cancer cell lines. J Cell Physiol 1989; 141:353-61.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “an antibody” or “a nucleic acid” or “a polypeptide” includes a plurality of such antibodies, nucleic acids or polypeptides (for example, a solution of antibodies, nucleic acids or polypeptides or a series of antibody, nucleic acid or polypeptide preparations), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. §1.72(b) to allow the reader to quickly ascertain the nature and gist of the technical disclosure. The Abstract is submitted with the understanding that it will not be used to interpret or limit the scope or meaning of the claims.

The following statements of the invention are intended to describe some elements of the invention.

### Statements Describing Aspects of the Invention:

1. A method of inhibiting establishment or growth of metastatic tumor cells at a site distal from a primary tumor in an animal comprising administering to the animal a composition comprising a versican inhibitor to thereby inhibit establishment or growth of metastatic tumor cells at a site distal from a primary tumor in the animal.
2. The method of statement 1, wherein the versican inhibitor does not affect growth of the primary tumor.
3. The method of statement 1 or 2, wherein the animal has undergone surgery to remove the primary tumor.
4. The method of any of statements 1-3, wherein the animal has undergone surgery to remove the primary tumor and at least one metastatic tumor.
5. The method of any of statements 1-4, wherein the versican inhibitor is administered to bone marrow.
6. The method of any of statements 1-5, wherein the versican inhibitor is formulated to target bone marrow or bone marrow-derived cells.
7. The method of any of statements 1-6, wherein the versican inhibitor is formulated to target myeloid progenitor cells.
8. The method of any of statements 1-7, wherein the versican inhibitor is formulated to target monocytes.
9. The method of any of statements 1-8, wherein the versican inhibitor is formulated to target macrophages.
10. The method of any of statements 1-9, wherein the versican inhibitor is formulated in a liposomal or nanoparticle carrier.
11. The method of any of statements 1-10, wherein the versican inhibitor inhibits versican expression in bone marrow cells of the animal.
12. The method of any of statements 1-11, wherein the method inhibits recruitment of myeloid progenitor cells to a premetastatic site in the animal.
13. The method of any of statements 1-12, wherein the method inhibits TGF-β/Smad2/3 signaling in the animal.
14. The method of any of statements 1-13, wherein the composition further comprises an antibody that specifically binds to CD11b, CD33, VEGF receptor, AFP, CEA, CA-125, MUC-1, ETA, tyrosinase, ras, p53, MAGE 1, or combinations of antibodies that specifically bind to any of CD11b, CD33, VEGF receptor, AFP, CEA, CA-125, MUC-1, ETA, tyrosinase, ras, p53, and MAGE1.
15. The method of statements 14, wherein the anti-CD11b antibody or the anti-CD33 antibody binds to myeloid cells.
16. The method of any of statements 1-15, wherein the composition comprises a versican inhibitor selected from the group consisting of budesonide, one or more hyaluronan oligomers, one or more anti-versican antibodies, one or more non-functioning versican peptides, one or more versican inhibitory nucleic acids, and combinations thereof.
17. The method of any of statements 1-16, wherein the composition comprises a versican inhibitory nucleic acid that can specifically bind to a versican mRNA under physiological conditions.
18. The method of any of statements 1-17, wherein the composition comprises a versican inhibitory nucleic acid that can specifically bind to a versican mRNA comprising SEQ ID NO:3 under physiological conditions.
19. The method of any of statements 1-18, wherein the composition comprises at least one versican inhibitory nucleic acid that can inhibit expression or translation of a versican mRNA.
20. The method of any of statements 1-19, wherein the composition comprises at least one versican inhibitory nucleic acid with a sequence comprising 5′-ACACCAGAATTAG AAAGTTCAA-3′ (shVcn1; SEQ ID NO:43), or 5′-AGCACCTTGTCTGATGGCCAAG-3′ (shVcn2; SEQ ID NO:44).
21. The method of any of statements 1-19, wherein the composition comprises at least one versican inhibitory nucleic acid with an RNA sequence corresponding to a DNA sequence comprising 5′-ACACCAGAATTAG AAAGTTCAA-3′ (shVcn 1; SEQ ID NO:43), or 5′-AGCACCTTGTCTGATGGCCAAG-3′ (shVcn2; SEQ ID NO:44).
22. The method of any of statements 1-19, wherein the composition comprises at least one versican inhibitory nucleic acid with a DNA or RNA sequence comprising a sequence complementary to 5′-ACACCAGAATTAG AAAGTTCAA-3′ (shVcn1; SEQ ID NO:43), or 5′-AGCACCTTGTCTGATGGCCAAG-3′ (shVcn2; SEQ ID NO:44).
23. The method of any of statements 1-22, wherein the composition comprises at least one versican inhibitory peptide.
24. The method of any of statements 1-23, wherein the composition comprises at least one versican inhibitory peptide with a sequence that has at least 90% sequence identity to a sequence selected from the group consisting of SEQ ID NO:4-41 and 42.
25. The method of any of statements 1-24, wherein the composition comprises at least one versican inhibitory peptide with a sequence comprising an amino acid sequence selected from the group consisting of SEQ ID NO:4-41 and 42.
26. The method of any of statements 1-25, wherein the composition further comprises an additional therapeutic agent or anti-cancer agent.
27. The method of statement 26, wherein the additional therapeutic agent or anti-cancer agent is selected from the group consisting of a radioactive drug, topoisomerase inhibitor, DNA binding agent, anti-metabolite, cytoskeletal-interacting drug, ionizing radiation, or a combination thereof.
28. The method of any of statements 1-27, wherein the composition further comprises cholesterol, phospholipids, mannose, retinal, a fat soluble vitamin, polyethylene glycol, technetium-99m (^{99m}Tc), hemoglobin, or a combination thereof.
29. The method of any of statements 1-28, wherein the composition is formulated as a liposomal formulation.
30. The method of statement 29, wherein liposomes in the liposomal formulation comprise non-polymer molecules embedded within the liposomal exterior or bound to the exterior of the liposome.
31. The method of any of statements 1-30, wherein the composition is formulated as a liposomal formulation with liposomes that comprise non-polymer molecules embedded within the liposomal exterior or bound to the exterior of the liposome, and wherein the non-polymer molecules are selected from the group consisting of haptens, enzymes, antibodies, antibody fragments, cytokines, hormones, peptides, polypeptides, proteins or a combination thereof.
32. The method of statement 31, wherein the non-polymer molecules bind to a receptor or cell-membrane protein on the surface of a bone marrow cell, a bone marrow-derived cell, a myeloid progenitor cell, or a metastatic tumor cell.
33. The method of any of statements 1-32, further comprising detecting whether the animal has a metastatic tumor.
34. The method of statement 33, wherein detecting comprises testing whether a test sample from the animal expresses at least two-fold higher levels of versican than a negative control sample.
35. The method of statement 34, wherein the test sample is a tissue sample or a bodily fluid.
36. The method of statement 34, wherein the bodily fluid is a serum sample, a plasma sample, a blood sample, a urine sample, a breast milk sample, a lymph sample, or a combination thereof.
37. The method of statement 34, wherein the negative control sample is a non-metastatic sample of the same tissue-type or fluid type as the test sample.
38. A method of inhibiting expression of E-cadherin in animal cells, comprising providing the animal cells with an inhibitor of versican.
39. A method of inhibiting expression of occludin in in animal cells, comprising providing the animal cells with an inhibitor of versican.
40. A method of stimulating expression of vimentin in animal cells, comprising providing the animal cells with an inhibitor of versican.
41. A method of stimulating expression of snail in animal cells, comprising providing the animal cells with an inhibitor of versican.
42. A method of detecting whether an animal has at least one metastatic tumor comprising:
   (a) measuring versican expression levels in a test sample from the animal; and
   (b) detecting that the animal has at least one metastatic tumor when at least two-fold higher levels of versican are expressed in the test sample than in a negative control sample.
43. The method of statement 42, wherein the test sample is a tissue sample or a bodily fluid.
44. The method of statement 42 or 43, wherein the negative control sample is a non-metastatic sample of the same tissue-type or fluid type as the test sample.
45. The method of any of statement 42-44, further comprising administering an anti-cancer agent to the animal when at least two-fold higher levels of versican are expressed in the test sample than in a negative control sample.
46. The method of any of statement 42-45, further comprising administering a versican inhibitor to the animal when at least two-fold higher levels of versican are expressed in the test sample than in a negative control sample.

1. A method of inhibiting establishment or growth of metastatic tumor cells at a site distal from a primary tumor in an animal comprising administering to the animal a composition comprising a versican inhibitor to thereby inhibit establishment or growth of metastatic tumor cells at a site distal from a primary tumor in the animal.

2. The method of claim 1, wherein the versican inhibitor does not affect growth of the primary tumor or the animal has undergone surgery to remove the primary tumor.

3. The method of claim 1, wherein the versican inhibitor is administered to bone marrow or to a site that can have metastatic tumor cells.

4. The method of claim 1, wherein the versican inhibitor inhibits versican expression in bone marrow cells, bone marrow-derived cells or myeloid progenitor cells of the animal.

5. The method of claim 1, wherein the versican inhibitor is formulated to target bone marrow, bone marrow-derived cells or myeloid progenitor cells.

6. The method of claim 1, wherein the method inhibits recruitment of myeloid progenitor cells to a premetastatic or metastatic site in the animal.

7. The method of claim 1, wherein the method inhibits TGF-3/Smad2/3 signaling in the animal.

8. The method of claim 1, wherein the composition further comprises an antibody that specifically binds to CD11b, CD33, VEGF receptor, AFP, CEA, CA-125, MUC-1, ETA, tyrosinase, ras, p53, MAGE1, or combinations of antibodies that specifically bind to any of CD11b, CD33, VEGF receptor, AFP, CEA, CA-125, MUC-1, ETA, tyrosinase, ras, p53, and MAGE1.

9. The method of claim 1, wherein the composition comprises a versican inhibitor selected from the group consisting of budesonide, one or more hyaluronan oligomers, one or more anti-versican antibodies, one or more non-functioning versican peptides, one or more versican inhibitory nucleic acids, and combinations thereof.

10. The method of claim 1, wherein the composition comprises a versican inhibitory nucleic acid that can specifically bind to a versican mRNA under physiological conditions and can inhibit expression or translation of a versican mRNA.

11. The method of claim 1, wherein the composition comprises at least one versican inhibitory nucleic acid with:
(a) a sequence comprising 5′-ACACCAGAATTAG AAAGTTCAA-3′ (shVcn1; SEQ ID NO:43), or 5′-AGCACCTTGTCTGATGGCCAAG-3′ (shVcn2; SEQ ID NO:44);
(b) an RNA sequence corresponding to a DNA sequence comprising 5′-ACACCAGAATTAG AAAGTTCAA-3′ (shVcn1; SEQ ID NO:43), or 5′-AGCACCTTGTCTGATGGCCAAG-3′ (shVcn2; SEQ ID NO:44);
(c) with a DNA or RNA sequence comprising a sequence complementary to 5′-ACACCAGAATTAG AAAGTTCAA-3′ (shVcn1; SEQ ID NO:43), or 5′-AGCACCTTGTCTGATGGCCAAG-3′ (shVcn2; SEQ ID NO:44); or
(d) a combination thereof.

12. The method of claim 1, wherein the composition comprises at least one versican inhibitory peptide with a sequence that has at least 90% sequence identity to a sequence selected from the group consisting of SEQ ID NO:4-41 and 42.

13. The method of claim 1, wherein the composition further comprises an additional therapeutic agent or anti-cancer agent selected from the group consisting of a radioactive drug, topoisomerase inhibitor, DNA binding agent, anti-metabolite, cytoskeletal-interacting drug, ionizing radiation, or a combination thereof.

14. The method of claim 1, wherein the composition further comprises cholesterol, phospholipids, mannose, retinal, a fat soluble vitamin, polyethylene glycol, technetium-99m (^{99m}Tc), hemoglobin, or a combination thereof.

15. The method of claim 1, wherein the composition is formulated as a liposomal formulation.

16. The method of claim 1, wherein the composition is formulated as a liposomal formulation with liposomes that comprise non-polymer molecules embedded within the liposomal exterior or bound to the exterior of the liposome, wherein the non-polymer molecules bind to a receptor or cell-membrane protein on the surface of a bone marrow cell, a bone marrow-derived cell, a myeloid progenitor cell, or a metastatic tumor cell.

17. The method of claim 1, wherein the composition is formulated as a liposomal formulation with liposomes that comprise non-polymer molecules embedded within the liposomal exterior or bound to the exterior of the liposome, wherein the non-polymer molecules are selected from the group consisting of haptens, enzymes, antibodies, antibody fragments, cytokines, hormones, peptides, polypeptides, proteins or a combination thereof.

18. The method of claim 1, further comprising detecting whether the animal has a metastatic tumor.

19. The method of claim 18, wherein detecting comprises testing whether a test sample from the animal expresses at least two-fold higher levels of versican than a negative control sample.

20. The method of claim 19, wherein the test sample is a tissue sample or a bodily fluid.

21. The method of claim 19, wherein the negative control sample is a non-metastatic sample of the same tissue-type or fluid type as the test sample.

22. A method of detecting whether an animal has at least one metastatic tumor comprising:
(a) measuring versican expression levels in a test sample from the animal; and
(b) detecting that the animal has at least one metastatic tumor when at least two-fold higher levels of versican are expressed in the test sample than in a negative control sample.

23. The method of claim 22, wherein the test sample is a tissue sample or a bodily fluid.

24. The method of claim 22, wherein the negative control sample is a non-metastatic sample of the same tissue-type or fluid type as the test sample.

25. The method of claim 22, further comprising administering an anti-cancer agent to the animal when at least two-fold higher levels of versican are expressed in the test sample than in a negative control sample.

26. The method of claim 22, further comprising administering a versican inhibitor to the animal when at least two-fold higher levels of versican are expressed in the test sample than in a negative control sample.
